(19) United States
(12) Patent Application Publication
(10) Pub. No.: US 2024/0301464 A1
(43) Pub. Date: Sep. 12, 2024

(54) CONCURRENT SEQUENCING OF FORWARD AND REVERSE COMPLEMENT STRANDS ON SEPARATE POLYNUCLEOTIDES FOR METHYLATION DETECTION

(71) Applicant: Illumina, Inc., San Diego, CA (US)

(72) Inventors: Aathavan Karunakaran, San Diego, CA (US); Shagesh Sridharan, San Diego, CA (US); Jonathan Mark Boutell, Cambridge (GB); Gery M. Vessere, San Diego, CA (US)

(21) Appl. No.: 18/414,085

(22) Filed: Jan. 16, 2024

Related U.S. Application Data
(60) Provisional application No. 63/439,491, filed on Jan. 17, 2023.

(51) Int. Cl. C12Q 1/6806; C12Q 1/6869

(52) U.S. Cl. CPC C12Q 1/6806; C12Q 1/6869

(57) ABSTRACT

The invention relates to methods of detecting modified cytosines in nucleic acid sequences.

### INCORPORATION BY REFERENCE TO RELATED APPLICATIONS

This application claims the benefit of U.S. Provisional Application No. 63/439,491, filed Jan. 17, 2023, the disclosure of which is hereby expressly incorporated by reference in its entirety and is hereby expressly made a portion of this application.

### REFERENCE TO SEQUENCE LISTING

The present application is being filed along with a Sequence Listing in electronic format. The Sequence Listing is provided as an XML file entitled “ILLINC784A”, created and last saved on Apr. 27, 2024, which is 79,479 bytes in size. The information in the electronic format of the Sequence Listing is hereby incorporated by reference in its entirety.

### FIELD OF THE INVENTION

The invention relates to methods of detecting modified cytosines in nucleic acid sequences.

### BACKGROUND OF THE INVENTION

Modified cytosines, including 5-methylcytosine (5mC), are a well-studied epigenetic modification that play fundamental roles in human development and disease. Its genome-wide distribution differs between tissue types, and between healthy and diseased states. In recent years, 5mC has also gained prominence as a tool for clinical diagnostics: its distribution in cell-free DNA (cfDNA)—obtained from a liquid biopsy—can be used for the tissue-specific prediction of early-stage cancer.

As a result, there has been an intense focus on developing methods for mapping modified cytosines at single base resolution, with minimal loss of sample polynucleotide quantity, quality, and complexity.

However, there remains a need to develop new methods for detecting modified cytosines, and in particular methods that enable quick and accurate detection of modified cytosines.

### SUMMARY OF THE INVENTION

According to an aspect of the present invention, there is provided a method of preparing polynucleotide sequences for detection of modified cytosines, comprising:
synthesising at least one first polynucleotide sequence comprising a first portion and at least one second polynucleotide sequence comprising a second portion,
wherein the at least one first polynucleotide sequence comprising a first portion and the at least one second polynucleotide sequence comprising a second portion each comprise portions of a double-stranded nucleic acid template, and the first portion comprises a forward strand of the template, and the second portion comprises a reverse complement strand of the template; or wherein the first portion comprises a reverse strand of the template, and the second portion comprises a forward complement strand of the template,
wherein the template is generated from a target polynucleotide to be sequenced via complementary base pairing, and wherein the target polynucleotide has been pre-treated using a conversion reagent,
wherein the conversion reagent is configured to convert a modified cytosine to thymine or a nucleobase which is read as thymine/uracil, and/or wherein the conversion reagent is configured to convert an unmodified cytosine to uracil or a nucleobase which is read as thymine/uracil.

In one embodiment, the target polynucleotide has been pre-treated using a conversion reagent configured to convert a modified cytosine to thymine or a nucleobase which is read as thymine/uracil.

In another embodiment, the target polynucleotide has been pre-treated using a conversion reagent configured to convert an unmodified cytosine to uracil or a nucleobase which is read as thymine/uracil.

In a further embodiment, the conversion agent comprises a chemical agent and/or an enzyme.

In one example, the chemical agent comprises a boron-based reducing agent.

In one embodiment, the boron-based reducing agent is an amine-borane compound or an azine-borane compound.

In one embodiment, the boron-based reducing agent is selected from the group consisting of pyridine borane, 2-picoline borane, t-butylamine borane, ammonia borane, ethylenediamine borane and dimethylamine borane.

In one embodiment, the chemical agent comprises sulfite; such as bisulfite or sodium bisulfite.

In one embodiment, the enzyme comprises a cytidine deaminase.

In one embodiment, the cytidine deaminase is a wild-type cytidine deaminase or a mutant cytidine deaminase. In one embodiment, the cytidine deaminase is a mutant cytidine deaminase.

In one embodiment, the cytidine deaminase is a member of the AID subfamily, the APOBEC1 subfamily, the APOBEC2 subfamily, the APOBEC3A subfamily, the APOBEC3B subfamily, the APOBEC3C subfamily, the APOBEC3D subfamily, the APOBEC3F subfamily, the APOBEC3G subfamily, the APOBEC3H subfamily, or the APOBEC4 subfamily. In one embodiment, the cytidine deaminase is a member of the APOBEC3A subfamily.

In one embodiment, the cytidine deaminase comprises amino acid substitution mutations at positions functionally equivalent to (Tyr/Phe) 130 and Tyr132 in a wild-type APOBEC3A protein.

In one example, the (Tyr/Phe) 130 is Tyr130, and the wild-type APOBEC3A protein is SEQ ID NO. 16.

In one embodiment, the substitution mutation at the position functionally equivalent to Tyr130 comprises Ala, Val or Trp.

In one embodiment, the substitution mutation at the position functionally equivalent to Tyr132 comprises a mutation to His, Arg, Gln or Lys.

In one embodiment, the mutant cytidine deaminase comprises a ZDD motif H—[P/A/V]-E-X_{[23-28]}—P—C—X_{[2-4]}—C(SEQ ID NO: 51).

In one embodiment, the mutant cytidine deaminase is a member of the APOBEC3A subfamily and comprises a ZDD motif HXEX₂₄SW(S/T)PCX_{[2-4]}CX₆FX₈LX₅R(L/I)YX_{[8 11]}LX₂LX_{[10]}M (SEQ ID NO: 52).

In one embodiment, the mutant cytidine deaminase converts 5-methylcytosine to thymine by deamination at a greater rate than conversion rate of cytosine to uracil by deamination; wherein the rate may be at least 100-fold greater.

In one embodiment, the target polynucleotide is treated with a further agent prior to treatment with the conversion reagent.

In one embodiment, the further agent is configured to convert a modified cytosine to another modified cytosine.

In one embodiment, the further agent configured to convert a modified cytosine to another modified cytosine comprises a chemical agent and/or an enzyme.

In one embodiment, the further agent configured to convert a modified cytosine to another modified cytosine comprises an oxidising agent; such as a metal-based oxidising agent; for example, a transition metal-based oxidising agent; such as a ruthenium-based oxidising agent.

In one embodiment, the further agent configured to convert a modified cytosine to another modified cytosine comprises a reducing agent; such as a Group III-based reducing agent; for example, a boron-based reducing agent.

In one embodiment, the further agent configured to convert a modified cytosine to another modified cytosine comprises a ten-eleven translocation (TET) methylcytosine dioxygenase; wherein the TET methylcytosine dioxygenase may be a member of the TET1 subfamily, the TET2 subfamily, or the TET3 subfamily.

In one embodiment, the further agent is configured to reduce/prevent deamination of a particular modified cytosine.

In one embodiment, the further agent configured to reduce/prevent deamination of a particular modified cytosine comprises a chemical agent and/or an enzyme.

In one embodiment, the further agent configured to reduce/prevent deamination of a particular modified cytosine comprises a glycosyltransferase; such as a β-glucosyltransferase.

In one embodiment, the further agent configured to reduce/prevent deamination of a particular modified cytosine comprises a hydroxylamine or a hydrazine.

In one embodiment, the modified cytosine is selected from the group consisting of: 5-methylcytosine, 5-hydroxymethylcytosine, 5-formylcytosine and 5-carboxylcytosine.

In one embodiment, the forward strand of the template is not identical to the reverse complement strand of the template.

In one embodiment, the forward strand comprises a guanine base at a first position, and wherein the reverse complement strand comprises an adenine base at a second position corresponding to the same position number as the first position; or wherein the forward strand comprises an adenine base at a first position, and wherein the reverse complement strand comprises a guanine base at a second position corresponding to the same position number as the first position.

In one embodiment, the method further comprises a step of preparing the first portion and the second portion for concurrent sequencing.

In one embodiment, the method comprises simultaneously contacting first sequencing primer binding sites located after a 3′-end of the first portions with first primers and second sequencing primer binding sites located after a 3′-end of the second portions with second primers.

In one embodiment, the method comprises nicking the at least one first polynucleotide sequence and nicking the at least one second polynucleotide sequence.

In one embodiment, a proportion of first portions is capable of generating a first signal and a proportion of second portions is capable of generating a second signal, wherein an intensity of the first signal is substantially the same as an intensity of the second signal.

In one embodiment, the method further comprises a step of selectively processing the at least one first polynucleotide sequence comprising a first portion and the at least one second polynucleotide sequence comprising a second portion, such that a proportion of first portions are capable of generating a first signal and a proportion of second portions are capable of generating a second signal, wherein the selective processing causes an intensity of the first signal to be greater than an intensity of the second signal.

In one embodiment, a concentration of the first portions capable of generating the first signal is greater than a concentration of the second portions capable of generating the second signal.

In one embodiment, a ratio between the concentration of the first portions capable of generating the first signal and the concentration of the second portions capable of generating the second signal is between 1.25:1 to 5:1, or between 1.5:1 to 3:1, or about 2:1.

In one embodiment, selective processing comprises preparing for selective sequencing or conducting selective sequencing.

In one embodiment, selectively processing comprises conducting selective amplification.

In another embodiment, selectively processing comprises contacting first sequencing primer binding sites located after a 3′-end of the first portions with first primers and contacting second sequencing primer binding sites located after a 3′-end of the second portions with second primers, wherein the second primers comprises a mixture of blocked second primers and unblocked second primers.

In one example, the blocked second primer comprises a blocking group at a 3′ end of the blocked second primer.

In one embodiment, the blocking group is selected from the group consisting of: a hairpin loop, a deoxynucleotide, a deoxyribonucleotide, a hydrogen atom instead of a 3′—OH group, a phosphate group, a phosphorothioate group, a propyl spacer, a modification blocking the 3′-hydroxyl group, or an inverted nucleobase.

In one embodiment, the selective processing comprises selectively removing some or substantially all of second immobilised primers that are not yet extended, and conducting a further amplification cycle in order to selectively amplify the first polynucleotide sequence(s) relative to the second polynucleotide sequence(s).

In one embodiment, selectively processing comprises selectively blocking some or substantially all of second immobilised primers that are not yet extended using a primer blocking agent, wherein the primer blocking agent is configured to limit or prevent synthesis of a strand extending from the second immobilised primer, and conducting a further amplification cycle in order to selectively amplify the first polynucleotide sequence(s) relative to the second polynucleotide sequence(s).

In one embodiment, the primer blocking agent is added whilst first polynucleotide sequence(s) are hybridised to the second immobilised primers.

In one embodiment, the method comprises contacting some or substantially all of the second immobilised primers with an extended primer sequence, wherein the extended primer sequence is substantially complementary to the second immobilised primer and further comprises a 5′ additional nucleotide; and adding the primer blocking agent, wherein the primer blocking agent is complementary to the 5′ additional nucleotide.

In one example, the primer blocking agent is a blocked nucleotide.

In one aspect, the blocked nucleotide comprises a blocking group at a 3′ end of the blocked nucleotide.

In one embodiment, the blocking group is selected from the group consisting of: a hairpin loop, a deoxynucleotide, a deoxyribonucleotide, a hydrogen atom instead of a 3′—OH group, a phosphate group, a phosphorothioate group, a propyl spacer, a modification blocking the 3′-hydroxyl group, or an inverted nucleobase.

In one embodiment, the blocked nucleotide is A or G.

In one embodiment, the first signal and the second signal are spatially unresolved.

In one embodiment, the at least one first polynucleotide sequence comprising the first portion and the at least one second polynucleotide sequence comprising the second portion are attached to a solid support, wherein the solid support may be a flow cell.

In one embodiment, the at least one first polynucleotide sequence comprising the first portion and the at least one second polynucleotide sequence comprising the second portion forms a cluster on the solid support.

In one embodiment, the cluster is formed by bridge amplification.

In one example, the at least one first polynucleotide sequence comprising the first portion and the at least one second polynucleotide sequence comprising the second portion form a duoclonal cluster.

In one embodiment, the solid support comprises at least one first immobilised primer and at least one second immobilised primer.

In one embodiment, the first immobilised primer comprises a sequence as defined in SEQ ID NO. 1 or 5, or a variant or fragment thereof; and the second immobilised primer comprises a sequence as defined in SEQ ID NO. 2, or a variant or fragment thereof.

In one embodiment, each first polynucleotide sequence is attached to a first immobilised primer, and wherein each second polynucleotide sequence is attached a second immobilised primer.

In one embodiment, each first polynucleotide sequence comprises a second adaptor sequence and wherein each second polynucleotide sequence comprises a first adaptor sequence, wherein the second adaptor sequence is substantially complementary to the second immobilised primer and wherein the first adaptor sequence is substantially complementary to the first immobilised primer.

In one embodiment, the step of synthesising at least one first polynucleotide sequence comprising a first portion and at least one second polynucleotide sequence comprising a second portion comprises:
synthesising a loop-ligated precursor polynucleotide by connecting a 3′-end of the forward strand of the target polynucleotide and a 5′-end of the reverse strand of the target polynucleotide with a loop, or connecting a 5′-end of the forward strand of the target polynucleotide and a 3′-end of the reverse strand of the target polynucleotide with a loop,
synthesising the at least one first polynucleotide sequence comprising the first portion by forming a complement of the loop-ligated precursor polynucleotide,
synthesising the at least one second polynucleotide sequence comprising the at least one second polynucleotide sequence by forming a complement of the at least one first polynucleotide sequence.

In one embodiment, the method further comprises concurrently sequencing nucleobases in the first portion and the second portion.

According to another aspect of the present invention, there is provided a method of sequencing polynucleotide sequences to detect modified cytosines, comprising:
preparing polynucleotide sequences for detection of modified cytosines using a method as described herein;
concurrently sequencing nucleobases in the first portion and the second portion; and
identifying modified cytosines by detecting differences when comparing a sequence output from the first portion with a sequence output from the second portion.

In one example, the step of concurrently sequencing nucleobases comprises performing sequencing-by-synthesis or sequencing-by-ligation.

In one embodiment, the step of preparing the polynucleotide sequences comprises using a method as described herein; and wherein the step of concurrent sequencing nucleobases in the first portion and the second portion is based on the intensity of the first signal and the intensity of the second signal.

In one embodiment, the method further comprises a step of conducting paired-end reads.

In one embodiment, the step of concurrently sequencing nucleobases comprises:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification of the plurality of classifications represents one or more possible combinations of respective first and second nucleobases, and wherein at least one classification of the plurality of classifications represents more than one possible combination of respective first and second nucleobases; and
(d) based on the selected classification, determining sequence information from the first portion and the second portion.

In one embodiment, selecting the classification based on the first and second intensity data comprises selecting the classification based on the combined intensity of the first and second signal components and the combined intensity of the third and fourth signal components.

In one embodiment, when based on a nucleobase of the same identity, an intensity of the first signal component is substantially the same as an intensity of the second signal component and an intensity of the third signal component is substantially the same as an intensity of the fourth signal component.

In one embodiment, the plurality of classifications consists of a predetermined number of classifications.

In one embodiment, the plurality of classifications comprises:
one or more classifications representing matching first and second nucleobases; and
one or more classifications representing mismatching first and second nucleobases, and
wherein determining sequence information of the first portion and second portion comprises:
   in response to selecting a classification representing matching first and second nucleobases, determining a match between the first and second nucleobases; or
   in response to selecting a classification representing mismatching first and second nucleobases, determining a mismatch between the first and second nucleobases.

In one embodiment, determining sequence information of the first portion and the second portion comprises, in response to selecting a classification representing a match between the first and second nucleobases, base calling the first and second nucleobases.

In one embodiment, determining sequence information of the first portion and the second portion comprises, based on the selected classification, determining that the second portion is modified relative to the first portion at a location associated with the first and second nucleobases.

In one embodiment, the first signal component, second signal component, third signal component and fourth signal component are generated based on light emissions associated with the respective nucleobase.

In one embodiment, the light emissions are detected by a sensor, wherein the sensor is configured to provide a single output based upon the first and second signals.

In one embodiment, the sensor comprises a single sensing element.

In one embodiment, the method further comprises repeating steps (a) to (d) for each of a plurality of base calling cycles.

According to another aspect of the present invention, there is provided a kit comprising instructions for preparing polynucleotide sequences for detection of modified cytosines as described herein, and/or for sequencing polynucleotide sequences to detect modified cytosines as described herein.

According to another aspect of the present invention, there is provided a data processing device comprising means for carrying out a method as described herein.

In one embodiment, the data processing device is a polynucleotide sequencer.

According to another aspect of the present invention, there is provided a computer program product comprising instructions which, when the program is executed by a processor, cause the processor to carry out a method as described herein.

According to another aspect of the present invention, there is provided a computer-readable storage medium comprising instructions which, when executed by a processor, cause the processor to carry out a method as described herein.

According to another aspect of the present invention, there is provided a computer-readable data carrier having stored thereon a computer program product as described herein.

A data carrier signal carrying a computer program product as described herein.

### DESCRIPTION OF THE DRAWINGS

FIG. 1 shows a forward strand, reverse strand, forward complement strand, and reverse complement strand of a polynucleotide molecule.

FIG. 2 shows the steps involved in a loop fork method.

FIG. 3 shows an example of a polynucleotide sequence prepared using a loop fork method.

FIG. 4 shows an example of a polynucleotide sequence prepared using a loop fork method.

FIG. 5 shows a typical solid support.

FIG. 6 shows the stages of bridge amplification for polynucleotide templates prepared using a loop fork method and the generation of an amplified cluster, comprising (A) a concatenated library strand hybridising to a immobilised primer; (B) generation of a template strand from the library strand; (C) dehybridisation and washing away the library strand; (D) generation of a template complement strand from the template strand via bridge amplification and dehybridisation of the sequence bridge; and (E) further amplification to provide a plurality of template and template complement strands.

FIG. 7 shows the detection of nucleobases using 4-channel, 2-channel and 1-channel chemistry.

FIG. 8 shows a method of selective sequencing.

FIG. 9 shows a method of selective amplification comprising (A) starting from a plurality of template and template complement strands; (B) selective cleavage of one type of immobilised primer from the support; (C) only template (or template complement) strands complementary to the free immobilised primer anneal and undergo bridge amplification, (D) producing different proportions of template and template complement strands; (E) subsequent standard (non-selective) sequencing occurs in different proportions enabling signal differentiation.

FIG. 10 shows a method of selective amplification comprising (A) template and template complement strands annealing to immobilised primers; (B) addition of a primer-blocking agent that binds only to one type of immobilised primer, preventing the extension from that one type of immobilised primer, preventing the extension from one type of immobilised primer; (C) producing different proportions of template and template complement strands; (D) subsequent standard (non-selective) sequencing occurs in different proportions enabling signal differentiation.

FIG. 11 shows a method of selective amplification comprising (A) flowing a (or a plurality of) extended primer sequence(s) containing at least one additional 5′ nucleotide across the surface of the solid support; (B) addition of a primer-blocking agent that binds only to one type of immobilised primer and is complementary to the additional 5′ nucleotide of the extended primer sequence, preventing the extension from one type of immobilised primer.

FIG. 12 is a plot showing graphical representations of sixteen distributions of signals generated by polynucleotide sequences according to one embodiment.

FIG. 13 is a flow diagram showing a method for base calling according to one embodiment.

FIG. 14 is a plot showing graphical representations of nine distributions of signals generated by polynucleotide sequences according to one embodiment.

FIG. 15 shows the effect of unmodified cytosine to uracil conversion treatment of a double-stranded polynucleotide, and a scatter plot showing the resulting distributions of signals generated by polynucleotide sequences.

FIG. 16 shows the effect of modified cytosine to thymine conversion treatment of a double-stranded polynucleotide, and a scatter plot showing the resulting distributions of signals generated by polynucleotide sequences.

FIG. 17 shows alternative signal distributions using a different dye-encoding scheme.

FIG. 18 shows alternative signal distributions using a different dye-encoding scheme.

FIG. 19 shows alternative signal distributions using a different dye-encoding scheme.

FIG. 20 is a flow diagram showing a method for determining sequence information according to one embodiment.

FIG. 21 shows a nicking strategy and subsequent sequencing using standard SBS and double stranded SBS (strand displacement SBS).

FIG. 22 shows a nicking strategy and subsequent sequencing using double stranded SBS (strand displacement SBS).

FIG. 23 shows sequencing using sequencing primers.

FIG. 24 shows a method of conducting paired-end reads.

FIG. 25 shows a method of conducting paired-end reads.

FIGS. 26A to 26F show 9 QaM analyses conducted on the signals obtained from Example 1 (library fragments 1 to 6). The x-axis shows signal intensity from a “red” wavelength channel, whilst the y-axis shows signal intensity from a “green” wavelength channel. A CA dye swap has been performed in this MiniSeq run compared to a standard MiniSeq run. G is not associated with any dyes and as such appears contributes no intensity for both “red” and “green” channels. A is associated with a “red” dye and as such contributes intensity to the “red” channel, but not the “green” channel. T is associated with a “green” dye and as such contributes intensity to the “green” channel, but not the “red channel. C is associated with both a “red” dye and a “green” dye, and as such contributes intensity to both the “red” channel and “green” channel. Since the template comprises forward and reverse complement strands that are sequenced simultaneously, the readout will generate (T,T) reads (top left corner), (T,C) reads (top middle), (C,C) reads (top right corner), (G,G) reads (bottom left corner), (G,A) reads (bottom middle), and (A,A) reads (bottom right corner). The top right corner corresponds to a (5-mC)-G base pair, whilst the bottom left corner corresponds to a G-(5-mC) base pair, thus corresponding with the presence of modified cytosines. Groupings are as follows: T in forward strand of library in top left (marked as “T”); C in forward strand of library in top middle (marked as “C”); 5-mC in forward strand of library in top right (marked as “c”); G in forward strand of library and associated with 5-mC in reverse strand of library in bottom left (marked as “g”); G in forward strand of library and associated with C in reverse strand of library in bottom middle (marked as “G”); and A in forward strand of library in bottom right (marked as “A”). In FIGS. 26A to 26C, two scatter-plots are shown: the plot marked “read-color coded” corresponds to assignments for each base to particular groups during the read process; the plot marked “ref-color coded” shows the true assignments for each base to particular groups and is indicative of where errors have occurred in the read process. FIGS. 26D to 26F show combined “read-color coded” and “ref-color coded” plots—where the read and the reference differ, a border is shown for the read assignment, whilst the central portion of the circle shows the actual assignment. In addition, FIGS. 26A to 26F show sequence alignment of the read sequence to the true methylated pUC19 sample-“m” above or below a C represents 5-mC, whilst “m” above or below a G represents G that is base-paired with 5-mC; red boxes indicate errors in read (of sequence or methylation status).

### DETAILED DESCRIPTION

All patents, patent applications, and other publications referred to herein, including all sequences disclosed within these references, are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

The present invention can be used in sequencing, in particular concurrent sequencing. Methodologies applicable to the present invention have been described in WO 08/041002, WO 07/052006, WO 98/44151, WO 00/18957, WO 02/06456, WO 07/107710, WO05/068656, U.S. Ser. No. 13/661,524 and US 2012/0316086, the contents of which are herein incorporated by reference. Further information can be found in U.S. 20060024681, U.S. 20060292611, WO 06/110855, WO 06/135342, WO 03/074734, WO07/010252, WO 07/091077, WO 00/179553, WO 98/44152 and WO 2022/087150, the contents of which are herein incorporated by reference.

As used herein, the term “variant” refers to a variant polypeptide sequence or part of the polypeptide sequence that retains desired function of the full non-variant sequence. For example, a desired function of the immobilised primer retains the ability to bind (i.e. hybridise) to a target sequence.

As used in any aspect described herein, a “variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid sequence. The sequence identity of a variant can be determined using any number of sequence alignment programs known in the art. As an example, Emboss Stretcher from the EMBL-EBI may be used: https://www.ebl.ac.uk/Tools/psa/emboss_stretcher/(using default parameters: pair output format, Matrix=BLOSUM62, Gap open=1, Gap extend=1 for proteins; pair output format, Matrix=DNAfull, Gap open=16, Gap extend=4 for nucleotides).

As used herein, the term “fragment” refers to a functionally active series of consecutive nucleic acids from a longer nucleic acid sequence. The fragment may be at least 99%, at least 95%, at least 90%, at least 80%, at least 70%, at least 60%, at least 50%, at least 40% or at least 30% the length of the longer nucleic acid sequence. A fragment as used herein may also retain the ability to bind (i.e. hybridise) to a target sequence.

Sequencing generally comprises four fundamental steps: 1) library preparation to form a plurality of target polynucleotides for identification; 2) cluster generation to form an array of amplified template polynucleotides; 3) sequencing the cluster array of amplified template polynucleotides; and 4) data analysis to identify characteristics of the target polynucleotides from the amplified template polynucleotide sequences. These steps are described in greater detail below.

### Library Strands and Template Terminology

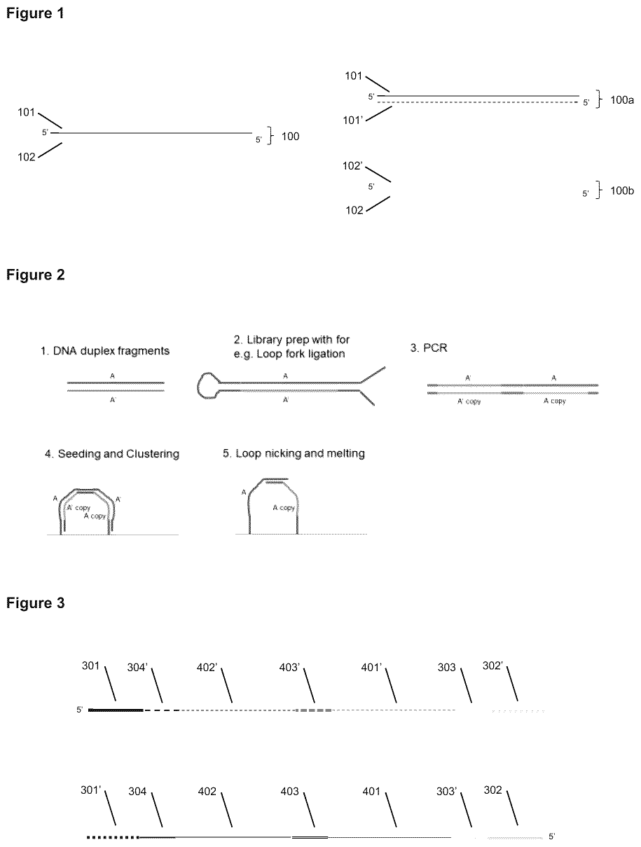

For a given double-stranded polynucleotide sequence 100 to be identified, the polynucleotide sequence 100 comprises a forward strand of the sequence 101 and a reverse strand of the sequence 102. See FIG. 1.

When the polynucleotide sequence 100 is replicated (e.g. using a DNA/RNA polymerase), complementary versions of the forward strand 101 of the sequence 100 and the reverse strand 102 of the sequence 100 are generated. Thus, replication of the polynucleotide sequence 100 provides a double-stranded polynucleotide sequence 100a that comprises a forward strand of the sequence 101 and a forward complement strand of the sequence 101′, and a double-stranded polynucleotide sequence 100b that comprises a reverse strand of the sequence 102 and a reverse complement strand of the sequence 102′.

The term “template” may be used to describe a complementary version of the double-stranded polynucleotide sequence 100. As such, the “template” comprises a forward complement strand of the sequence 101′ and a reverse complement strand of the sequence 102′. Thus, by using the forward complement strand of the sequence 101′ as a template for complementary base pairing, a sequencing process (e.g. a sequencing-by-synthesis or a sequencing-by-ligation process) reproduces information that was present in the original forward strand of the sequence 101. Similarly, by using the reverse complement strand of the sequence 102′ as a template for complementary base pairing, a sequencing process (e.g. a sequencing-by-synthesis or a sequencing-by-ligation process) reproduces information that was present in the original reverse strand of the sequence 102.

The two strands in the template may also be referred to as a forward strand of the template 101′ and a reverse strand of the template 102′. The complement of the forward strand of the template 101′ is termed the forward complement strand of the template 101, whilst the complement of the reverse strand of the template 102′ is termed the reverse complement strand of the template 102.

Generally, where forward strand, reverse strand, forward complement strand, and reverse complement strand are used herein without qualifying whether they are with respect to the original polynucleotide sequence 100 or with respect to the “template”, these terms may be interpreted as referring to the “template”.

| Language for original | |
|---|---|
| polynucleotide sequence | Corresponding language for the |
| 100 | “template” |
| Forward strand of the | Forward complement strand of the |
| sequence 101 | template 101 (sometimes referred to |
| | herein as forward complement strand |
| | 101) |
| Reverse strand of the | Reverse complement strand of the |
| sequence 102 | template 102 (sometimes referred to |
| | herein as reverse complement strand |
| | 102) |
| Forward complement strand | Forward strand of the template 101′ |
| of the sequence 101′ | (sometimes referred to herein as forward |
| | strand 101′) |
| Reverse complement strand | Reverse strand of the template 102′ |
| of the sequence 102′ | (sometimes referred to herein as reverse |
| | strand 102′) |

### Library Preparation

Library preparation is the first step in any high-throughput sequencing platform. These libraries allow templates to be generated via complementary base pairing that can subsequently be clustered and amplified. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adaptors (adaptor sequences). The original sample DNA fragments are referred to as “inserts”. The target polynucleotides may advantageously also be size-fractionated prior to modification with the adaptor sequences.

As described herein, typically the templates to be generated from the libraries may include separate polynucleotide sequences, in particular a first polynucleotide sequence comprising a first portion and a second polynucleotide sequence comprising a second portion. Generating these templates from particular libraries may be performed according to methods known to persons of skill in the art. However, some example approaches of preparing libraries suitable for generation of such templates are described below.

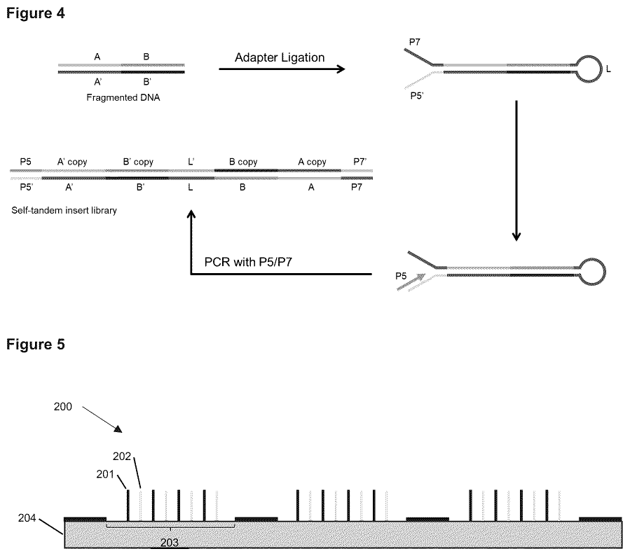

In some embodiments, the library may be prepared using a loop fork method, which is described below. This procedure may be used, for example, for preparing templates including a first polynucleotide sequence comprising a first portion and a second polynucleotide sequence comprising a second portion, wherein the first portion is a forward strand of the template, and the second portion is a reverse complement strand of the template (or alternatively, wherein the first portion is a reverse strand of the template, and the second portion is a forward complement strand of the template). A representative process for conducting a loop fork method is shown in FIG. 2.

Starting from a double-stranded polynucleotide sequence comprising a forward strand of the sequence and a reverse strand of the sequence, adaptors may be ligated to a first end of the sequence (e.g. using processes as described in more detail in e.g. WO 07/052006, or “tagmentation” methods as described above). A second end of the sequence (different from the first end) may be ligated to a loop, which connects the forward strand of the sequence and the reverse strand of the sequence, thus generating a loop fork ligated polynucleotide sequence. Conducting PCR on the loop fork ligated polynucleotide sequence produces a new double-stranded polynucleotide sequence, one strand comprising the forward strand of the sequence and the reverse strand of the sequence, and the other strand comprising a forward complement strand of the sequence and a reverse complement strand of the sequence. The library is now ready for seeding, clustering and amplification.

As will be described later, during clustering and amplification, further processes may be used to generate templates including a first polynucleotide sequence comprising a first portion and a second polynucleotide sequence comprising a second portion, wherein the first portion is a forward strand of the template, and the second portion is a reverse complement strand of the template (or alternatively, wherein the first portion is a reverse strand of the template, and the second portion is a forward complement strand of the template).

The processes described above in relation to loop fork methods generate libraries that have self-tandem insert polynucleotides.

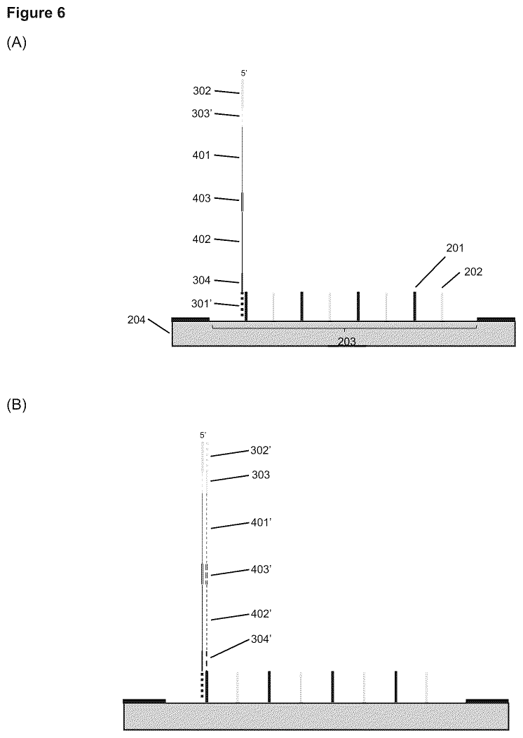

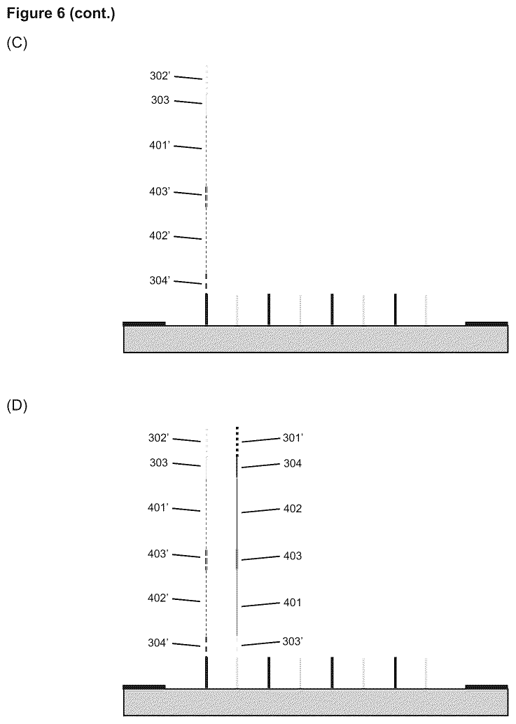

Thus, one strand of a polynucleotide within a polynucleotide library may comprise, in a 5′ to 3′ direction, a second primer-binding complement sequence 302 (e.g. P7), an optional first terminal sequencing primer binding site complement 303′, a first insert sequence 401 (A and B), a loop sequence 403 (L), a second insert sequence 402 (B′ and A′), an optional second terminal sequencing primer binding site 304, and a first primer-binding sequence 301′ (e.g. P5′) (FIGS. 3 and 4—bottom strand).

Alternatively, or in addition, one or more sequencing primer binding sites (or complements) may be provided within the loop complement sequence 403 (L).

Although not shown in FIGS. 3 and 4, the strand may further comprise one or more index sequences. As such, a first index sequence (e.g. i7) may be provided between the second primer-binding complement sequence 302 (e.g. P7) and the optional first terminal sequencing primer binding site complement 303′. Separately, or in addition, a second index complement sequence (e.g. i5′) may be provided between the optional second terminal sequencing primer binding site 304 and the first primer-binding sequence 301′ (e.g. P5′). Thus, in some embodiments, one strand of a polynucleotide within a polynucleotide library may comprise, in a 5′ to 3′ direction, a second primer-binding complement sequence 302 (e.g. P7), a first index sequence (e.g. i7), an optional first terminal sequencing primer binding site complement 303′, a first insert sequence 401 (A and B), a loop sequence 403 (L), a second insert sequence 402 (B′ and A′), an optional second terminal sequencing primer binding site 304, a second index complement sequence (e.g. i5′), and a first primer-binding sequence 301′ (e.g. P5′).

Alternatively, or in addition, one or more index sequences (or complements) may be provided within the loop sequence 403 (L).

Another strand of a polynucleotide within a polynucleotide library may comprise, in a 5′ to 3′ direction, a first primer-binding complement sequence 301 (e.g. P5), an optional second terminal sequencing primer binding site complement 304′, a second insert complement sequence 402′ (A′ copy and B′ copy), a loop complement sequence 403′ (L′), a first insert complement sequence 401′ (B copy and A copy), an optional first terminal sequencing primer binding site 303, and a second primer-binding sequence 302′ (e.g. P7′) (FIGS. 3 and 4—top strand).

Alternatively, or in addition, one or more sequencing primer binding sites (or complements) may be provided within the loop complement sequence 403′ (L′).

Although not shown in FIGS. 3 and 4, the another strand may further comprise one or more index sequences. As such, a second index sequence (e.g. i5) may be provided between the first primer-binding complement sequence 301 (e.g. P5) and the optional second terminal sequencing primer binding site complement 304′. Separately, or in addition, a first index complement sequence (e.g. i7′) may be provided between the optional first terminal sequencing primer binding site 303 and the second primer-binding sequence 302′ (e.g. P7′). Thus, in some embodiments, another strand of a polynucleotide within a polynucleotide library may comprise, in a 5′ to 3′ direction, a first primer-binding complement sequence 301 (e.g. P5), a second index sequence (e.g. i5), an optional second terminal sequencing primer binding site complement 304′, a second insert complement sequence 402′ (A′ copy and B′ copy), a loop complement sequence 403′ (L′), a first insert complement sequence 401′ (B copy and A copy), an optional first terminal sequencing primer binding site 303, a first index complement sequence (e.g. i7′), and a second primer-binding sequence 302′ (e.g. P7′).

Alternatively, or in addition, one or more index sequences (or complements) may be provided within the loop complement sequence 403′ (L′).

In one embodiment, the first insert sequence 401 may comprise a forward strand of the sequence 101, and the second insert complement sequence 402′ may comprise a reverse complement strand of the sequence 102′ (or the first insert sequence 401 may comprise a reverse strand of the sequence 102, and the second insert complement sequence 402′ may comprise a forward complement strand of the sequence 101′), for example where the library is prepared using a loop fork method.

Although FIG. 3 shows the presence of a first terminal sequencing primer binding site complement 303′, a second terminal sequencing primer binding site 304, a second terminal sequencing primer binding site complement 304′, and a first terminal sequencing primer binding site 303, these are optional as mentioned above. Accordingly, these sections may be omitted from the library.

As will be understood by the skilled person, a double-stranded nucleic acid will typically be formed from two complementary polynucleotide strands comprised of deoxyribonucleotides or ribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

A sequence comprising at least a primer-binding sequence (a primer-binding sequence and a sequencing primer binding site, or a combination of a primer-binding sequence, an index sequence and a sequencing primer binding site) may be referred to herein as an adaptor sequence, and an insert is flanked by a 5′ adaptor sequence and a 3′ adaptor sequence. The primer-binding sequence may also comprise a sequencing primer for the index read.

As used herein, an “adaptor” refers to a sequence that comprises a short sequence-specific oligonucleotide that is ligated to the 5′ and 3′ ends of each DNA (or RNA) fragment in a sequencing library as part of library preparation. The adaptor sequence may further comprise non-peptide linkers.

In a further embodiment, the P5′ and P7′ primer-binding sequences are complementary to short primer sequences (or lawn primers) present on the surface of a flow cell. Binding of P5′ and P7′ to their complements (P5 and P7) on—for example—the surface of the flow cell, permits nucleic acid amplification. As used herein denotes the complementary strand.

The primer-binding sequences in the adaptor which permit hybridisation to amplification primers (e.g. lawn primers) will typically be around 20-40 nucleotides in length, although the invention is not limited to sequences of this length. The precise identity of the amplification primers (e.g. lawn primers), and hence the cognate sequences in the adaptors, are generally not material to the invention, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA (or RNA) sequences that are added to each DNA (or RNA) fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in WO05/068656, whose contents are incorporated herein by reference in their entirety. The tag can be read at the end of the first read, or equally at the end of the second read, for example using a sequencing primer complementary to the strand marked P7. The invention is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example WO 2008/093098, which is incorporated herein by reference. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing primer binding sites are sequencing and/or index primer binding sites and indicate the starting point of the sequencing read. During the sequencing process, a sequencing primer anneals (i.e. hybridises) to at least a portion of the sequencing primer binding site on the template strand. The polymerase enzyme binds to this site and incorporates complementary nucleotides base by base into the growing opposite strand.

### Cluster Generation and Amplification

Once a double stranded nucleic acid library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 4th Ed, Cold Spring Harbor Laboratory Press, Cold Spring Harbor Laboratory Press, NY; Current Protocols, eds Ausubel et al). In one embodiment, chemical denaturation may be used.

Following denaturation, a single-stranded library may be contacted in free solution onto a solid support comprising surface capture moieties (for example P5 and P7 lawn primers).

Thus, embodiments of the present invention may be performed on a solid support 200, such as a flowcell. However, in alternative embodiments, seeding and clustering can be conducted off-flowcell using other types of solid support.

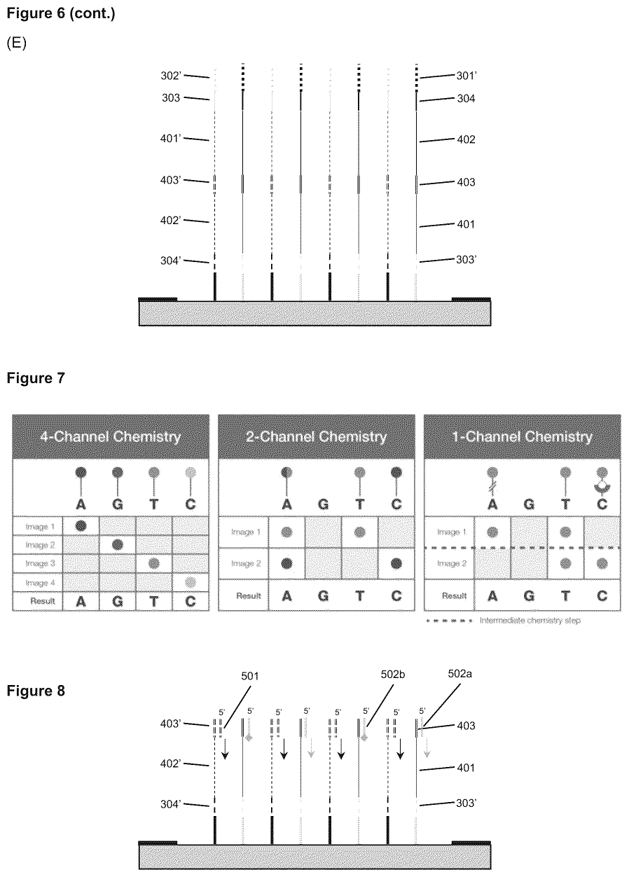

The solid support 200 may comprise a substrate 204. See FIG. 5. The substrate 204 comprises at least one well 203 (e.g. a nanowell), and typically comprises a plurality of wells 203 (e.g. a plurality of nanowells).

In one embodiment, the solid support comprises at least one first immobilised primer and at least one second immobilised primer.

Thus, each well 203 may comprise at least one first immobilised primer 201, and typically may comprise a plurality of first immobilised primers 201. In addition, each well 203 may comprise at least one second immobilised primer 202, and typically may comprise a plurality of second immobilised primers 202. Thus, each well 203 may comprise at least one first immobilised primer 201 and at least one second immobilised primer 202, and typically may comprise a plurality of first immobilised primers 201 and a plurality of second immobilised primers 202.

The first immobilised primer 201 may be attached via a 5′-end of its polynucleotide chain to the solid support 200. When extension occurs from first immobilised primer 201, the extension may be in a direction away from the solid support 200.

The second immobilised primer 202 may be attached via a 5′-end of its polynucleotide chain to the solid support 200. When extension occurs from second immobilised primer 202, the extension may be in a direction away from the solid support 200.

The first immobilised primer 201 may be different to the second immobilised primer 202 and/or a complement of the second immobilised primer 202. The second immobilised primer 202 may be different to the first immobilised primer 201 and/or a complement of the first immobilised primer 201.

The (or each of the) first immobilised primer(s) 201 may comprise a sequence as defined in SEQ ID NO. 1 or 5, or a variant or fragment thereof. The second immobilised primer(s) 202 may comprise a sequence as defined in SEQ ID NO. 2, or a variant or fragment thereof.

By way of brief example, following attachment of the P5 and P7 primers to the solid support, the solid support may be contacted with the template to be amplified under conditions which permit hybridisation (or annealing-such terms may be used interchangeably) between the template and the immobilised primers. The template is usually added in free solution under suitable hybridisation conditions, which will be apparent to the skilled reader. Typically, hybridisation conditions are, for example, 5×SSC at 40° C. However, other temperatures may be used during hybridisation, for example about 50° C. to about 75° C., about 55° C. to about 70° C., or about 60° C. to about 65° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either P5′ or P7′) which is capable of bridging to the second primer molecule immobilised on the solid support and binding. Further rounds of amplification (analogous to a standard PCR reaction) leads to the formation of clusters or colonies of template molecules bound to the solid support. This is called clustering.

Thus, solid-phase amplification by either a method analogous to that of WO 98/44151 or that of WO 00/18957 (the contents of which are incorporated herein in their entirety by reference) will result in production of a clustered array comprised of colonies of “bridged” amplification products. This process is known as bridge amplification. Both strands of the amplification products will be immobilised on the solid support at or near the 5′ end, this attachment being derived from the original attachment of the amplification primers. Typically, the amplification products within each colony will be derived from amplification of a single template molecule. Other amplification procedures may be used, and will be known to the skilled person. For example, amplification may be isothermal amplification using a strand displacement polymerase; or may be exclusion amplification as described in WO 2013/188582. Further information on amplification can be found in WO 02/06456 and WO 07/107710, the contents of which are incorporated herein in their entirety by reference.

Through such approaches, a cluster of template molecules is formed, comprising copies of a template strand and copies of the complement of the template strand.

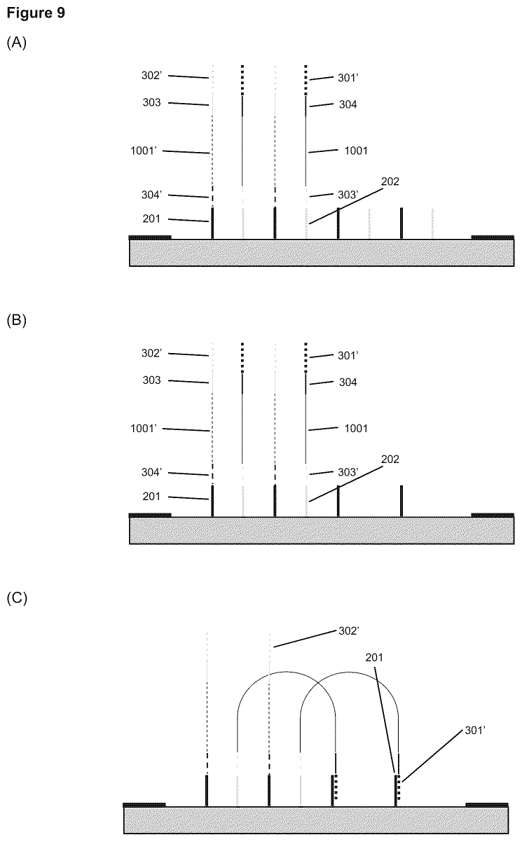

The steps of cluster generation and amplification for templates including a first polynucleotide sequence comprising a first portion and a second polynucleotide sequence comprising a second portion are illustrated below and in FIG. 6.

In cases where (separate) polynucleotide strands are used, each first polynucleotide sequence may be attached (via the 5′-end of the first polynucleotide sequence) to a first immobilised primer, and wherein each second polynucleotide sequence is attached (via the 5′-end of the second polynucleotide sequence) to a second immobilised primer. Each first polynucleotide sequence may comprise a second adaptor sequence, wherein the second adaptor sequence comprises a portion which is substantially complementary to the second immobilised primer (or is substantially complementary to the second immobilised primer). The second adaptor sequence may be at a 3′-end of the first polynucleotide sequence. Each second polynucleotide sequence may comprise a first adaptor sequence, wherein the first adaptor sequence comprises a portion which is substantially complementary to the first immobilised primer (or is substantially complementary to the first immobilised primer). The first adaptor sequence may be at a 3′-end of the second polynucleotide sequence.

In an embodiment, a solution comprising a polynucleotide library prepared by a loop fork method as described above may be flowed across a flowcell.

A particular polynucleotide strand from the polynucleotide library to be sequenced comprising, in a 5′ to 3′ direction, a second primer-binding complement sequence 302 (e.g. P7), an optional first terminal sequencing primer binding site complement 303′, a first insert sequence 401 (A′ copy and B′ copy), a loop complement sequence 403 (L′), a second insert sequence 402 (B copy and A copy), an optional second terminal sequencing primer binding site 304, and a first primer-binding sequence 301′ (e.g. P5′), may anneal (via the first primer-binding sequence 301′) to the first immobilised primer 201 (e.g. P5 lawn primer) located within a particular well 203 (FIG. 6A).

The polynucleotide library may comprise other polynucleotide strands with different first insert sequences 401 and second insert sequences 402. Such other polynucleotide strands may anneal to corresponding first immobilised primers 201 (e.g. P5 lawn primers) in different wells 203, thus enabling parallel processing of the various different strands within the polynucleotide library.

A new polynucleotide strand may then be synthesised, extending from the first immobilised primer 201 (e.g. P5 lawn primer) in a direction away from the substrate 204. By using complementary base-pairing, this generates a template strand comprising, in a 5′ to 3′ direction, the first immobilised primer 201 (e.g. P5 lawn primer) which is attached to the solid support 200, an optional second terminal sequencing primer binding site complement 304′, a second insert complement sequence 402′ (A′ copy and B′ copy), a loop complement sequence 403′ (L′), a first insert complement sequence 401′ (B copy and A copy), an optional first terminal sequencing primer binding site 303, and a second primer-binding sequence 302′ (e.g. P7′) (FIG. 6B). Such a process may utilise a polymerase, such as a DNA or RNA polymerase.

If the polynucleotides in the library comprise index sequences, then corresponding index sequences are also produced in the template.

The polynucleotide strand from the polynucleotide library may then be dehybridised and washed away, leaving a template strand attached to the first immobilised primer 201 (e.g. P5 lawn primer) (FIG. 6C).

The second primer-binding sequence 302′ (e.g. P7′) on the template strand may then anneal to a second immobilised primer 202 (e.g. P7 lawn primer) located within the well 203. This forms a “bridge”.

A new polynucleotide strand may then be synthesised by bridge amplification, extending from the second immobilised primer 202 (e.g. P7 lawn primer) (initially) in a direction away from the substrate 204. By using complementary base-pairing, this generates a template strand comprising, in a 5′ to 3′ direction, the second immobilised primer 202 (e.g. P7 lawn primer) which is attached to the solid support 200, an optional first terminal sequencing primer binding site complement 303′, a first insert sequence 401 (A and B), a loop sequence 403 (L), a second insert sequence 402 (B′ and A′), an optional second terminal sequencing primer binding site 304, and a first primer-binding sequence 301′ (e.g. P5′). Again, such a process may utilise a polymerase, such as a DNA or RNA polymerase.

The strand attached to the second immobilised primer 202 (e.g. P7 lawn primer) may then be dehybridised from the strand attached to the first immobilised primer 201 (e.g. P5 lawn primer) (FIG. 6D).

A subsequent bridge amplification cycle can then lead to amplification of the strand attached to the first immobilised primer 201 (e.g. P5 lawn primer) and the strand attached to the second immobilised primer 202 (e.g. P7 lawn primer). The second primer-binding sequence 302′ (e.g. P7′) on the template strand attached to the first immobilised primer 201 (e.g. P5 lawn primer) may then anneal to another second immobilised primer 202 (e.g. P7 lawn primer) located within the well 203. In a similar fashion, the first primer-binding sequence 301′ (e.g. P5′) on the template strand attached to the second immobilised primer 202 (e.g. P7 lawn primer) may then anneal to another first immobilised primer 201 (e.g. P5 lawn primer) located within the well 203.

Completion of bridge amplification and dehybridisation may then provide an amplified cluster, thus providing a plurality of polynucleotide sequences comprising a first insert complement sequence 401′ and a second insert complement sequence 402′, as well as a plurality of polynucleotide sequences comprising a first insert sequence 401 and a second insert sequence 402 (FIG. 6E).

If desired, further bridge amplification cycles may be conducted to increase the number of polynucleotide sequences within the well 203.

Once again, although FIG. 6 shows the presence of a first terminal sequencing primer binding site complement 303′, a second terminal sequencing primer binding site 304, a second terminal sequencing primer binding site complement 304′, and a first terminal sequencing primer binding site 303, these are optional as mentioned above. Accordingly, these sections may be omitted from the template and template complement strands.

The methods for clustering and amplification described above generally relate to conducting non-selective amplification. However, methods of the present invention relating to selective processing may comprise conducting selective amplification, which is described in further detail below under selective processing.

### Sequencing

As described herein, the template provides information (e.g. identification of the genetic sequence, identification of epigenetic modifications) on the original target polynucleotide sequence. For example, a sequencing process (e.g. a sequencing-by-synthesis or sequencing-by-ligation process) may reproduce information that was present in the original target polynucleotide sequence, by using complementary base pairing.

In one embodiment, sequencing may be carried out using any suitable “sequencing-by-synthesis” technique, wherein nucleotides are added successively in cycles to the free 3′ hydroxyl group, resulting in synthesis of a polynucleotide chain in the 5′ to 3′ direction. The nature of the nucleotide added may be determined after each addition. One particular sequencing method relies on the use of modified nucleotides that can act as reversible chain terminators. Such reversible chain terminators comprise removable 3′ blocking groups. Once such a modified nucleotide has been incorporated into the growing polynucleotide chain complementary to the region of the template being sequenced there is no free 3′—OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB2007/001770, the contents of which are incorporated herein by reference in their entirety. Alternatively, a separate reaction may be carried out containing each of the modified nucleotides added individually.

The modified nucleotides may carry a label to facilitate their detection. Such a label may be configured to emit a signal, such as an electromagnetic signal, or a (visible) light signal.

In a particular embodiment, the label is a fluorescent label (e.g. a dye). Thus, such a label may be configured to emit an electromagnetic signal, or a (visible) light signal. One method for detecting the fluorescently labelled nucleotides comprises using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the contents of which are incorporated herein by reference in their entirety.

However, the detectable label need not be a fluorescent label. Any label can be used which allows the detection of the incorporation of the nucleotide into the DNA sequence.

Each cycle may involve simultaneous delivery of four different nucleotide types to the array of template molecules. Alternatively, different nucleotide types can be added sequentially and an image of the array of template molecules can be obtained between each addition step.

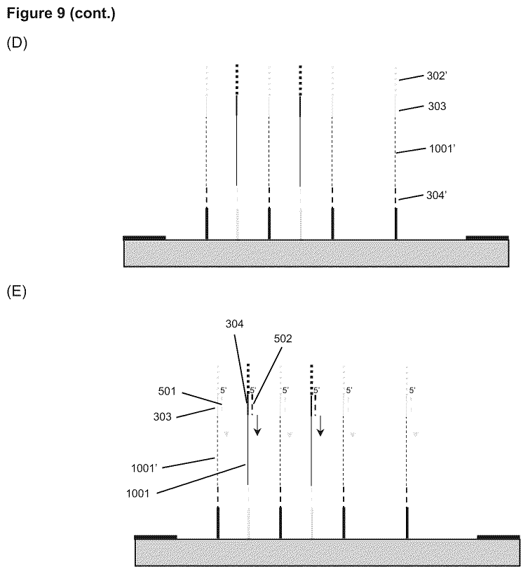

In some embodiments, each nucleotide type may have a (spectrally) distinct label. In other words, four channels may be used to detect four nucleobases (also known as 4-channel chemistry) (FIG. 7—left). For example, a first nucleotide type (e.g. A) may include a first label (e.g. configured to emit a first wavelength, such as red light), a second nucleotide type (e.g. G) may include a second label (e.g. configured to emit a second wavelength, such as blue light), a third nucleotide type (e.g. T) may include a third label (e.g. configured to emit a third wavelength, such as green light), and a fourth nucleotide type (e.g. C) may include a fourth label (e.g. configured to emit a fourth wavelength, such as yellow light). Four images can then be obtained, each using a detection channel that is selective for one of the four different labels. For example, the first nucleotide type (e.g. A) may be detected in a first channel (e.g. configured to detect the first wavelength, such as red light), the second nucleotide type (e.g. G) may be detected in a second channel (e.g. configured to detect the second wavelength, such as blue light), the third nucleotide type (e.g. T) may be detected in a third channel (e.g. configured to detect the third wavelength, such as green light), and the fourth nucleotide type (e.g. C) may be detected in a fourth channel (e.g. configured to detect the fourth wavelength, such as yellow light). Although specific pairings of bases to signal types (e.g. wavelengths) are described above, different signal types (e.g. wavelengths) and/or permutations may also be used.

In some embodiments, detection of each nucleotide type may be conducted using fewer than four different labels. For example, sequencing-by-synthesis may be performed using methods and systems described in US 2013/0079232, which is incorporated herein by reference.

Thus, in some embodiments, two channels may be used to detect four nucleobases (also known as 2-channel chemistry) (FIG. 7—middle). For example, a first nucleotide type (e.g. A) may include a first label (e.g. configured to emit a first wavelength, such as green light) and a second label (e.g. configured to emit a second wavelength, such as red light), a second nucleotide type (e.g. G) may not include the first label and may not include the second label, a third nucleotide type (e.g. T) may include the first label (e.g. configured to emit the first wavelength, such as green light) and may not include the second label, and a fourth nucleotide type (e.g. C) may not include the first label and may include the second label (e.g. configured to emit the second wavelength, such as red light). Two images can then be obtained, using detection channels for the first label and the second label. For example, the first nucleotide type (e.g. A) may be detected in both a first channel (e.g. configured to detect the first wavelength, such as red light) and a second channel (e.g. configured to detect the second wavelength, such as green light), the second nucleotide type (e.g. G) may not be detected in the first channel and may not be detected in the second channel, the third nucleotide type (e.g. T) may be detected in the first channel (e.g. configured to detect the first wavelength, such as red light) and may not be detected in the second channel, and the fourth nucleotide type (e.g. C) may not be detected in the first channel and may be detected in the second channel (e.g. configured to detect the second wavelength, such as green light). Although specific pairings of bases to signal types (e.g. wavelengths) and/or combinations of channels are described above, different signal types (e.g. wavelengths) and/or permutations may also be used.

In some embodiments, one channel may be used to detect four nucleobases (also known as 1-channel chemistry) (FIG. 7—right). For example, a first nucleotide type (e.g. A) may include a cleavable label (e.g. configured to emit a wavelength, such as green light), a second nucleotide type (e.g. G) may not include a label, a third nucleotide type (e.g. T) may include a non-cleavable label (e.g. configured to emit the wavelength, such as green light), and a fourth nucleotide type (e.g. C) may include a label-accepting site which does not include the label. A first image can then be obtained, and a subsequent treatment carried out to cleave the label attached to the first nucleotide type, and to attach the label to the label-accepting site on the fourth nucleotide type. A second image may then be obtained. For example, the first nucleotide type (e.g. A) may be detected in a channel (e.g. configured to detect the wavelength, such as green light) in the first image and not detected in the channel in the second image, the second nucleotide type (e.g. G) may not be detected in the channel in the first image and may not be detected in the channel in the second image, the third nucleotide type (e.g. T) may be detected in the channel (e.g. configured to detect the wavelength, such as green light) in the first image and may be detected in the channel (e.g. configured to detect the wavelength, such as green light) in the second image, and the fourth nucleotide type (e.g. C) may not be detected in the channel in the first image and may be detected in the channel in the second image (e.g. configured to detect the wavelength, such as green light). Although specific pairings of bases to signal types (e.g. wavelengths) and/or combinations of images are described above, different signal types (e.g. wavelengths), images and/or permutations may also be used.

In one embodiment, the sequencing process comprises a first sequencing read and second sequencing read. The first sequencing read and the second sequencing read may be conducted concurrently. In other words, the first sequencing read and the second sequencing read may be conducted at the same time.

The first sequencing read may comprise the binding of a first sequencing primer (also known as a read 1.1 sequencing primer) to the first sequencing primer binding site (e.g. within loop complement sequence 403′). The second sequencing read may comprise the binding of a second sequencing primer (also known as a read 1.2 sequencing primer) to the second sequencing primer binding site (e.g. within loop sequence 403).

This leads to sequencing of the first portion (e.g. second insert complement sequence 402′) and the second portion (e.g. first insert sequence 401).

Other embodiments may involve strand displacement sequencing-by-synthesis (strand displacement SBS). In such a case, a strand displacement polymerase may initiate SBS from a nick. Further examples of strand displacement SBS are described in greater detail below.

Alternative methods of sequencing include sequencing by ligation, for example as described in U.S. Pat. No. 6,306,597 or WO 06/084132, the contents of which are incorporated herein by reference.

The methods for sequencing described above generally relate to conducting non-selective sequencing. However, methods of the present invention relating to selective processing may comprise conducting selective sequencing, which is described in further detail below under selective processing.

### Selective Processing Methods

In some embodiments, selective processing methods may be used to generate signals of different intensities. Accordingly, in some embodiments, the method may comprise selectively processing the at least one first polynucleotide sequence comprising a first portion and the at least one second polynucleotide sequence comprising a second portion, such that a proportion of first portions are capable of generating a first signal and a proportion of second portions are capable of generating a second signal, wherein the selective processing causes an intensity of the first signal to be greater than an intensity of the second signal.

The method may comprise selectively processing a plurality of first polynucleotide sequences each comprising a first portion and a plurality of second polynucleotide sequences each comprising a second portion, such that a proportion of first portions are capable of generating a first signal and a proportion of second portions are capable of generating a second signal, wherein the selective processing causes an intensity of the first signal to be greater than an intensity of the second signal.

By “selective processing” is meant here performing an action that changes relative properties of the first portion and the second portion in the at least one first polynucleotide sequence comprising a first portion and at least one second polynucleotide sequence comprising a second portion (or the plurality of first polynucleotide sequences each comprising a first portion and the plurality of second polynucleotide sequences each comprising a second portion), so that the intensity of the first signal is greater than the intensity of the second signal. The property may be, for example, a concentration of first portions capable of generating the first signal relative to a concentration of second portions capable of generating the second signal. The action may include, for example, conducting selective amplification, conducting selective sequencing, or preparing for selective sequencing.

In one embodiment, the selective processing results in the concentration of the first portions capable of generating the first signal being greater than the concentration of the second portions capable of generating the second signal. In other words, the method of the invention results in an altered ratio of R1:R2 molecules, such as within a single cluster or a single well.

In one embodiment, the ratio may be between 1.25:1 to 5:1, or between 1.5:1 to 3:1, or about 2:1.

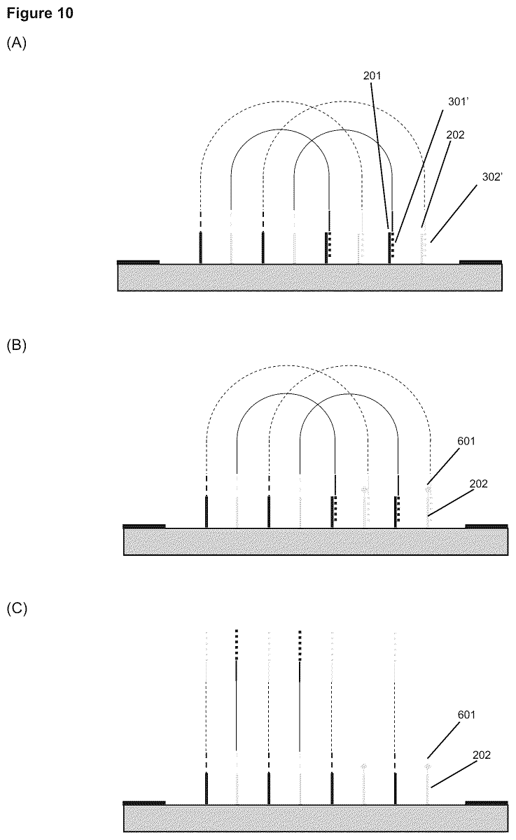

Selective processing may refer to conducting selective sequencing. Alternatively, selective processing may refer to preparing for selective sequencing. As shown in FIG. 8, in one example, selective sequencing may be achieved using a mixture of unblocked and blocked sequencing primers.

Where the method of the invention involves (separate) polynucleotide strands, with a first polynucleotide strand with a first portion, and a second polynucleotide strand with a second portion, the first polynucleotide strand may comprise a first sequencing primer binding site, and the second polynucleotide strand may comprise a second sequencing primer binding site, where the first sequencing primer binding site and second sequencing primer binding site are of a different sequence to each other and bind different sequencing primers.

In one embodiment, binding of first sequencing primers to the first sequencing primer site generates a first signal and binding of second sequencing primers to the second sequencing primer site generates a second signal, where the intensity of the first signal is greater than the intensity of the second signal. This may be applied to embodiments where the first polynucleotide strand comprises a first sequencing primer binding site, and the second polynucleotide strand comprises a second sequencing primer binding site. This is achieved using a mixed population of blocked and unblocked second sequencing primers that bind the second sequencing primer site. Any ratio of blocked:unblocked second primers can be used that generates a second signal that is of a lower intensity than the first signal, for example, the ratio of blocked:unblocked primers may be: 20:80 to 80:20, or 1:2 to 2:1.

In one embodiment, a ratio of 50:50 of blocked:unblocked second primers is used, which in turn generates a second signal that is around 50% of the intensity of the first signal.

The first and second sequencing primers may be added to the flow cell at the same time, or separately but sequentially.

By “blocked” is meant that the sequencing primer comprises a blocking group at a 3′ end of the sequencing primer. Suitable blocking groups include a hairpin loop (e.g. a polynucleotide attached to the 3′-end, comprising in a 5′ to 3′ direction, a cleavable site such as a nucleotide comprising uracil, a loop portion, and a complement portion, wherein the complement portion is substantially complementary to all or a portion of the immobilised primer), a deoxynucleotide, a deoxyribonucleotide, a hydrogen atom instead of a 3′—OH group, a phosphate group, a phosphorothioate group, a propyl spacer (e.g. —O—(CH₂)₃—OH instead of a 3′—OH group), a modification blocking the 3′-hydroxyl group (e.g. hydroxyl protecting groups, such as silyl ether groups (e.g. trimethylsilyl, triethylsilyl, triisopropylsilyl, t-butyl(dimethyl)silyl, t-butyl(diphenyl)silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl)), or an inverted nucleobase. However, the blocking group may be any modification that prevents extension (i.e. elongation) of the primer by a polymerase.

The sequence of the sequencing primers and the sequence primer binding sites are not material to the methods of the invention, as long as the sequencing primers are able to bind to the sequence primer binding site to enable amplification and sequencing of the regions to be identified.

In one aspect, the unblocked and blocked second sequencing primers are present in the sequencing composition in equal concentrations. That is, the ratio of blocked:unblocked second sequencing primers is around 50:50. The sequencing composition may further comprise at least one additional (first) sequencing primer. In one example, the sequencing composition comprises blocked second sequencing primers, unblocked second sequencing primers and at least one first sequencing primer.

As shown in FIG. 8, selective sequencing may be conducted on the amplified (duoclonal) cluster shown in FIG. 6E, after restriction sites in the loop complement sequence 403′ and the loop sequence 403 are cleaved by an endonuclease, as described in further detail below. A plurality of first sequencing primers 501 are added. These sequencing primers 501 anneal to a sequencing primer binding site present in the loop complement sequence 403′. A plurality of second unblocked sequencing primers 502a and a plurality of second blocked sequencing primers 502b are added, either at the same time as the first sequencing primers 501, or sequentially (e.g. prior to or after addition of first sequencing primers 501). These second unblocked sequencing primers 502a and second blocked sequencing primers 502b anneal to a sequencing primer binding site present in the loop sequence 403. This then allows the second insert complement sequences 402′ (i.e. “first portions”) to be sequenced and the first insert sequences 401 (i.e. “second portions”) to be sequenced, wherein a greater proportion of second insert complement sequences 402′ are sequenced (black arrow) compared to a proportion of first insert sequences 401 (grey arrow).

In other embodiments, the positioning of first sequencing primers and second sequencing primers may be swapped. In other words, the first sequencing binding primers may anneal instead to the loop sequence 403, and the second sequencing binding primers may anneal instead to the loop complement sequence 403′.

Alternatively, or in addition, selective processing may refer to selective amplification. That is, selectively amplifying one portion (e.g. the first or second portion) on a first or second polynucleotide strand.

In one example, selective processing comprises selectively removing some or substantially all of second immobilised primers that have not yet been extended (extended to form a second polynucleotide strand), and conducting at least one further amplification cycle in order to selectively amplify the first polynucleotide sequence(s) relative to the second polynucleotide sequence(s). Immobilised primers that have not yet been extended may be referred to herein as free or un-extended second immobilised primers.

Accordingly, in this example, selective removal of some or substantially all free second immobilised primers is carried out before at least one further round of bridge amplification and before any sequencing of the target regions. As a consequence, the ratio of first polynucleotide capable of generating a first signal to the second polynucleotide that is capable of generating a second signal is altered, which in turn leads to two signals of different intensities, permitting concurrent sequencing of both sequences (or the target regions within those sequences).

By “some or substantially all” is meant that at least 75%, at least 80%, at least 90% or between 95% and 100% of free second immobilised primers are removed.

The selective removal of all or substantially all free second immobilised primers may be carried out using a reagent capable of cleaving the immobilised primer from the solid support. This reagent may be added following at least 5, at least 10, at least 15 or following at least 20 to 24 rounds of bridge amplification. The reagent may be added separately or together with the amplification reagents for performing the at least one further round of amplification.

As described above, and described in further detail in WO 2008/041002, the first and second immobilised primers may be attached to the surface of a solid support though a linker. The linker may be different for the first and second immobilised primers. The linker may be any cleavable linker; that is the linker may comprise one or more moieties, such as modified nucleotides, that enable selective cleavage of the immobilised primer from the surface of the solid support. By way of non-limiting example, the linker may comprise uracil bases, phosphorothioate groups, ribonucleotides, diol linkages, disulphide linkages, peptides etc. which may be included, not only to allow covalent attachment to a solid support, but also to allow selective cleavage of the linker.

In one example, the first immobilised primer is attached to a solid support though a first linker, where the linker comprises 8-oxoguanine. In this example, free first immobilised primers (that is, primers that are not extended) can be removed using a FPG glycosylase.

In one example, the sequence of the first immobilised primer comprises the following sequence or a variant of fragment thereof:

(SEQ ID NO: 53)
5′-PS-TTTTTTTTTTAATGATACGGCGACCACCGAUCTACAC-3′
where U = 2-deoxyuridine

In another example, the second immobilised primer is attached to a solid support through a second linker, where the linker comprises uracil or 2-deoxyuridine. In this example, free second immobilised primers (that is, primers that are not extended) can be removed using uracil glycosylase. In one embodiment, free second immobilised primers can be removed using a USER enzyme mix (which is a cocktail of uracil glycosylase and endonuclease VIII).

In one example, the sequence of the second immobilised primer comprises the following sequence or a variant of fragment thereof:

(SEQ ID NO: 54)
5′-PS-TTTTTTTTTTCAAGCAGAAGACGGCATACGA[G^{OXO}]AT-
3′, where [G^{OXO}] = 8-oxoguanine.

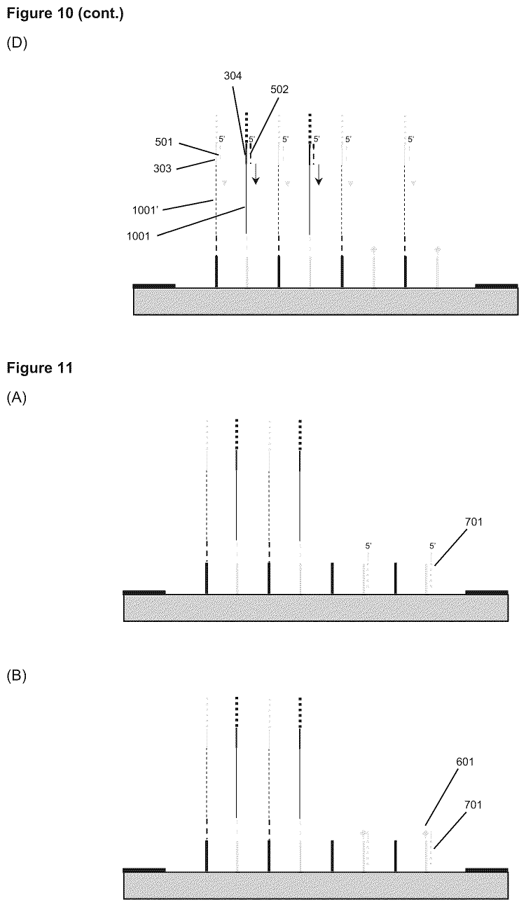

One example of this method is shown in FIG. 9. Selective amplification may be conducted on the amplified (duoclonal) cluster as shown in FIG. 6E. The solid support 200 comprises free first immobilised primers 201 and free second immobilised primers 202 (FIG. 9A). For simplicity, strand 1001′ represents second insert complement sequence 402′, loop complement sequence 403′ and first insert complement sequence 401′, whilst strand 1001 represents first insert sequence 401, loop sequence 403 and second insert sequence 402. Free second immobilised primers 202 are cleaved from the solid support 200, thus leaving behind free first immobilised primers 201 (FIG. 9B).

The first primer-binding sequence 301′ (e.g. P5′) on one set of template strands may then anneal to the free first immobilised primers 201 (e.g. P5 lawn primer) located within the well 203. By contrast, since free second immobilised primers 202 (e.g. P7 lawn primer) have been removed, second primer-binding sequences 302′ (e.g. P7′) are not able to anneal (FIG. 9C).

After conducting a cycle of bridge amplification, this leads to selective amplification of the strand 1001′, relative to the strand 1001 (FIG. 9D).

Conducting standard (non-selective) sequencing then allows strands 1001′ and strands 1001 to be sequenced, wherein a greater proportion of strands 1001′ are sequenced (grey arrow) compared to a proportion of strands 1001 (black arrow) (FIG. 9E).

In another example, selectively processing comprises selectively blocking the extension of some or substantially all of the second immobilised primers that have not yet been extended (extended to form a second polynucleotide strand). Again, these primers may be referred to herein as free or un-extended second immobilised primers. The method may involve using a primer-blocking agent, wherein the primer-blocking agent is configured to limit or prevent synthesis of a strand (i.e. a polynucleotide strand) extending from the second immobilised primer. The method may further involve conducting at least one further amplification cycle. As the free second immobilised primers are blocked from being extended by the primer-blocking agent, only the first immobilised primers can be extended. This leads to amplification of only the first polynucleotide strand (i.e. not the second polynucleotide strand), and as a consequence, an increase in the amount of first polynucleotide sequences relative to the second polynucleotide sequences.

By “some or substantially all” is meant that at least 75%, at least 80%, at least 90% or between 95% and 100% of free second immobilised primers are blocked.

The primer-blocking agent may be flowed across the solid support following bridge amplification. In one embodiment, the primer-blocking agent is flowed across the solid support following at least 1, 2, 3, 4, 5, 6, 7, 8, 9 or 10 cycles, following at least 15, following at least 20 or following at least 25 rounds of bridge amplification.

In one example, the primer-blocking agent is added whilst first polynucleotide sequence(s) are hybridised to the second immobilised primers. That is, the primer-blocking agent is added during amplification and following extension of at least the first polynucleotide strand. At this stage the extended first polynucleotide strand bends (bridges) and hybridises at its 5′ end to the second immobilised primer. Addition of the primer-blocking agent at this stage prevents extension of the second immobilised primer, which would normally occur using the first polynucleotide strand as its template.

In one embodiment, the primer-blocking agent is a blocked nucleotide. In one example, the blocked nucleotide may be A, C, T or G, but may be selected from A or G.

Again, by “blocked” is meant that the sequencing primer comprises a blocking group at a 3′ end of the sequencing primer. Suitable blocking groups include a hairpin loop (e.g. a polynucleotide attached to the 3′-end, comprising in a 5′ to 3′ direction, a cleavable site such as a nucleotide comprising uracil, a loop portion, and a complement portion, wherein the complement portion is substantially complementary to all or a portion of the immobilised primer), a deoxynucleotide, a deoxyribonucleotide, a hydrogen atom instead of a 3′—OH group, a phosphate group, a phosphorothioate group, a propyl spacer (e.g. —O—(CH₂)₃—OH instead of a 3′—OH group)), a modification blocking the 3′-hydroxyl group (e.g. hydroxyl protecting groups, such as silyl ether groups (e.g. trimethylsilyl, triethylsilyl, triisopropylsilyl, t-butyl(dimethyl)silyl, t-butyl(diphenyl)silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl)), or an inverted nucleobase. However, the blocking group may be any modification that prevents extension (i.e. elongation) of the primer by a polymerase. The block may be reversible or irreversible.

The blocked nucleotide may be added as part of a mixture comprising both blocked and unblocked nucleotides. Alternatively, the blocked nucleotide may be added to the flow cell separately and either before or after unblocked nucleotides are added. Following addition of the blocked nucleotide, at least one more round of bridge amplification is performed.

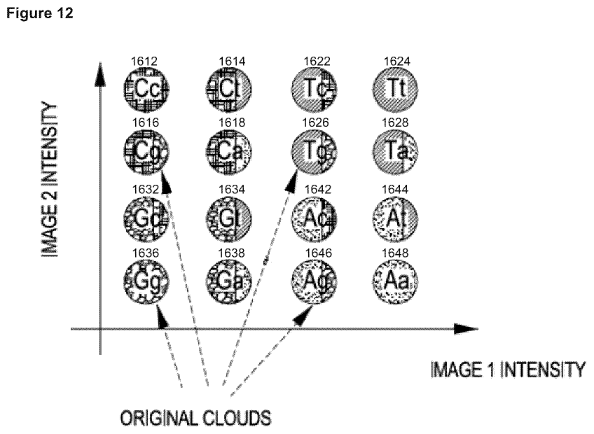

One example of this method is shown in FIG. 10. Selective amplification may be conducted on the amplified (duoclonal) cluster as shown in FIG. 9A. The first primer-binding sequence 301′ (e.g. P5′) on one set of template strands may anneal to first immobilised primers 201 (e.g. P5 lawn primer), and the second primer-binding sequence 302′ (e.g. P7′) on another set of template strands may anneal to second immobilised primers 202 (e.g. P7 lawn primer) (FIG. 10A).

Whilst the second primer-binding sequence 302′ (e.g. P7′) is annealed to the second immobilised primer 202, a primer-blocking agent 601 is selectively installed onto a 3′-end of the second immobilised primer 202, whilst no installation occurs to the 3′-end of the first immobilised primer 201 (FIG. 10B).

After conducting a cycle of bridge amplification, this leads to selective amplification of the strands 1001′, relative to the strands 1001. The primer-blocking agent 601 prevents extension from the second immobilised primer 202 (FIG. 10C).

Conducting standard (non-selective) sequencing then allows strands 1001′ and strands 1001 to be sequenced, wherein a greater proportion of strands 1001′ are sequenced (grey arrow) compared to a proportion of strands 1001 (black arrow) (FIG. 10D).

In an alternative example, the method comprises flowing at least one or a plurality of, extended primer sequence(s) across the surface of the solid support (e.g. a flow cell), wherein such sequences can bind (e.g. hybridise) free immobilised primers (e.g. P5 or P7) and wherein the extended primer sequences further comprise at least one 5′ additional nucleotide; and (b) adding the primer blocking agent, where the primer blocking agent is complementary to the 5′ additional nucleotide.

In one embodiment, the extended primer sequences are substantially complementary to the first or second immobilised primers (e.g. P5 or P7), or substantially complementary to a portion of the first or second immobilised primer.

The 5′ additional nucleotide may be selected from A, T, C or G, but may be T (or U) or C. In one embodiment, the 5′ additional nucleotide is not a complement of the 3′ nucleotide of the second immobilised primer (where the extended primer sequence binds the first immobilised primer) or is not a complement of the 3′ nucleotide of the first immobilised primer (where the extended primer sequence binds the second immobilised primer). For example, where the first immobilised primer is P5 (for example as defined in SEQ ID NO: 1) and the second immobilised primer is P7 for example as defined in SEQ ID NO: 2), and where the extended primer sequence binds the first immobilised primer, the 5′ additional nucleotide is not A. Similarly, where the extended primer sequence binds the second immobilised primer, the 5′ additional nucleotide is not G.

In one embodiment, the primer-blocking agent is a blocked nucleotide, for example, as described above. In one embodiment, the blocked nucleotide may be A, C, T or G, but may be selected from A or G. Accordingly, where the 5′ additional nucleotide is T or U, the primer-blocking agent is A, and where the 5′ additional nucleotide is C, the primer-blocking agent is G.

Again, the extended primer sequence(s) and primer-blocking agent may be flowed across the solid support following bridge amplification. In one embodiment, the primer-blocking agent is flowed across the solid support following at least 1, at least 2, at least 3, at least 4, at least 5, at least 6, at least 7, at least 8, at least 9, at least 10, at least 15, at least 20 or following at least 25 rounds of bridge amplification.

In one embodiment, the extended primer sequence is selected from SEQ ID NO: 55 to 66 or a variant or fragment thereof.

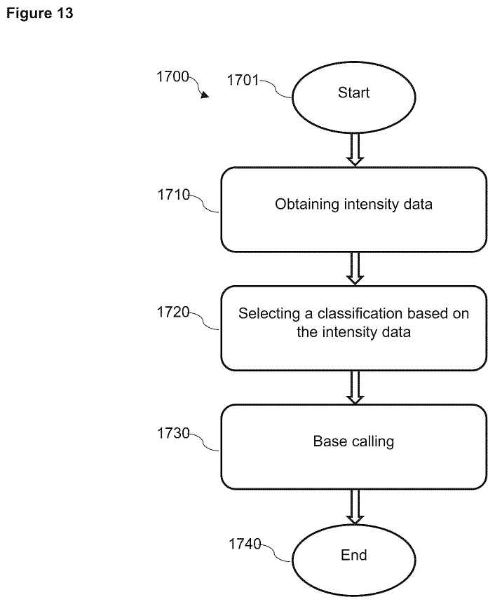

One example of this method is shown in FIG. 11. Selective amplification may be conducted on the amplified (duoclonal) cluster as shown in FIG. 9A; as such following a number of rounds of amplification, a cluster is formed comprising both extended first (e.g. P5) and second (e.g. P7) immobilised polynucleotide strands. Before the next round of amplification, a (or a plurality of) extended primer sequence(s) is flowed across the surface of the solid support 200. The extended primer sequence 701 is substantially complementary to at least a portion, if not all of the immobilised primer (e.g. either P5 or P7) and binds to the immobilised primer (e.g. P5 or P7) as shown in FIG. 11A. As also shown in FIG. 11A, the extended primer sequence 701 comprises at least one additional 5′ nucleotide.

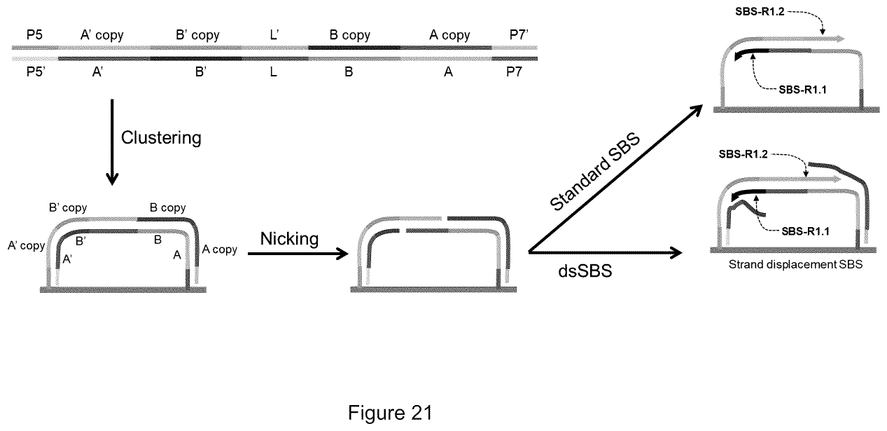

Following addition of the extended primer sequence 701, a primer blocking agent 601 is added and flowed across the surface of the solid support (e.g. flow cell). As the primer-blocking agent 601 is complementary to the 5′ additional nucleotide of the extended primer sequence 701 the primer-blocking agent 601 binds to the 3′-end of the immobilised strands that are hybridised to the extended primer sequence 701, as shown in FIG. 16B. As a consequence, addition of the primer-blocking agent 601 prevents not only extension of the immobilised strand (e.g. P5 or P7) but renders the immobilised primer (P5 or P7) unavailable for hybridisation and subsequent bridge amplification for other extended strands (e.g. 101′) (see FIG. 11B).

Performing at least one more cycle of bridge amplification, leads to selective amplification of strands 1001′ (in a 2:1 ratio of 1001′ to 1001). Again, similar to FIG. 10D, conducting standard (non-selective) sequencing then allows strands 1001′ and strands 1001 to be sequenced, wherein a greater proportion of strands 1001′ are sequenced (grey arrow) compared to a proportion of strands 1001 (black arrow) (FIG. 10D).

The extended primer sequences may be added as part of the amplification mixture described above. Alternatively, the blocked immobilised primer-binding sequence may be added to the flow cell separately and may be before the amplification mixture is added. Following addition of the blocked immobilised primer-binding sequence, at least one more round of bridge amplification is performed.

### Data Analysis Using 16 QaM

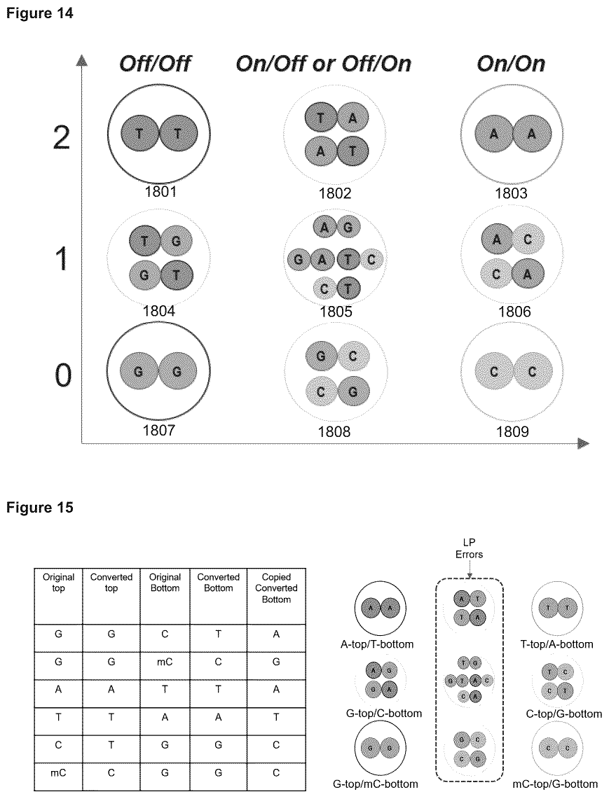

FIG. 12 is a scatter plot showing an example of sixteen distributions of signals generated by polynucleotide sequences disclosed herein.

The scatter plot of FIG. 12 shows sixteen distributions (or bins) of intensity values from the combination of a brighter signal (i.e. a first signal as described herein) and a dimmer signal (i.e. a second signal as described herein); the two signals may be co-localized and may not be optically resolved as described above. The intensity values shown in FIG. 12 may be up to a scale or normalisation factor; the units of the intensity values may be arbitrary or relative (i.e., representing the ratio of the actual intensity to a reference intensity). The sum of the brighter signal generated by the first portions and the dimmer signal generated by the second portions results in a combined signal. The combined signal may be captured by a first optical channel and a second optical channel. Since the brighter signal may be A, T, C or G, and the dimmer signal may be A, T, C or G, there are sixteen possibilities for the combined signal, corresponding to sixteen distinguishable patterns when optically captured. That is, each of the sixteen possibilities corresponds to a bin shown in FIG. 12. The computer system can map the combined signal generated into one of the sixteen bins, and thus determine the added nucleobase at the first portion and the added nucleobase at the second portion, respectively.

For example, when the combined signal is mapped to bin 1612 for a base calling cycle, the computer processor base calls both the added nucleobase at the first portion and the added nucleobase at the second portion as C. When the combined signal is mapped to bin 1614 for the base calling cycle, the processor base calls the added nucleobase at the first portion as C and the added nucleobase at the second portion as T. When the combined signal is mapped to bin 1616 for the base calling cycle, the processor base calls the added nucleobase at the first portion as C and the added nucleobase at the second portion as G. When the combined signal is mapped to bin 1618 for the base calling cycle, the processor base calls the added nucleobase at the first portion as C and the added nucleobase at the second portion as A.

When the combined signal is mapped to bin 1622 for the base calling cycle, the processor base calls the added nucleobase at the first portion as T and the added nucleobase at the second portion as C. When the combined signal is mapped to bin 1624 for the base calling cycle, the processor base calls both the added nucleobase at the first portion and the added nucleobase at the second portion as T. When the combined signal is mapped to bin 1626 for the base calling cycle, the processor base calls the added nucleobase at the first portion as T and the added nucleobase at the second portion as G. When the combined signal is mapped to bin 1628 for the base calling cycle, the processor base calls the added nucleobase at the first portion as T and the added nucleobase at the second portion as A.

When the combined signal is mapped to bin 1632 for the base calling cycle, the processor base calls the added nucleobase at the first portion as G and the added nucleobase at the second portion as C. When the combined signal is mapped to bin 1634 for the base calling cycle, the processor base calls the added nucleobase at the first portion as G and the added nucleobase at the second portion as T. When the combined signal is mapped to bin 1636 for the base calling cycle, the processor base calls both the added nucleobase at the first portion and the added nucleobase at the second portion as G. When the combined signal is mapped to bin 1638 for the base calling cycle, the processor base calls the added nucleobase at the first portion as G and the added nucleobase at the second portion as A.

When the combined signal is mapped to bin 1642 for the base calling cycle, the processor base calls the added nucleobase at the first portion as A and the added nucleobase at the second portion as C. When the combined signal is mapped to bin 1644 for the base calling cycle, the processor base calls the added nucleobase at the first portion as A and the added nucleobase at the second portion as T. When the combined signal is mapped to bin 1646 for the base calling cycle, the processor base calls the added nucleobase at the first portion as A and the added nucleobase at the second portion as G. When the combined signal is mapped to bin 1648 for the base calling cycle, the processor base calls both the added nucleobase at the first portion and the added nucleobase at the second portion as A.

In this particular example, T is configured to emit a signal in both the IMAGE 1 channel and the IMAGE 2 channel, A is configured to emit a signal in the IMAGE 1 channel only, C is configured to emit a signal in the IMAGE 2 channel only, and G does not emit a signal in either channel. However, different permutations of nucleobases can be used to achieve the same effect by performing dye swaps. For example, A may be configured to emit a signal in both the IMAGE 1 channel and the IMAGE 2 channel, T may be configured to emit a signal in the IMAGE 1 channel only, C may be configured to emit a signal in the IMAGE 2 channel only, and G may be configured to not emit a signal in either channel.

Further details regarding performing base-calling based on a scatter plot having sixteen bins may be found in U.S. Patent Application Publication No. 2019/0212294, the disclosure of which is incorporated herein by reference.

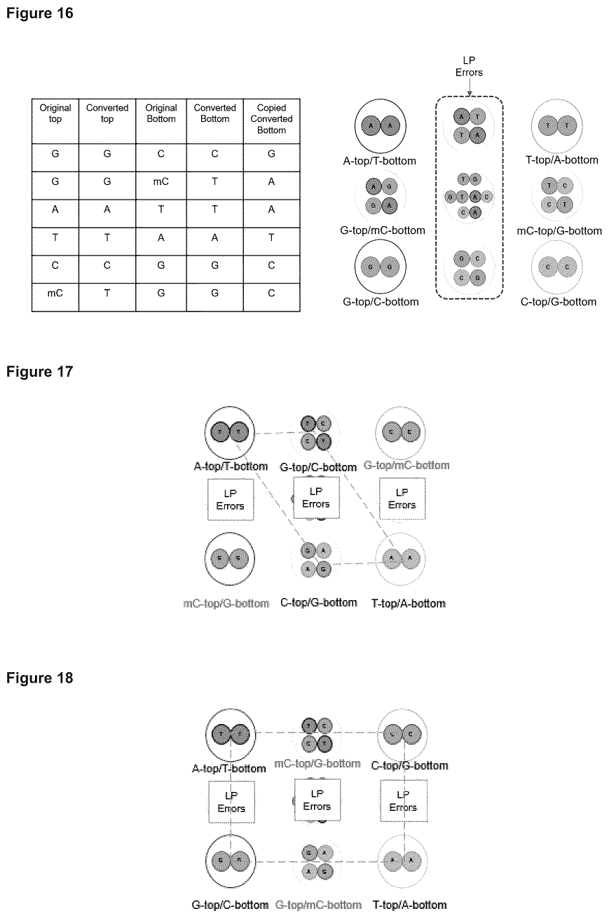

FIG. 13 is a flow diagram showing a method 1700 of base calling according to the present disclosure. The described method allows for simultaneous sequencing of two (or more) portions (e.g. the first portion and the second portion) in a single sequencing run from a single combined signal obtained from the first portion and the second portion, thus requiring less sequencing reagent consumption and faster generation of data from both the first portion and the second portion. Further, the simplified method may reduce the number of workflow steps while producing the same yield as compared to existing next-generation sequencing methods. Thus, the simplified method may result in reduced sequencing runtime.

As shown in FIG. 13, the disclosed method 1700 may start from block 1701. The method may then move to block 1710.

At block 1710, intensity data is obtained. The intensity data includes first intensity data and second intensity data. The first intensity data comprises a combined intensity of a first signal component obtained based upon a respective first nucleobase of the first portion and a second signal component obtained based upon a respective second nucleobase of the second portion. Similarly, the second intensity data comprises a combined intensity of a third signal component obtained based upon the respective first nucleobase of the first portion and a fourth signal component obtained based upon the respective second nucleobase of the second portion.

As such, the first portion is capable of generating a first signal comprising a first signal component and a third signal component. The second portion is capable of generating a second signal comprising a second signal component and a fourth signal component.

As described above, the first portion and the second portion may be arranged on the solid support such that signals from the first portion and the second portion are detected by a single sensing portion and/or may comprise a single cluster such that first signals and second signals from each of the respective first portions and second portions cannot be spatially resolved.

In one example, obtaining the intensity data comprises selecting intensity data that corresponds to two (or more) different portions (e.g. the first portion and the second portion). In one example, intensity data is selected based upon a chastity score. A chastity score may be calculated as the ratio of the brightest base intensity divided by the sum of the brightest and second brightest base intensities. The desired chastity score may be different depending upon the expected intensity ratio of the light emissions associated with the different portions. As described above, it may be desired to produce clusters comprising the first portion and the second portion, which give rise to signals in a ratio of 2:1. In one example, high-quality data corresponding to two portions with an intensity ratio of 2:1 may have a chastity score of around 0.8 to 0.9.

After the intensity data has been obtained, the method may proceed to block 1720. In this step, one of a plurality of classifications is selected based on the intensity data. Each classification represents a possible combination of respective first and second nucleobases. In one example, the plurality of classifications comprises sixteen classifications as shown in FIG. 12, each representing a unique combination of first and second nucleobases. Where there are two portions, there are sixteen possible combinations of first and second nucleobases. Selecting the classification based on the first and second intensity data comprises selecting the classification based on the combined intensity of the first and second signal components and the combined intensity of the third and fourth signal components.

The method may then proceed to block 1730, where the respective first and second nucleobases are base called based on the classification selected in block 1720. The signals generated during a cycle of a sequencing are indicative of the identity of the nucleobase(s) added during sequencing (e.g. using sequencing-by-synthesis). It will be appreciated that there is a direct correspondence between the identity of the nucleobases that are incorporated and the identity of the complementary base at the corresponding position of the template sequence bound to the solid support. Therefore, any references herein to the base calling of respective nucleobases at the two portions encompasses the base calling of nucleobases hybridised to the template sequences and, alternatively or additionally, the identification of the corresponding nucleobases of the template sequences. The method may then end at block 1740.

### Data Analysis Using 9 QaM

For two portions of polynucleotide sequences (e.g. a first portion and a second portion as described herein), there are sixteen possible combinations of nucleobases at any given position (i.e., an A in the first portion and an A in the second portion, an A in the first portion and a T in the second portion, and so on). When the same nucleobase is present at a given position in both portions, the light emissions associated with each target sequence during the relevant base calling cycle will be characteristic of the same nucleobase. In effect, the two portions behave as a single portion, and the identity of the bases at that position are uniquely callable.

However, when a nucleobase of the first portion is different from a nucleobase at a corresponding position of the second portion, the signals associated with each portion in the relevant base calling cycle will be characteristic of different nucleobases. In one embodiment, the first signal coming from the first portion have substantially the same intensity as the second signal coming from the second portion. The two signals may also be co-localised, and may not be spatially and/or optically resolved. Therefore, when different nucleobases are present at corresponding positions of the two portions, the identity of the nucleobases cannot be uniquely called from the combined signal alone. However, useful sequencing information can still be determined from these signals.

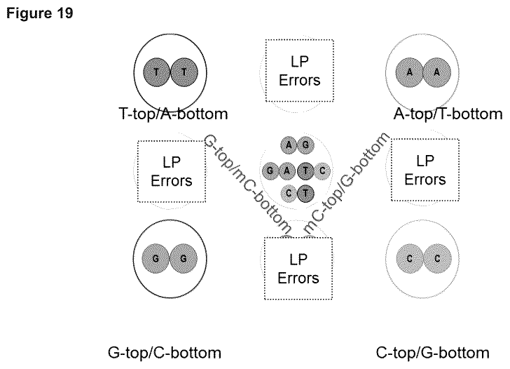

The scatter plot of FIG. 14 shows nine distributions (or bins) of intensity values from the combination of two co-localised signals of substantially equal intensity.

The intensity values shown in FIG. 14 may be up to a scale or normalisation factor; the units of the intensity values may be arbitrary or relative (i.e., representing the ratio of the actual intensity to a reference intensity). The sum of the first signal generated from the first portion and the second signal generated from the second portion results in a combined signal. The combined signal may be captured by a first optical channel and a second optical channel. The computer system can map the combined signal generated into one of the nine bins, and thus determine sequence information relating to the added nucleobase at the first portion and the added nucleobase at the second portion.

Bins are selected based upon the combined intensity of the signals originating from each target sequence during the base calling cycle. For example, bin 1803 may be selected following the detection of a high-intensity (or “on/on”) signal in the first channel and a high-intensity signal in the second channel. Bin 1806 may be selected following the detection of a high-intensity signal in the first channel and an intermediate-intensity (“on/off” or “off/on”) signal in the second channel. Bin 1809 may be selected following the detection of a high-intensity signal in the first channel and a low-intensity or zero-intensity (“off/off”) signal in the second channel. Bin 1802 may be selected following the detection of an intermediate-intensity signal in the first channel and a high-intensity signal in the second channel. Bin 1805 may be selected following the detection of an intermediate-intensity signal in the first channel and an intermediate-intensity signal in the second channel. Bin 1808 may be selected following the detection of an intermediate-intensity signal in the first channel and a low-intensity or zero-intensity signal in the second channel. Bin 1801 may be selected following the detection of a low-intensity signal in the first channel and a high-intensity signal in the second channel. Bin 1804 may be selected following the detection of a low-intensity or zero-intensity signal in the first channel and an intermediate-intensity signal in the second channel. Bin 1807 may be selected following the detection of a low-intensity or zero-intensity signal in the first channel and a low-intensity signal in the second channel.

Four of the nine bins represent matches between respective nucleobases of the two portions sensed during the cycle (bins 1801, 1803, 1807, and 1809). In response to mapping the combined signal to a bin representing a match, the computer processor may detect a match between the first portion and the second portion at the sensed position. In response to mapping the combined signal to a bin representing a match, the computer processor may base call the respective nucleobases. For example, when the combined signal is mapped to bin 1801 for a base calling cycle, the computer processor base calls both the added nucleobase at the first portion and the added nucleobase at the second portion as T. When the combined signal is mapped to bin 1803 for the base calling cycle, the processor base calls both the added nucleobase at the first portion and the added nucleobase at the second portion as A. When the combined signal is mapped to bin 1807 for the base calling cycle, the processor base calls both the added nucleobase at the first portion and the added nucleobase at the second portion as G. When the combined signal is mapped to bin 1809 for the base calling cycle, the processor base calls both the added nucleobase at the first portion and the added nucleobase at the second portion as C.

The remaining five bins are “ambiguous”. That is to say that these bins each represent more than one possible combination of first and second nucleobases. Bins 1802, 1804, 1806, and 1808 each represent two possible combinations of first and second nucleobases. Bin 1805, meanwhile, represents four possible combinations. Nevertheless, mapping the combined signal to an ambiguous bin may still allow for sequencing information to be determined. For example, bins 1802, 1804, 1805, 1806, and 1808 represent mismatches between respective nucleobases of the two portions sensed during the cycle. Therefore, in response to mapping the combined signal to a bin representing a mismatch, the computer processor may detect a mismatch between the first portion and the second portion at the sensed position.

The remaining five bins are “ambiguous”. That is to say that these bins each represent more than one possible combination of first and second nucleobases. Bins 1802, 1804, 1806, and 1808 each represent two possible combinations of first and second nucleobases. Bin 1805, meanwhile, represents four possible combinations.

Nevertheless, mapping the combined signal to an ambiguous bin may still allow for sequencing information to be determined. For example, bins 1802, 1804, 1805, 1806, and 1808 represent mismatches between respective nucleobases of the two portions sensed during the cycle. Therefore, in response to mapping the combined signal to a bin representing a mismatch, the computer processor may detect a mismatch between the first portion and the second portion at the sensed position.

In this particular example, A is configured to emit a signal in both the first channel and the second channel, C is configured to emit a signal in the first channel only, T is configured to emit a signal in the second channel only, and G does not emit a signal in either channel. However, different permutations of nucleobases can be used to achieve the same effect by performing dye swaps. For example, A may be configured to emit a signal in both the first channel and the second channel, T may be configured to emit a signal in the first channel only, C may be configured to emit a signal in the second channel only, and G may be configured to not emit a signal in either channel.

The number of classifications which may be selected based upon the combined signal intensities may be predetermined, for example based on the number of portions expected to be present in the nucleic acid cluster. Whilst FIG. 14 shows a set of nine possible classifications, the number of classifications may be greater or smaller.

In addition to identifying matches and mismatches, the mapping of the combined signal to each of the different bins (e.g. in combination with additional knowledge, such as the library preparation methods used) can provide additional information about the first portion and the second portion, or about sequences from which the first portion and the second portion were derived. For example, given the nucleic acid material input and the processing methods used to generate the nucleic acid clusters, the first portion and the second portion may be expected to be identical at a given position. In this case, the mapping of the combined signal to a bin representing a mismatch may be indicative of an error introduced during library preparation. In addition, the first portion and the second portion may be expected to be different, for example due to deliberate sequence modifications introduced during library preparation to detect modified cytosines.

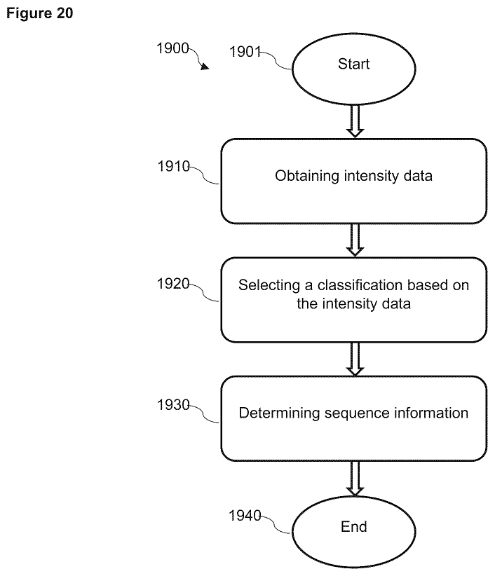

As mentioned herein, the library preparation may involve treatment with a conversion agent. In cases where the conversion reagent is configured to convert an unmodified cytosine to uracil or a nucleobase which is read as thymine/uracil, the correspondence between bases in the original polynucleotide and in the converted strands is shown in FIG. 15, alongside a scatter plot showing potential resulting distributions for the combined signal intensities resulting from the simultaneous sequencing of the target sequences. An A-T or T-A base pair in the original molecule will result in a match (A/A or T/T) at the corresponding position of the forward and reverse complement strands of the library. An mC-G or G-mC base pair in the library will also result in a match (G/G or C/C) at the corresponding position of the forward and reverse complement strands of the library. For a C-G base pair, however, the conversion of unmodified cytosine to uracil (or a nucleobase which is read as thymine/uracil) in the forward strand of the library (“top” strand) will result in a T at the corresponding position of the forward strand of the library. Meanwhile, the corresponding position on the reverse complement strand of the library (“bottom” strand) will be occupied by C. Alternatively, for a G-C base pair, the conversion of unmodified cytosine to uracil (or a nucleobase which is read as thymine/uracil) in the reverse strand of the library (“bottom” strand) will result in an A at the corresponding position of the reverse complement strand of the library. Meanwhile, the corresponding position of the forward strand of the library (“top” strand) will be occupied by G. Therefore, in response to mapping the combined signal to the distribution representing G/G or C/C, the presence of a modified cytosine can be determined at the corresponding position in the original polynucleotide.

In other cases where the conversion reagent is configured to convert a modified cytosine to thymine or a nucleobase which is read as thymine/uracil, FIG. 16 shows the correspondence between bases in the original polynucleotide and in the converted strands, alongside a scatter plot showing potential resulting distributions for the combined signal intensities resulting from the simultaneous sequencing of the target sequences. An A-T or T-A base pair in the library will result in a match (A/A or T/T) at the corresponding position of the forward and reverse complement strands of the library. A C-G or G-C base pair in the library will also result in a match (G/G or C/C) at the corresponding position of the forward and reverse complement strands of the library. For a mC-G base pair, however, the conversion of 5-methylcytosine to thymine in the forward strand of the library (“top” strand) will result in a T at the corresponding position of the forward strand of the library. Meanwhile, the corresponding position on the reverse complement strand of the library (“bottom” strand) will be occupied by C. Alternatively, the conversion of 5-methylcytosine to thymine in the reverse strand of the library (“bottom” strand) will result in an A at the corresponding position of the reverse complement strand of the library. Meanwhile, the corresponding position of the forward strand of the library (“top” strand) will be occupied by G. Therefore, in response to mapping the combined signal to the distribution representing an A/G, G/A, T/C, or C/T mismatch, the presence of a modified cytosine can be determined at the corresponding position in the original polynucleotide.

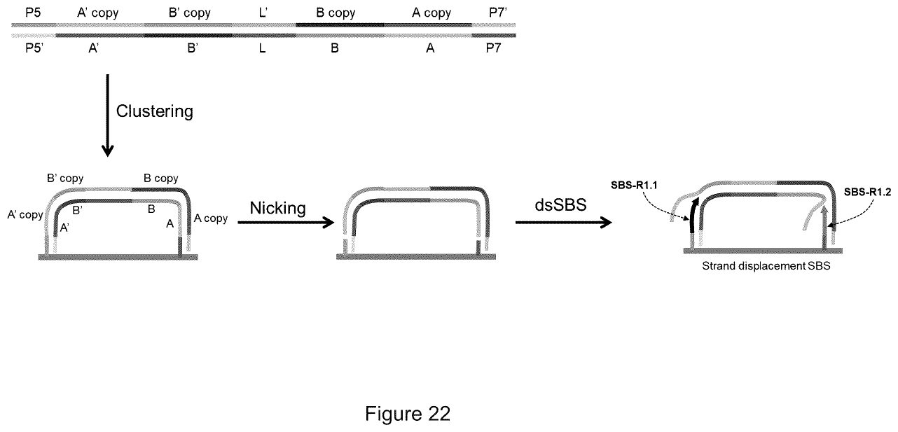

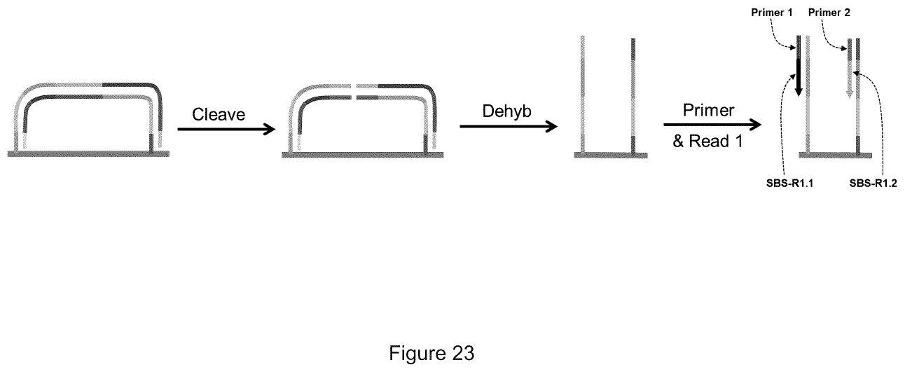

FIG. 17 represents the distributions resulting from the use of an alternative dye-encoding scheme following use of a conversion reagent configured to convert an unmodified cytosine to uracil or a nucleobase which is read as thymine/uracil, and FIG. 18 represents the distributions resulting from the use of an alternative dye-encoding scheme following use of a conversion reagent configured to convert a modified cytosine to thymine or a nucleobase which is read as thymine/uracil.

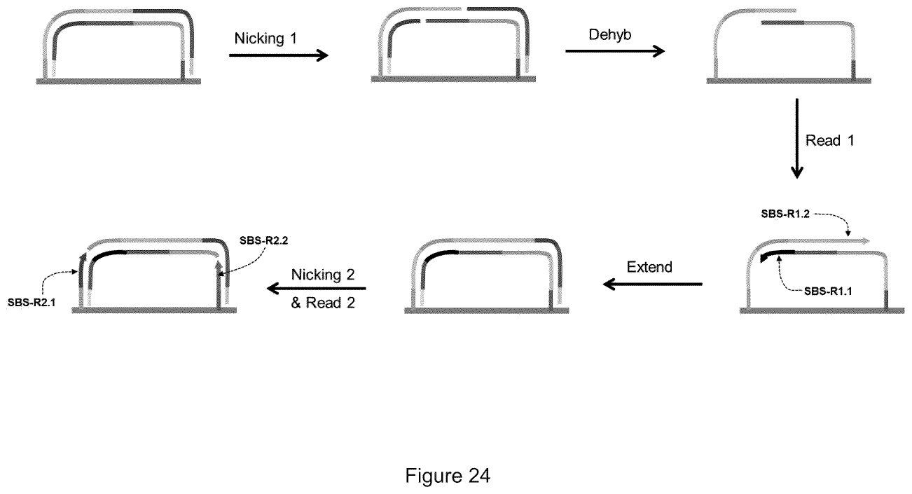

FIG. 19 represents yet another distribution resulting from the use of an alternative dye-encoding scheme following use of a conversion reagent configured to convert a modified cytosine to thymine or a nucleobase which is read as thymine/uracil. In this case, modified cytosines fall within a central bin.

In the present example, for each base pair in the original double-stranded DNA molecule, it may be assumed that there are six possibilities: A-T, T-A, C-G, G-C, mC-G and G-mC. As shown in FIGS. 15 to 18, each of these possibilities is uniquely represented by one of the plurality of classifications. According to the present methods, it is therefore possible to determine both the sequence and “methylation” status (i.e. presence of modified cytosines) of a double-stranded polynucleotide in a single sequencing run.

In addition to determining “methylation” status, it may also be possible to identify library preparation/sequencing errors. Using the dye-encoding scheme shown in FIGS. 15 and 16, the central column of distributions is indicative of such errors. Using the dye encoding scheme shown in FIGS. 17 and 18, the central row of distributions is indicative of such errors.

The dye-encoding scheme may be optimised to allow for different combinations of first and second nucleobases to be resolved. This may be particularly useful where sequence modifications of a known type have been introduced into the first portions and the second portions. For example, where sequence modifications have been introduced that result in the conversion of unmodified cytosines to uracil or nucleobases which is read as thymine/uracil, or the conversion of modified cytosines to thymine or nucleobases which are read as thymine/uracil, the dye-encoding scheme may be selected such that the resulting combination of first and second nucleobases do not fall within the central bin (which represents four different nucleobase combinations).

In the case of conversion of modified cytosines to thymine (or nucleobases which are read as thymine/uracil), a T/C or G/A mismatch between the forward and reverse complement strands is indicative of the presence of a mC-G or G-mC base pair at the corresponding position of the library. The dye-encoding scheme may therefore be designed such that these mismatches may be resolved from other possible combinations of nucleobases. This may be achieved by detecting light emissions from A and T bases in a first illumination cycle, and from C and T bases in a second illumination cycle. In another example, light emissions may be detected from C and G bases in a first illumination cycle, and from C and T bases in a second illumination cycle. In another example, light emissions may be detected from C and A bases in a first illumination cycle, and from C and G bases in a second illumination cycle.

In the case of unmodified cytosines to uracil (or nucleobases which is read as thymine/uracil), a C/C or G/G match between the forward and reverse complement strands is indicative of the presence of a mC-G or G-mC base pair at the corresponding position of the library. In this case, a mC-G or G-mC base pair will always be resolvable. However, the dye-encoding scheme can still be designed to optimise the resolution between unmodified bases.

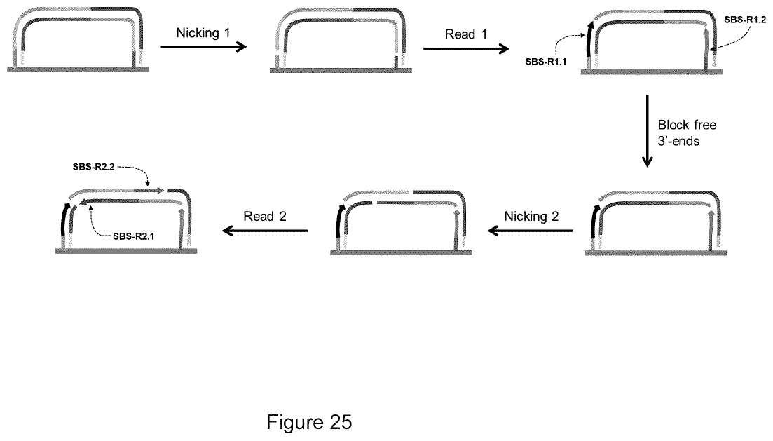

FIG. 20 is a flow diagram showing a method 1900 of determining sequence information according to the present disclosure. The described method allows for the determination of sequence information from two (or more) portions (e.g. the first portion and the second portion) in a single sequencing run from a single combined signal obtained from the first portion and the second portion.

As shown in FIG. 20, the disclosed method 1900 may start from block 1901. The method may then move to block 1910.

At block 1910, intensity data is obtained. The intensity data includes first intensity data and second intensity data. The first intensity data comprises a combined intensity of a first signal component obtained based upon a respective first nucleobase of the first portion and a second signal component obtained based upon a respective second nucleobase of the second portion. Similarly, the second intensity data comprises a combined intensity of a third signal component obtained based upon the respective first nucleobase of the first portion and a fourth signal component obtained based upon the respective second nucleobase of the second portion.

As such, the first portion is capable of generating a first signal comprising a first signal component and a third signal component. The second portion is capable of generating a second signal comprising a second signal component and a fourth signal component.

As described above, the first portion and the second portion may be arranged on the solid support such that signals from the first portion and the second portion are detected by a single sensing portion and/or may comprise a single cluster such that first signals and second signals from each of the respective first portions and second portions cannot be spatially resolved.

In one example, obtaining the intensity data comprises selecting intensity data, for example based upon a chastity score. A chastity score may be calculated as the ratio of the brightest base intensity divided by the sum of the brightest and second brightest base intensities. In one example, high-quality data corresponding to two portions with a substantially equal intensity ratio may have a chastity score of around 0.8 to 0.9, for example 0.89-0.9.

After the intensity data has been obtained, the method may proceed to block 1920. In this step, one of a plurality of classifications is selected based on the intensity data. Each classification represents one or more possible combinations of respective first and second nucleobases, and at least one classification of the plurality of classifications represents more than one possible combination of respective first and second nucleobases. In one example, the plurality of classifications comprises nine classifications as shown in FIG. 14. Selecting the classification based on the first and second intensity data comprises selecting the classification based on the combined intensity of the first and second signal components and the combined intensity of the third and fourth signal components.

The method may then proceed to block 1930, where sequence information of the respective first and second nucleobases is determined based on the classification selected in block 1920. The signals generated during a cycle of a sequencing are indicative of the identity of the nucleobase(s) added during sequencing (e.g. using sequencing-by-synthesis). For example, it may be determined that there is a match or a mismatch between the respective first and second nucleobases. Where it is determined that there is a match between the first and second respective nucleobases, the nucleobases may be base called. Whether there is a match or a mismatch, additional or alternative information may be obtained, as described above. It will be appreciated that there is a direct correspondence between the identity of the nucleobases that are incorporated and the identity of the complementary base at the corresponding position of the template sequence bound to the solid support. Therefore, any references herein to the base calling of respective nucleobases at the two portions encompasses the base calling of nucleobases hybridised to the template sequences and, alternatively or additionally, the identification of the corresponding nucleobases of the template sequences. The method may then end at block 1940.

### Sequencing of Modified Cytosines

The present invention is directed to methods of preparing polynucleotide strands for detection of modified cytosines, such that where the strands comprises two portions (in other words, separate polynucleotide sequences, where a first polynucleotide sequence comprises a first portion and a second polynucleotide sequence comprises a second portion) to be identified, the first portion comprising a forward strand and the second portion comprising a reverse complement strand (or the first portion comprising a reverse strand and the second portion comprising a forward complement strand), such portions can be identified concurrently, thus facilitating the detection of modified cytosines.

Advantageously, the methods of the present invention allow a decrease in the amount of time taken to detect modified cytosines.

In some embodiments, selective processing methods may be used when preparing the templates. This leads to further advantages, as it also becomes possible to identify which strand of the original library that the modified cytosine was on, whilst maintaining reductions in time taken to detect modified cytosines.

Accordingly, we describe a method of preparing polynucleotide sequences for detection of modified cytosines, comprising:
synthesising at least one first polynucleotide sequence comprising a first portion and at least one second polynucleotide sequence comprising a second portion,
wherein the at least one first polynucleotide sequence comprising a first portion and the at least one second polynucleotide sequence comprising a second portion each comprise portions of a double-stranded nucleic acid template, and the first portion comprises a forward strand of the template, and the second portion comprises a reverse complement strand of the template; or wherein the first portion comprises a reverse strand of the template, and the second portion comprises a forward complement strand of the template,
wherein the template is generated from a (double-stranded) target polynucleotide to be sequenced via complementary base pairing, and wherein the target polynucleotide has been pre-treated using a conversion reagent,
wherein the conversion reagent is configured to convert a modified cytosine to thymine or a nucleobase which is read as thymine/uracil, and/or wherein the conversion reagent is configured to convert an unmodified cytosine to uracil or a nucleobase which is read as thymine/uracil.

As described herein, the polynucleotide sequences each comprise portions of a double-stranded nucleic acid template, and the first portion may comprise (or be) the forward strand of a polynucleotide sequence (e.g. forward strand of a template), and the second portion may comprise (or be) the reverse complement strand of the polynucleotide sequence (e.g. reverse complement strand of the template) (in effect, a reverse complement strand may be considered a “copy” of the forward strand). Alternatively, the first portion may comprise (or be) the reverse strand of a polynucleotide sequence (e.g. reverse strand of a template), and the second portion may comprise (or be) the forward complement strand of the polynucleotide sequence (e.g. forward complement strand of the template) (in effect, a forward complement may be considered a “copy” of the reverse strand).

The first portion may be derived from a forward strand of a target polynucleotide to be sequenced, and the second portion may be derived from a reverse complement strand of the target polynucleotide to be sequenced; or the first portion may be derived from a reverse strand of a target polynucleotide to be sequenced, and the second portion may be derived from a forward complement strand of the target polynucleotide to be sequenced.

The template is generated from a (double-stranded) target polynucleotide to be sequenced via complementary base pairing. The (double-stranded) target polynucleotide may be one (double-stranded) polynucleotide present in a polynucleotide library to be sequenced. As such, the template allows sequence information to be obtained for that particular polynucleotide.

The method may further comprise a step of preparing the first portion and the second portion for concurrent sequencing.

For example, the method may comprise simultaneously contacting first sequencing primer binding sites located after a 3′-end of the first portions with first primers and second sequencing primer binding sites located after a 3′-end of the second portions with second primers. Thus, the first portions and second portions are primed for concurrent sequencing.

The method may alternatively or additionally comprise nicking the at least one first polynucleotide sequence and nicking the at least one second polynucleotide sequence.

In some embodiments, the nick on the at least one first polynucleotide sequence may be located after a 3′-end of the first portion, and the nick on the at least one second polynucleotide sequence may be located after a 3′-end of the second portion. In some embodiments, the nick on the at least one first polynucleotide sequence may be located before a 5′-end of the first portion, and the nick on the at least one second polynucleotide sequence may be located before a 5′-end of the second portion. Thus, the first portions and second portions are primed for concurrent sequencing as sequencing may begin from the nick (e.g. by using strand displacement SBS, or after washing off non-immobilised strands).

In some embodiments, a proportion of first portions may be capable of generating a first signal and a proportion of second portions may be capable of generating a second signal, wherein an intensity of the first signal is substantially the same as an intensity of the second signal.

In other embodiments (e.g. where selective processing methods are used as described herein), a proportion of first portions may be capable of generating a first signal and a proportion of second portions may be capable of generating a second signal, wherein an intensity of the first signal is substantially the same as an intensity of the second signal.

The first signal and the second signal may be spatially unresolved (e.g. generated from the same region or substantially overlapping regions).

Further aspects relating to selective processing methods (e.g. conducting selective sequencing or preparing for selective sequencing) have already been described herein and apply to the methods of preparing polynucleotide sequences for detection of modified cytosines as described herein.

The first portion may be referred to herein as read 1.1 (R1.1). The second portion may be referred to herein as read 1.2 (R1.2).

The first and second strand may be separately attached to a solid support. In one embodiment, this solid support is a flow cell. In another embodiment, each of the first and second strands are attached to the solid support (e.g. flow cell) in a single well of the solid support.

The polynucleotide strands may form or be part of a cluster on the solid support.

As used herein, the term “cluster” may refer to a clonal group of template polynucleotides (e.g. DNA or RNA) bound within a single well of a solid support (e.g. flow cell). As such, a cluster may refer to the population of polynucleotide molecules within a well that are then sequenced. A “cluster” may contain a sufficient number of copies of template polynucleotides such that the cluster is able to output a signal (e.g. a light signal) that allows sequencing reads to be performed on the cluster. A “cluster” may comprise, for example, about 500 to about 2000 copies, about 600 to about 1800 copies, about 700 to about 1600 copies, about 800 to 1400 copies, about 900 to 1200 copies, or about 1000 copies of template polynucleotides.

A cluster may be formed by bridge amplification, as described above.

Where the method of the invention involves a first polynucleotide strand and a second polynucleotide strand, the cluster formed may be a duoclonal cluster.

By “duoclonal” cluster is meant that the population of polynucleotide sequences that are then sequenced (as the next step) are substantially of two types—e.g. a first sequence and a second sequence. As such, a “duoclonal” cluster may refer to the population of single first sequences and single second sequences within a well that are then sequenced. A “duoclonal” cluster may contain a sufficient number of copies of a single first sequence and copies of a single second sequence such that the cluster is able to output a signal (e.g. a light signal) that allows sequencing reads to be performed on the “monoclonal” cluster. A “duoclonal” cluster may comprise, for example, about 500 to about 2000 combined copies, about 600 to about 1800 combined copies, about 700 to about 1600 combined copies, about 800 to 1400 combined copies, about 900 to 1200 combined copies, or about 1000 combined copies of single first sequences and single second sequences. The copies of single first sequences and single second sequences together may comprise at least about 50%, at least about 60%, at least about 70%, at least about 80%, at least about 90%, or about 95%, 98%, 99% or 100% of all polynucleotides within a single well of the flow cell, and thus providing a substantially duoclonal “cluster”.

The at least one first polynucleotide sequence comprising a first portion and at least one second polynucleotide sequence may be prepared using a loop fork method as described herein (see FIG. 4).

Accordingly, in one embodiment, the step of synthesising at least one first polynucleotide sequence comprising a first portion and at least one second polynucleotide sequence comprising a second portion may comprise:
synthesising a loop-ligated precursor polynucleotide by connecting a 3′-end of the forward strand of the target polynucleotide and a 5′-end of the reverse strand of the target polynucleotide with a loop, or connecting a 5′-end of the forward strand of the target polynucleotide and a 3′-end of the reverse strand of the target polynucleotide with a loop,
synthesising the at least one first polynucleotide sequence comprising the first portion by forming a complement of the loop-ligated precursor polynucleotide, and
synthesising the at least one second polynucleotide sequence comprising the at least one second polynucleotide sequence by forming a complement of the at least one first polynucleotide sequence.

Typically, the loop may be generated by attaching a first flanking adaptor to the target (double-stranded) polynucleotide.

The first flanking adaptor may be an oligonucleotide of any structure or any sequence that allows the forward and reverse strands to be connected via a loop. In one embodiment, the first flanking adaptor comprises a base-paired stem and a hairpin loop (e.g. a loop structure with unpaired or non-Watson-Crick paired nucleotides) and connects the 3′ end of the forward strand with the 5′ end of the reverse strand, or the 5′ end of the forward strand with the 3′ end of the reverse strand.

The step of synthesising the loop-ligated precursor polynucleotide may further comprise connecting a 5′-end of the forward strand of the target polynucleotide and a 3′-end of the reverse strand of the target polynucleotide (when the 3′-end of the forward strand of the target polynucleotide and the 5′-end of the reverse strand of the target polynucleotide are connected with a loop), or a 3′-end of the forward strand of the target polynucleotide and a 5′-end of the reverse strand of the target polynucleotide (when the 5′-end of the forward strand of the target polynucleotide and the 3′-end of the reverse strand of the target polynucleotide are connected with a loop), with a second flanking adaptor.

In one embodiment, the second flanking adaptor comprises a base-paired stem, a primer-binding sequence and a primer-binding complement sequence. Specifically, the second flanking adaptor may comprise a first and second strand, wherein the first and second strands are base-paired for a portion of their sequence (forming the base-paired stem) and are non-complementary for the remainder of their sequence, for example, P5′ and P7 or P7′ and P5, which subsequently forms a fork structure, wherein a first arm of the fork structure comprises a primer-binding sequence and the second arm of the fork structure comprises a primer-binding complement sequence. In an alternative embodiment, the second flanking adaptor may comprise a base-paired stem and a hairpin loop, where the loop comprises a primer-binding sequence, a cleavable site and primer-binding complement sequence, where the cleavable site is in-between the primer-binding sequence and the primer-binding complement sequence. In this alternative embodiment, the method may comprise cleaving the loop of the second flanking adaptor at the cleavable site to open the loop. This will generate a fork structure, as described above. Specifically, following cleavage the second flanking adaptor will form a base-paired stem and then a fork.

As used herein for the second flanking adaptor, by “cleavable site” is meant any moiety, such as a modified nucleotide, that allows selective cleavage of the second flanking adaptor sequence. By way of non-limiting example, the cleavable site may comprise uracil bases, phosphorothioate groups, ribonucleotides, diol linkages, disulphide linkages, peptides etc.

In one example, the cleavable site is a uracil. Uracil can be cleaved using a uracil glycosylase or USER enzyme mix (which is a cocktail of uracil glycosylase and endonuclease VIII). In another example, the cleavable site is 8-oxoguanine. 8-oxoguanine can be cleaved using a FPG glycosylase. Alternatively, the cleavable site is a restriction site.

By “restriction site” is meant a sequence of nucleotides recognised by an endonuclease, such as a single-stranded endonuclease. A restriction site may also be referred to as a “recognition site” or “recognition sequence”, and such terms may be used interchangeably.

In one embodiment, the endonuclease is a single strand restriction endonuclease, a nicking endonuclease or nicking enzyme or nickase (again, such terms may be used interchangeably). By any of these terms is meant an enzyme that can hydrolyze only one strand of the double-stranded polynucleotide (duplex), to produce DNA molecules that are “nicked”, rather than fully cleaved on both strands.

Examples of suitable nicking enzymes that may be used include, but are not limited to, Nb.BbvCI, Nb.Bsml, Nb.BsrDI, Nb. Btsl, Nt.Alwl, Nt. BsmAl, Nt.BspQI, Nt.BstNBI, BssSI, Nb.Bpu101 and Nt.CviPII, These nickases can be used either alone or in various combinations. Other suitable nicking endonucleases are available from commercial sources, including New England Biolabs and Fisher Scientific.

In one embodiment, the second flanking adaptor comprises at least one primer-binding sequence. In another embodiment, the second flanking adaptor comprises at least one primer-binding complement sequence. In another embodiment, the second flanking adaptor comprises both a primer-binding sequence and a primer-binding complement sequence. The primer-binding sequence may be capable of binding to a lawn or immobilised primer that is immobilised on the surface of a solid support. For example, the primer-binding sequence may be either P5′ (for example, SEQ ID NO: 3 or 6 or a variant or fragment thereof) or P7′ (for example, SEQ ID NO: 4 or a variant or fragment thereof). Similarly, the primer-binding complement sequence may be either P5 (for example, SEQ ID NO: 1 or 5 or a variant or fragment thereof) or P7 (for example, SEQ ID NO: 2 or a variant or fragment thereof). If the primer-binding sequence is P5′, the primer-binding complement sequence is P7. If the primer-binding sequence is P7′, the primer-binding complement sequence is P5.

At least one of the first flanking adaptor and the second flanking adaptor comprises a restriction site for an endonuclease, such as a single-stranded endonuclease. If the second flanking adaptor comprises a base-paired stem and a hairpin loop structure, then the restriction site for an endonuclease is additional to the cleavable site. Where the restriction site is present in the first flanking adaptor, this allows a nick to be generated in the template and/or template complement strands in the loop (and/or loop complement) formed from the first flanking adaptor. Where the restriction site is present in the second flanking adaptor, this allows a nick to be generated close to the first immobilised primer and/or the second immobilised primer. Where nicking is used, such a nick prepares the strands for sequencing, since sequencing can be initiated from the nick (e.g. using strand displacement SBS), or allows non-immobilised polynucleotide sequences to be washed away to enable binding of sequencing primers.

In one embodiment, the endonuclease is a single strand restriction endonuclease, a nicking endonuclease or nicking enzyme or nickase (again, such terms may be used interchangeably). By any of these terms is meant an enzyme that can hydrolyze only one strand of the double-stranded polynucleotide (duplex), to produce DNA molecules that are “nicked”, rather than fully cleaved on both strands.

Examples of suitable nicking enzymes that may be used include, but are not limited to, Nb. BbvCI, Nb.Bsml, Nb.BsrDI, Nb. Btsl, Nt.Alwl, Nt. BsmAl, Nt.BspQI, Nt.BstNBI, BssSI, Nb.Bpu101 and Nt.CviPII, These nickases can be used either alone or in various combinations. Other suitable nicking endonucleases are available from commercial sources, including New England Biolabs and Fisher Scientific.

The first and second flanking adaptors also may comprise one or more sequencing primer-binding sites (or sequencing primer-binding site complements). The sequencing primer-binding sites and the sequencing primer-binding site complements may allow binding of a sequencing primer.

In the first flanking adaptor the sequencing primer-binding sites may be in the loop sequence or in the base-paired stem. In one embodiment, the base-paired stem comprises at least one sequencing primer-binding site. In another embodiment, the sequencing primer-binding site is in the base-paired stem, and in the part of the stem that connects to the reverse strand of the double-stranded polynucleotide. In another embodiment, the loop may comprise two sequencing primer-binding sites. In one embodiment, the loop comprises two sequencing primer-binding sites and a restriction site, wherein the sequencing primer-binding sites are either side of the restriction site.

In the second flanking adaptor the sequencing primer-binding site(s) may also be in the base-paired stem. Alternatively, each fork of the second flanking adaptor may additionally comprise a sequencing primer-binding site.

The sequencing primer binding sites are sequencing and/or index primer binding sites and indicate the starting point of the sequencing read. During the sequencing process, a sequencing primer anneals (i.e. hybridises) to at least a portion of the sequencing primer binding site on the template strand. The polymerase enzyme binds to this site and incorporates complementary nucleotides base by base into the growing opposite strand.

The sequence of the sequencing primers and the sequence primer binding sites are not material to the methods of the invention, as long as the sequencing primers are able to bind to the sequence primer binding site (or sequencing binding site complement) to enable amplification and sequencing of the regions to be identified.

In some embodiments, the restriction site in the first flanking adaptor is in the middle of the loop or substantially the middle of the loop. In particular, the restriction site may be cleavable by a double strand restriction endonuclease or restriction enzyme. By either of these terms is meant an enzyme that can hydrolyze both strands of the double-stranded polynucleotide (duplex), to produce polynucleotide molecules that are cleaved on both strands. In one embodiment, the restriction enzyme is a type II restriction enzyme.

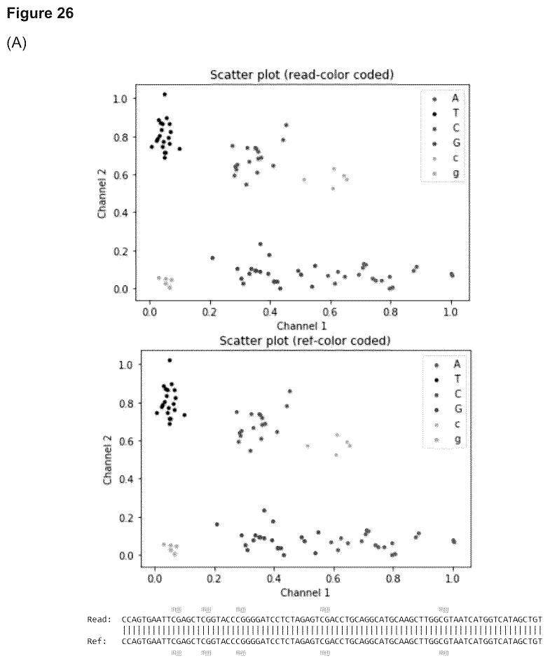

FIGS. 21 to 23 illustrate various ways in which first portions and second portions can be prepared for concurrent sequencing.

FIG. 21 shows how concurrent sequencing is enabled by nicking after a 3′-end of the first portion, and nicking after a 3′-end of the second portion. Here, the nicks are made at a 3′-end of both the loop and loop complement. In one case, non-immobilised strands may be washed away and standard SBS can be conducted, resulting in concurrent sequencing of the first and second portions. In an alternative case, the non-immobilised strands are not washed away and SBS can be conducted using a strand displacement polymerase, again resulting in concurrent sequencing of the first and second portions.

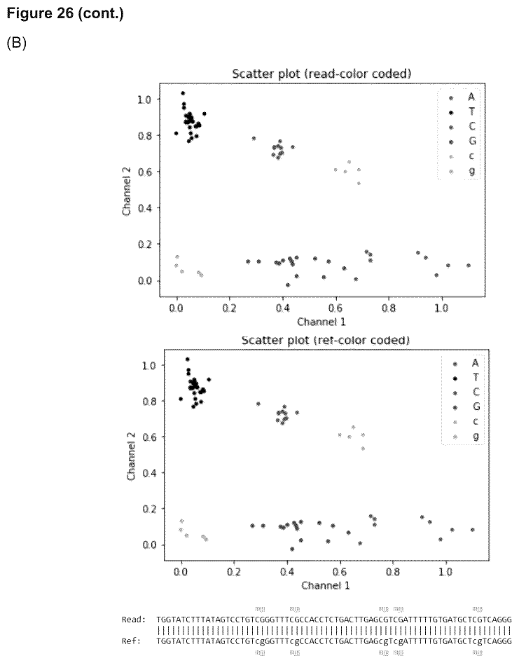

FIG. 22 shows how concurrent sequencing is enabled by nicking before a 5′-end of the first portion, and nicking before a 5′-end of the second portion. Here, the nicks are made after a 3′-end of the first immobilised primer and after a 3′-end of the second immobilised primer. SBS can then be conducted using a strand displacement polymerase, resulting in concurrent sequencing of the first and second portions.

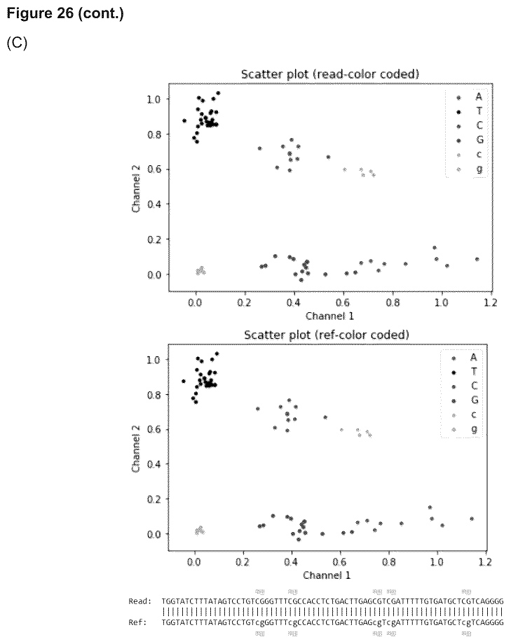

FIG. 23 shows how concurrent sequencing is enabled by contacting first sequencing primer binding sites located after a 3′-end of the first portions with first primers and second sequencing primer binding sites located after a 3′-end of the second portions with second primers. Here, a middle portion of the loop and loop complement may be cleaved (e.g. with a double strand restriction endonuclease or restriction enzyme). The non-immobilised strands may be washed away, and any remaining sections of the loop and loop complement can act as sequencing primer binding sites, allowing standard SBS to be conducted resulting in concurrent sequencing of the first and second portions.

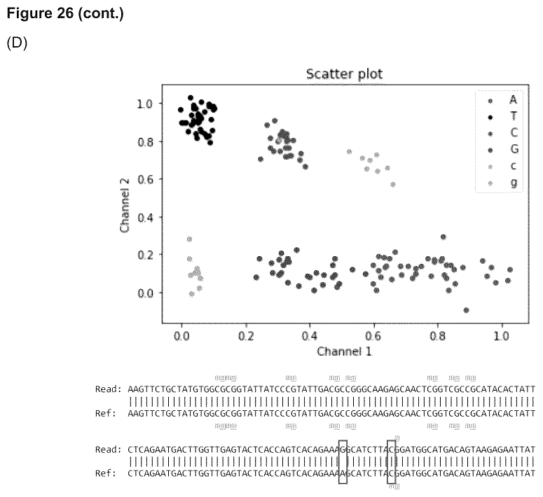

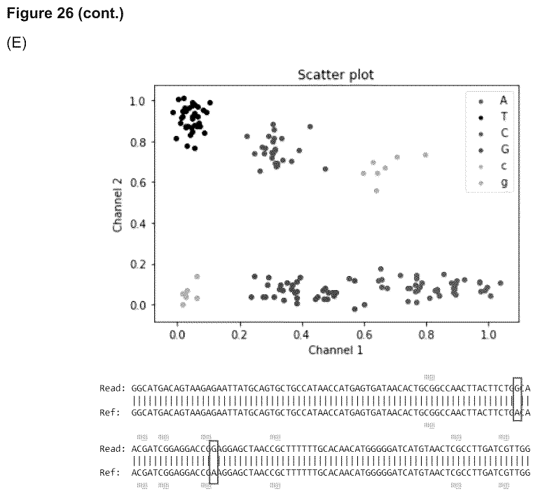

It is also possible to conduct paired end reads using these methods. FIGS. 24 and 25 illustrate various ways in which paired end reads can be achieved.

FIG. 24 shows paired end reads being conducted after a first round of concurrent sequencing as shown in FIG. 21. Further nicks can be made after a 3′-end of the first immobilised primer and after a 3′-end of the second immobilised primer. SBS can then be conducted using a strand displacement polymerase, resulting in concurrent sequencing of complements of the first and second portions.

FIG. 25 shows paired end reads being conducted after a first round of concurrent sequencing as shown in FIG. 22. Any free 3′-ends can be blocked. Further nicks can be made after a 3′-end of the first portion, and after a 3′-end of the second portion, then SBS can then be conducted using a strand displacement polymerase, resulting in concurrent sequencing of complements of the first and second portions.

Although not shown in FIG. 23, paired end reads can also be conducted after Read 1.1 and Read 1.2. This can be achieved by having further immobilised primers on the solid support that are substantially complementary to the remaining sections of the loop and loop complement acting as sequencing primer binding sites. This allows resynthesis of the strands, and subsequent binding of further sequencing primers for concurrent sequencing of complements of the first and second portions.

In some embodiments, the method may further comprise a step of concurrently sequencing nucleobases in the first portion and the second portion.

The target polynucleotide (or in some embodiments, the polynucleotide library) has been pre-treated using a conversion reagent. In some embodiments, the method of preparing at least one polynucleotide sequence for detection of modified cytosines may include a step of treating the target polynucleotide using a conversion agent.

The conversion reagent is configured to convert a modified cytosine to thymine or a nucleobase which is read as thymine/uracil, and/or is configured to convert an unmodified cytosine to uracil or a nucleobase which is read as thymine/uracil.

As used herein, the term “modified cytosine” may refer to any one or more of 5-methylcytosine (5-mC), 5-hydroxymethylcytosine (5-hmC), 5-formylcytosine (5-fC) and 5-carboxylcytosine (5-caC):

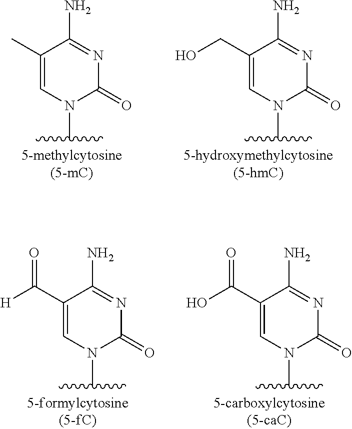

wherein the wavy line indicates an attachment point of the modified cytosine to the polynucleotide.

As used herein, the term “unmodified cytosine” refers to cytosine (C):

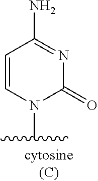 [SMILES: *n1ccc(N)nc1=O]

wherein the wavy line indicates an attachment point of the unmodified cytosine to the polynucleotide.

As used herein, the term “conversion reagent configured to convert a modified cytosine to thymine or a nucleobase which is read as thymine/uracil” may refer to a reagent which converts one more or modified cytosines (e.g. 5-methylcytosine, 5-hydroxymethylcytosine, 5-formylcytosine and 5-carboxylcytosine) to thymine (i.e. would base pair with adenine), or to an equivalent nucleobase which would base pair with adenine. The conversion may comprise a deamination reaction converting the modified cytosine to thymine or nucleobase which is read as thymine/uracil.

As used herein, the term “conversion reagent configured to convert an unmodified cytosine to uracil or a nucleobase which is read as thymine/uracil” may refer to a reagent which converts one or more unmodified cytosines to uracil (i.e. would base pair with adenine), or to an equivalent nucleobase which would base pair with adenine. The conversion may comprise a deamination reaction converting the unmodified cytosine to uracil or nucleobase which is read as thymine/uracil.

In general, if modified cytosines were present in the target polynucleotide to be sequenced, the forward strand of the template will then not be identical to the reverse complement strand of the template as a result of treatment of the target polynucleotide with the conversion agent (alternatively, the reverse strand of the template will then not be identical to the forward complement strand of the template as a result of treatment of the target polynucleotide with the conversion agent). However, if modified cytosines were not present in the target polynucleotide to be sequenced, the forward strand of the template will then be (substantially) identical to the reverse complement strand of the template despite treatment of the target polynucleotide with the conversion agent (alternatively, the reverse strand of the template will then be (substantially) identical to the forward complement strand of the template despite treatment of the target polynucleotide with the conversion agent). As such, mismatches between the forward strand of the template and the reverse complement strand of the template allow the detection of modified cytosines (alternatively, mismatches between the reverse strand of the template and the forward complement strand of the template allow detection of modified cytosines).

Where the forward strand (or reverse strand) of the template is not identical to the reverse complement strand (or forward complement strand) of the template as a result of treatment with the conversion agent, the forward strand (or reverse strand) of the template may comprise a guanine base at a first position, which leads to a basecall of C for the original target polynucleotide; and wherein the reverse complement strand (or forward complement strand) of the template may comprise an adenine base at a second position corresponding to the same position number as the first position, which leads to a basecall of T for the original target polynucleotide. The adenine base at the second position within the template may have been generated as a result of conversion of modified cytosines in the target polynucleotide to thymine, or to an equivalent nucleobase which would base pair with adenine; or may have been generated as a result of conversion of unmodified cytosines in the target polynucleotide to uracil, or to an equivalent nucleobase which would base pair with adenine. In particular, the adenine base at the second position within the template may have been generated as a result of conversion of unmodified cytosines in the target polynucleotide to uracil, or to an equivalent nucleobase which would base pair with adenine.

In other cases, the forward strand (or reverse strand) of the template comprises an adenine base at a first position, which leads to a basecall of T for the original target polynucleotide; and wherein the reverse complement strand (or forward complement strand) of the template comprises a guanine base at a second position corresponding to the same position number as the first position, which leads to a basecall of C for the original target polynucleotide. Similarly, the adenine base at the first position within the template may have been generated as a result of conversion of modified cytosines in the target polynucleotide to thymine, or to an equivalent nucleobase which would base pair with adenine; or may have been generated as a result of conversion of unmodified cytosines in the target polynucleotide to uracil, or to an equivalent nucleobase which would base pair with adenine. In particular, the adenine base at the first position within the template may have been generated as a result of conversion of modified cytosines in the target polynucleotide to thymine, or to an equivalent nucleobase which would base pair with adenine.

In some embodiments, the conversion reagent configured to convert a modified cytosine to thymine or a nucleobase which is read as thymine/uracil may further be configured to be selective for converting one or more modified cytosines (e.g. 5-methylcytosine, 5-hydroxymethylcytosine, 5-formylcytosine and 5-carboxylcytosine) over converting unmodified cytosine. The selectivity may be measured by comparing reaction parameters (e.g. deamination reaction parameters) of the conversion of a particular modified cytosine to thymine or equivalent nucleobase which is read as thymine/uracil, with corresponding reaction parameters (e.g. deamination reaction parameters) of the conversion of unmodified cytosine to uracil or nucleobase which is read as thymine/uracil. For example, reaction parameters such as rate of reaction or yield may be compared. In the case of rate of reaction, a rate of a reaction (e.g. deamination) of the particular modified cytosine to thymine or nucleobase which is read as thymine/uracil may be greater (e.g. at least 2 times greater, at least 5 times greater, at least 10 times greater, at least 20 times greater, at least 50 times greater, or at least 100 times greater) than a corresponding rate of a reaction (e.g. deamination) of the unmodified cytosine to uracil or nucleobase which is read as thymine/uracil. In the case of yield, a yield of a reaction (e.g. deamination) of the particular modified cytosine to thymine or nucleobase which is read as thymine/uracil may be greater (e.g. at least 2 times greater, at least 5 times greater, at least 10 times greater, at least 20 times greater, at least 50 times greater, or at least 100 times greater) than a corresponding yield of a reaction (e.g. deamination) of the unmodified cytosine to uracil or nucleobase which is read as thymine/uracil.

In some embodiments, the conversion reagent configured to convert an unmodified cytosine to uracil or a nucleobase which is read as thymine/uracil may further be configured to be selective for converting unmodified cytosine over converting one or more modified cytosines (e.g. 5-methylcytosine, 5-hydroxymethylcytosine, 5-formylcytosine and 5-carboxylcytosine). The selectivity may be measured by comparing reaction parameters (e.g. deamination reaction parameters) of the conversion of unmodified cytosine to uracil or nucleobase which is read as thymine/uracil, with corresponding reaction parameters (e.g. deamination reaction parameters) of the conversion of a particular modified cytosine to thymine or nucleobase which is read as thymine/uracil. For example, reaction parameters such as rate of reaction or yield may be compared. In the case of rate of reaction, a rate of a reaction (e.g. deamination) of the unmodified cytosine to uracil or nucleobase which is read as thymine/uracil may be greater (e.g. at least 2 times greater, at least 5 times greater, at least 10 times greater, at least 20 times greater, at least 50 times greater, or at least 100 times greater) than a rate of a reaction (e.g. deamination) of the particular modified cytosine to uracil or the nucleobase which is read as thymine/uracil. In the case of yield, a yield of a reaction (e.g. deamination) the unmodified cytosine to uracil or nucleobase which is read as thymine/uracil may be greater (e.g. at least 2 times greater, at least 5 times greater, at least 10 times greater, at least 20 times greater, at least 50 times greater, or at least 100 times greater) than a corresponding yield of a reaction (e.g. deamination) of the particular modified cytosine to uracil or the nucleobase which is read as thymine/uracil.

In some embodiments, the conversion agent may comprise a chemical agent and/or an enzyme.

In some embodiments, the chemical agent may comprise a boron-based reducing agent. In one embodiment, the boron-based reducing agent is an amine-borane compound or an azine-borane compound (wherein the term “azine” refers to a nitrogenous heterocyclic compound comprising a 6-membered aromatic ring). Non-limiting examples of amine-borane compounds include compounds such as t-butylamine borane, ammonia borane, ethylenediamine borane and dimethylamine borane. Non-limiting examples of azine-borane compounds include compounds such as pyridine borane and 2-picoline borane.

In general, boron-based reducing agents are able to convert 5-formylcytosine and 5-carboxylcytosine to dihydrouracil (i.e. a nucleobase which is read as thymine/uracil). The reaction proceeds by reduction of the internal C═C bond of 5-formylcytosine or 5-carboxylcytosine, deamination, and then decarboxylation to form dihydrouracil (illustrated below using 5-carboxylcytosine):

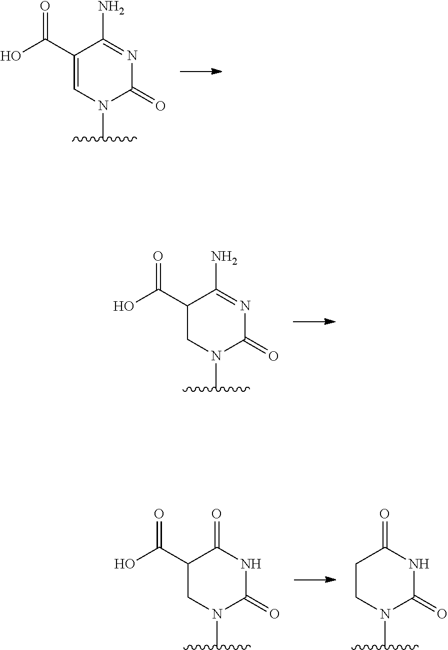 [SMILES: *n1cc(C(=O)O)c(N)nc1=O ; *N1CC(C(=O)O)C(N)=NC1=O ; *N1CC(C(=O)O)C(=O)NC1=O ; *N1CCC(=O)NC1=O]

This process is selective for a particular type of modified cytosine (5-carboxylcytosine) and does not convert unmodified cytosine. Where distinction between other modified cytosines and unmodified cytosines is desired (or even between different types of modified cytosines), treatment with further agents as described herein prior to treatment with the boron-based reducing agent may provide such distinction. In particular, boron-based reducing agents may be combined with ten-eleven translocation (TET) methylcytosine dioxygenases, B-glucosyltransferases, oxidising agents, oximes and/or hydrazones as described herein.

In some embodiments, the chemical agent may comprise sulfite. The sulfite may be present in a partially acid/salt form (e.g. as bisulfite ions), or be present in a salt form (e.g. as sulfite ions). In cases where the sulfite is present in a salt form, the sulfite may comprise a cation (not including H⁺). For example, the cation may be selected from “metal cations” or “non-metal cations”. Metal cations may include alkali metal ions (e.g. lithium, sodium, potassium, rubidium or caesium ions). Non-metal cations may include ammonium salts (e.g. alkylammonium salts) or phosphonium salts (e.g. alkylphosphonium salts). The term “sulfite” also encompasses “metabisulfite”, which dissolves in aqueous solution to form bisulfite.

In general, sulfite (e.g. bisulfite) is able to convert unmodified cytosine to uracil. The reaction proceeds via conjugate addition of sulfite to the internal C═C of unmodified cytosine, deamination, and then elimination of sulfite to reform the internal C═C bond to form uracil:

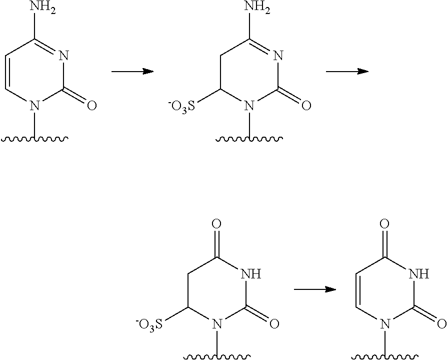

This process is selective for unmodified cytosine over certain types of modified cytosine (5-methylcytosine and 5-hydroxymethylcytosine). However, 5-formylcytosine and 5-carboxylcytosine are converted to their equivalent deaminated versions. Where distinction between different types of modified cytosines (e.g. 5-formylcytosine and 5-carboxylcytosine) is desired, treatment with further agents as described herein prior to treatment with the sulfite may provide such distinction. In particular, the sulfite may be combined with ten-eleven translocation (TET) methylcytosine dioxygenases, β-glucosyltransferases, oxidising agents and/or reducing agents as described herein.

In one embodiment, the enzyme may comprise a cytidine deaminase.

As used herein, the term “cytidine deaminase” may refer to an enzyme which is able to catalyse the following reaction:

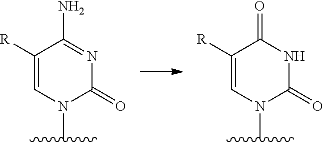

wherein R is hydrogen, methyl, hydroxymethyl, formyl or carboxyl, and wherein the wavy line indicates an attachment point to a polynucleotide.

In one embodiment, the cytidine deaminase is a wild-type cytidine deaminase or a mutant cytidine deaminase. In one example, the cytidine deaminase is a mutant cytidine deaminase.

In some embodiments, the cytidine deaminase is a member of the APOBEC protein family. In one embodiment, the cytidine deaminase is a member of the AID subfamily, the APOBEC1 subfamily, the APOBEC2 subfamily, the APOBEC3 subfamily (e.g. the APOBEC3A subfamily, the APOBEC3B subfamily, the APOBEC3C subfamily, the APOBEC3D subfamily, the APOBEC3F subfamily, the APOBEC3G subfamily, or the APOBEC3H subfamily), or the APOBEC4 subfamily. In one example, the cytidine deaminase is a member of the APOBEC3A subfamily.

In general, cytidine deaminases are able to catalyse the deamination of all modified cytosines (particularly 5-methylcytosine, 5-hydroxymethylcytosine and 5-formylcytosine) to their equivalent deaminated versions (i.e. nucleobases which are read as thymine/uracil), as well as catalysing the deamination of unmodified cytosines to uracil. Nevertheless, rates of reaction may differ depending on the type of modified cytosine; for example, wild-type APOBEC3A catalyses the deamination of unmodified cytosine and 5-methylcytosine relatively efficiently, whereas deamination of 5-hydroxymethylcytosine is ˜5000-fold slower relative to unmodified cytosine, deamination of 5-formylcytosine is ˜3700-fold slower relative to unmodified cytosine, and deamination of 5-carboxylcytosine is >20000-fold slower relative to unmodified cytosine. Where distinction between modified cytosines and unmodified cytosines is desired (or even between different types of modified cytosines), treatment with further agents as described herein prior to treatment with the cytidine deaminase may provide such distinction. In particular, the cytidine deaminase may be combined with ten-eleven translocation (TET) methylcytosine dioxygenases and/or β-glucosyltransferases as described herein. Alternatively, or in addition, particular cytidine deaminases (e.g. mutant cytidine deaminases) may be chosen which have higher affinities for modified cytosines as substrates over unmodified cytosines, or vice versa.

The APOBEC protein family is a member of the large cytidine deaminase superfamily that contains a canonical zinc-dependent deaminase (ZDD) signature motif embedded within a core cytidine deaminase fold. This fold includes a five-stranded mixed beta (b)-sheet surrounded by six alpha (a)-helices with the order a1-b1-b2-a2-b3-a3-b4-a4-b5-a5-a6 (Salter et al., Biochem Trends Sci. 2016 41(7):578-594. doi:10.1016/j.tibs.2016.05.001; Salter et al., Trends Biochem. Sci. 2018, 43(8):606-622 doi.org/10.1016/j.tibs.2018.04.013). Each cytidine deaminase domain core structure of APOBEC proteins contains a highly conserved spatial arrangement of the catalytic centre residues of a zinc-binding motif H—[P/A/V]-E-X_{[23-28]}—P—C—X_{[2-4]}—C(SEQ ID NO: 51) (referred to herein as the ZDD motif, where X is any amino acid, and the subscript range of numbers after X refers to the number of amino acids) (Salter et al., Trends Biochem Sci. 2016 41(7):578-594. doi:10.1016/j.tibs.2016.05.001). Without intending to be limited by theory, the H and two C residues coordinate a Zn atom, and the E residue polarises a water molecule near the Zn-atom for catalysis (Chen et al., 2021, Viruses, 13:497, doi.org/10.3390/v13030497).

Some members of the APOBEC protein family, e.g., the AID subfamily, the APOBEC1 subfamily, the APOBEC2 subfamily, the APOBEC3A subfamily, the APOBEC3C subfamily, the APOBEC3H subfamily, and the APOBEC4 subfamily, include one copy of the ZDD motif. Other members of the APOBEC protein family, e.g., the APOBEC3B subfamily, the APOBEC3D subfamily, the APOBEC3F subfamily, and the APOBEC3G subfamily, include two copies of the ZDD motif, but often only the C-terminal copy is active (Salter et al., Trends Biochem Sci. 2016 41(7):578-594. doi:10.1016/j.tibs.2016.05.001). Thus, a mutant cytidine deaminase disclosed herein includes one or two ZDD motifs. In one embodiment, a mutant cytidine deaminase based on a member of the APOBEC3A subfamily includes the following ZDD motif: HXEX₂₄SW(S/T)PCX_{[2-4]}CX₆FX₈LX₅R(L/I)YX_{[8-11]}LX₂LX_{[10]}M (SEQ ID NO: 52) (where X is any amino acid, and the subscript number or range of numbers after X refers to the number of amino acids) (Salter et al., Trends Biochem Sci. 2016 41(7):578-594. doi:10.1016/j.tibs.2016.05.001).

Non-limiting examples of wild-type cytidine deaminases in the APOBEC protein family are shown in the table below (from UniProt, database of protein sequence and functional information, available at uniprot.org; or GenBank, collection of nucleotide sequences and their protein translations, available at ncbi.nlm.nih.gov/protein/):

| APOBEC protein | Non-limiting examples |
|---|---|
| AID | UniProt: Q9GZX7 (SEQ ID NO: 7); |
| | UniProt: G3QLD2 (SEQ ID NO: 8); |
| | Uniprot Q9WVE0 (SEQ ID NO: 9) |
| APOBEC1 | UniProt: P41238 (SEQ ID NO: 10); |
| | NCBI XP_030856728.1 (SEQ ID NO: 11); |
| | Uniprot P51908 (SEQ ID NO: 12) |
| APOBEC2 | UniProt: Q9Y235 (SEQ ID NO: 13); |
| | Uniprot G3SGN8 (SEQ ID NO: 14); |
| | Uniprot Q9WV35 (SEQ ID NO: 15) |
| APOBEC3A | UniProt: P31941(SEQ ID NO: 16); |
| | GenBank: XP_045219544.1 (SEQ ID NO: 17) |
| | GenBank: AER45717.1 (SEQ ID NO: 18); |
| | GenBank: XP_003264816.1 (SEQ ID NO: 19); |
| | GenBank: PNI48846.1 (SEQ ID NO: 20); |
| | GenBank: ADO85886.1 (SEQ ID NO: 21) |
| APOBEC3B | UniProt: Q9UH17 (SEQ ID NO: 22); |
| | Uniprot G3QV16 (SEQ ID NO: 23); |
| | Uniprot F6M3K5 (SEQ ID NO: 24) |
| APOBEC3C | UniProt: Q9NRW3 (SEQ ID NO: 25); |
| | Uniprot Q694B5 (SEQ ID NO: 26); |
| | Uniprot B0LW74 (SEQ ID NO: 27) |
| APOBEC3D | UniProt: Q96AK3 (SEQ ID NO: 28); |
| | NCBI NP_001332895.1 (SEQ ID NO: 29); |
| | NCBI NP_001332931.1 (SEQ ID NO: 30) |
| APOBEC3F | UniProt: Q8IUX4 (SEQ ID NO: 31); |
| | Uniprot G3RD21 (SEQ ID NO: 32); |
| | Uniprot Q1G0Z6 (SEQ ID NO: 33) |
| APOBEC3G | UniProt: Q9HC16 (SEQ ID NO: 34); |
| | Uniprot Q694C1 (SEQ ID NO: 35); |
| | Uniprot U5NDB3 (SEQ ID NO: 36) |
| APOBEC3H | UniProt: Q6NTF7 (SEQ ID NO: 37); |
| | Uniprot B7T0U7 (SEQ ID NO: 38); |
| | Uniprot Q19Q52 (SEQ ID NO: 39) |
| APOBEC4 | UniProt: Q8WW27(SEQ ID NO: 40); |
| | NCBI XP_004028087.1 (SEQ ID NO: 41); |
| | Uniprot Q497M3 (SEQ ID NO: 42) |

In one embodiment, the mutant cytidine deaminase may comprise amino acid substitution mutations at positions functionally equivalent to (Tyr/Phe)130 and Tyr132 in a wild-type APOBEC3A protein. Such mutant cytidine deaminases are described in further detail in U.S. Provisional Application No. 63/328,444, which is incorporated herein by reference. By “functionally equivalent” it is meant that the mutant cytidine deaminase has the amino acid substitution at the amino acid position in a reference (wild-type) cytidine deaminase that has the same functional role in both the reference (wild-type) cytidine deaminase and the mutant cytidine deaminase.

In one embodiment, the (Tyr/Phe) 130 may be Tyr130, and the wild-type APOBEC3A protein may be SEQ ID NO: 16.

In some embodiments, the mutant cytidine deaminase may convert 5-methylcytosine to thymine by deamination at a greater rate than conversion rate of cytosine to uracil by deamination; wherein the rate may be at least 100-fold greater.

In one embodiment, the substitution mutation at the position functionally equivalent to Tyr130 may comprise Ala, Val or Trp.

In one embodiment, the substitution mutation at the position functionally equivalent to Tyr132 may comprise a mutation to His, Arg, Gln or Lys.

In one embodiment, the mutant cytidine deaminase may comprise a ZDD motif H—[P/A/V]-E-X_{[23-28]}—P—C—X_{[2-4]}—C(SEQ ID NO: 51).

In one embodiment, the mutant cytidine deaminase may be a member of the APOBEC3A subfamily and may comprise a ZDD motif HXEX₂₄SW(S/T)PCX_{[2-4]}CX₆FX₈LX₅R(L/I)YX_{[8-11]}LX₂LX_{[10]}M (SEQ ID NO: 52).

In some embodiments, the target polynucleotide may be treated with a further agent prior to treatment with the conversion reagent.

In one embodiment, the further agent may be configured to convert a modified cytosine (e.g. one of 5-methylcytosine, 5-hydroxymethylcytosine, 5-formylcytosine and 5-carboxylcytosine) to another modified cytosine (e.g. another one of 5-methylcytosine, 5-hydroxymethylcytosine, 5-formylcytosine and 5-carboxylcytosine).

For example, the further agent may be configured to convert 5-methylcytosine to 5-hydroxymethylcytosine. In the same or other embodiments, the further agent may be configured to convert 5-hydroxymethylcytosine to 5-formylcytosine. In the same or other embodiments, the further agent may be configured to convert 5-formylcytosine to 5-carboxylcytosine. In some embodiments, the further agent may be configured to convert 5-methylcytosine to 5-hydroxymethylcytosine, 5-hydroxymethylcytosine to 5-formylcytosine, and 5-formylcytosine to 5-carboxylcytosine.

In other embodiments, the further agent may be configured to convert 5-formylcytosine to 5-hydroxymethylcytosine.

In another embodiment, the further agent configured to convert a modified cytosine (e.g. one of of 5-methylcytosine, 5-hydroxymethylcytosine, 5-formylcytosine and 5-carboxylcytosine) to another modified cytosine (e.g. another (different) one of 5-methylcytosine, 5-hydroxymethylcytosine, 5-formylcytosine and 5-carboxylcytosine) may comprise a chemical agent and/or an enzyme.

The further agent configured to convert a modified cytosine to another modified cytosine may be a chemical agent; for example, an oxidising agent; such as a metal-based oxidising agent; for example, a transition metal-based oxidising agent; such as a ruthenium-based oxidising agent. The oxidising agent may be configured to convert 5-hydroxymethylcytosine to 5-formylcytosine. Non-limiting examples of the oxidising agent include ruthenate (e.g. potassium ruthenate, K₂RuO₄), or perruthenate (e.g. potassium perruthenate, KRuO₄).

The further agent configured to convert a modified cytosine to another modified cytosine may be a chemical agent; such as a reducing agent; for example, a Group III-based reducing agent; such as a boron-based reducing agent. The oxidising agent may be configured to convert 5-formylcytosine to 5-hydroxymethylcytosine. Non-limiting examples of the reducing agent include borohydride (e.g. sodium borohydride, lithium borohydride), or triacetoxyborohydride (e.g. sodium triacetoxyborohydride).

The further agent configured to convert a modified cytosine to another modified cytosine may be an enzyme; such as a ten-eleven translocation (TET) methylcytosine dioxygenase; wherein the TET methylcytosine dioxygenase may be a member of the TET1 subfamily, the TET2 subfamily, or the TET3 subfamily. The enzyme may be configured to convert 5-methylcytosine to 5-hydroxymethylcytosine, 5-hydroxymethylcytosine to 5-formylcytosine, and 5-formylcytosine to 5-carboxylcytosine. Non-limiting examples of the TET methylcytosine dioxygenase include:

| TET protein | Non-limiting examples |
|---|---|
| TET1 | UniProt: Q8NFU7 (SEQ ID NO: 43) |
| | UniProt: Q3URK3 (SEQ ID NO: 44) |
| TET2 | UniProt: Q6N021 (SEQ ID NO: 45) |
| | UniProt: Q4JK59 (SEQ ID NO: 46) |
| TET3 | UniProt: O43151 (SEQ ID NO: 47) |
| | UniProt: Q8BG87 (SEQ ID NO: 48) |

In one embodiment, the further agent may be configured to reduce/prevent deamination of a particular modified cytosine (e.g. one of 5-methylcytosine, 5-hydroxymethylcytosine, 5-formylcytosine and 5-carboxylcytosine). Such a further agent configured to reduce/prevent deamination of a particular modified cytosine may be used in combination with a further agent configured to convert a modified cytosine to another modified cytosine.

For example, the further agent may be configured to convert 5-hydroxymethylcytosine to a 5-hydroxymethylcytosine analogue bearing a hydroxyl protecting group. The 5-hydroxymethylcytosine analogue bearing the hydroxyl protecting group may be resistant to oxidation to form 5-formylcytosine. Non-limiting examples of hydroxyl protecting groups include sugar groups (e.g. glycosyl), silyl ether groups (e.g. trimethylsilyl, triethylsilyl, triisopropylsilyl, t-butyl(dimethyl)silyl, t-butyl(diphenyl)silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl).

In other embodiments, the further agent may be configured to convert 5-formylcytosine to a 5-formylcytosine analogue bearing an oxime or a hydrazone group. The 5-formylcytosine analogue bearing the oxime or hydrazone group may be resistant to oxidation to form 5-carboxylcytosine.

In one embodiment, the further agent configured to reduce/prevent deamination of a particular modified cytosine (e.g. one of 5-methylcytosine, 5-hydroxymethylcytosine, 5-formylcytosine and 5-carboxylcytosine) may comprise a chemical agent and/or an enzyme.

The further agent configured to reduce/prevent deamination of a particular modified cytosine may be an enzyme; such as a glycosyltransferase (e.g. α-glucosyltransferase or β-glucosyltransferase); for example, a β-glucosyltransferase. Such a further agent may be configured to convert 5-hydroxymethylcytosine to a 5-hydroxymethylcytosine analogue bearing a hydroxyl protecting group, wherein the hydroxyl protecting group is glycosyl. A non-limiting example of the enzyme includes T4-βGT, for example as supplied by New England BioLabs (catalog #M0357S, M0357L) or by ThermoFisher Scientific (catalog #EO0831); further non-limiting examples of glycosyltransferases include:

| Glucosyltransferase | Non-limiting examples |
|---|---|
| α-glucosyltransferase | UniProt: P04519 (SEQ ID NO: 49) |
| β-glucosyltransferase | UniProt: P04547 (SEQ ID NO: 50) |

The further agent configured to reduce/prevent deamination of a particular modified cytosine may be a chemical agent; such as a hydroxylamine or a hydrazine. Such a further agent may be configured to convert 5-formylcytosine to a 5-formylcytosine analogue bearing an oxime or a hydrazone group. Non-limiting examples of hydroxylamines include O-alkylhydroxylamines (e.g. O-methylhydroxylamine, O-ethylhydroxylamine), O-arylhydroxylamines (e.g. O-phenylhydroxylamine). Non-limiting examples of hydrazines include acylhydrazides (e.g. acethydrazide, benzhydrazide), alkylsulfonylhydrazides (e.g. methylsulfonylhydrazide), or arylsulfonylhydrazides (e.g. benzenesulfonylhydrazide, p-toluenesulfonylhydrazide).

Specific methods of modified cytosine sequencing using conversion agents (optionally combined with further agents) are further illustrated below. However, the type of conversion agents and/or further agents are not limited thereto.

### BS-Seq

Bisulfite sequencing (BS-seq) involves using bisulfite as the conversion agent. This process is described in Frommer et al. (Proc. Natl. Acad. Sci. U.S.A., 1992, 89, pp. 1827-1831), which is incorporated herein by reference. This process converts unmodified cytosines in the target polynucleotide to uracil, as well as 5-formylcytosine and 5-carboxylcytosine to deaminated analogues, but does not convert 5-methylcytosine and 5-hydroxymethylcytosine. Accordingly, BS-seq allows identification of the modified cytosines 5-mC and 5-hmC by reading them as C; whereas unmodified C, 5-fC and 5-caC are converted to nucleobases which are read as T/U.

### OxBS-Seq

Oxidative bisulfite sequencing (oxBS-seq) involves using potassium perruthenate as the further agent and bisulfite as the conversion agent. This process is described in Booth et al. (Science, 2012, 336, pp. 934-937), which is incorporated herein by reference. Potassium perruthenate causes oxidation of 5-hydroxymethylcytosine in the target polynucleotide to 5-formylcytosine. Subsequent treatment with bisulfite converts unmodified cytosines in the target polynucleotide to uracil, as well as 5-formylcytosine (including residues that used to be 5-hydroxymethylcytosine) and 5-carboxylcytosine to deaminated analogues, but does not convert 5-methylcytosine. Accordingly, oxBS-seq allows identification of the modified cytosine 5-mC by reading them as C; whereas unmodified C, 5-hmC, 5-fC and 5-caC are converted to nucleobases which are read as T/U.

### RedBS-Seq

Reduced bisulfite sequencing (redBS-seq) involves using sodium borohydride as the further agent and bisulfite as the conversion agent. This process is described in Booth et al. (Nat. Chem., 2014, 6, pp. 435-440), which is incorporated herein by reference. Sodium borohydride causes reduction of 5-formylcytosine in the target polynucleotide to 5-hydroxymethylcytosine. Subsequent treatment with bisulfite converts unmodified cytosines in the target polynucleotide to uracil, as well as 5-carboxylcytosine to its deaminated analogue, but does not convert 5-hydroxymethylcytosine (including residues that used to be 5-formylcytosine) and 5-methylcytosine. Accordingly, redBS-seq allows identification of the modified cytosines 5-mC, 5-hmC and 5-fC by reading them as C; whereas unmodified C and 5-caC are converted to nucleobases which are read as T/U.

### TAB-Seq

TET-assisted bisulfite sequencing (TAB-seq) involves using a T4 bacteriophage β-glucosyltransferase and a TET1 enzyme as the further agents and bisulfite as the conversion agent. This process is described in Yu et al. (Cell, 2012, 149, pp. 1368-1380), which is incorporated herein by reference. The T4 bacteriophage B-glucosyltransferase converts 5-hydroxymethylcytosine in the target polynucleotide to β-glucosyl-5-hydroxymethylcytosine, which prevents oxidation. TET1 enzyme causes oxidation of 5-methylcytosine and 5-formylcytosine in the target polynucleotide to 5-carboxylcytosine. Subsequent treatment with bisulfite converts unmodified cytosines in the target polynucleotide to uracil, as well as 5-carboxylcytosine (including residues that used to be 5-methylcytosine and 5-formylcytosine) to its deaminated analogue, but does not convert-glucosyl-5-hydroxymethylcytosine. Accordingly, TAB-seq allows identification of the modified cytosine 5-hmC (as the protected glycosyl residue) by reading it as C; whereas unmodified C, 5-mC, 5-fC and 5-caC are converted to nucleobases which are read as T/U.

### ACE-Seq

APOBEC-coupled epigenetic sequencing (ACE-seq) involves using a T4 bacteriophage B-glucosyltransferase as a further agent and APOBEC3A as the conversion agent. This process is described in Schutsky et al. (Nat. Biotechnol., 2018, 36, pp. 1083-1090), which is incorporated herein by reference. The T4 bacteriophage β-glucosyltransferase converts 5-hydroxymethylcytosine in the target polynucleotide to β-glucosyl-5-hydroxymethylcytosine, which prevents oxidation. Subsequent treatment with APOBEC3A converts unmodified cytosines in the target polynucleotide to uracil, as well as 5-methylcytosine to its deaminated analogue. 5-formylcytosine is also able to convert to its deaminated analogue, but reacts slower relative to unmodified cytosine and 5-methylcytosine. 5-carboxylcytosine is also able to convert to its deaminated analogue, but reacts far slower than unmodified cytosine and 5-methylcytosine, and slower than 5-formylcytosine. Accordingly, ACE-seq allows identification of the modified cytosine 5-hmC (as the protected glycosyl residue) by reading it as C; whereas unmodified C and 5-mC are converted to nucleobases which are read as T/U; 5-fC is converted to a nucleobase which is read as T/U to a limited extent; 5-caC is converted to a nucleobase which is read as T/U to a more limited extent.

### EM-Seq

Enzymatic Methyl sequencing (EM-seq) involves using T4 bacteriophage β-glucosyltransferase and a TET2 enzyme as the further agents and APOBEC3A as the conversion agent. This process is described in Vaisvila et al. (Genome Res. 2021, 31, pp. 1280-1289), U.S. Pat. No. 10,619,200 B2 and U.S. Pat. No. 9,121,061 B2, which are incorporated herein by reference. The T4 bacteriophage β-glucosyltransferase converts 5-hydroxymethylcytosine in the target polynucleotide to β-glucosyl-5-hydroxymethylcytosine, which prevents oxidation. The TET2 enzyme causes oxidation of 5-methylcytosine in the target polynucleotide to 5-hydroxymethylcytosine, which in turn is converted to β-glucosyl-5-hydroxymethylcytosine by the T4 bacteriophage β-glucosyltransferase. The TET2 enzyme also causes oxidation of 5-formylcytosine in the target polynucleotide to 5-carboxylcytosine. Subsequent treatment with APOBEC3A converts unmodified cytosines in the target polynucleotide to uracil, as well as 5-carboxylcytosine (including residues that used to be 5-formylcytosine) to a limited extent. Accordingly, EM-seq allows identification of the modified cytosines 5-mC and 5-hmC (as protected glycosyl residues) by reading them as C; whereas unmodified C is converted to U; 5fC and 5-caC are converted to nucleobases which are read as T/U to a limited extent.

### Modified APOBEC

Modified APOBEC sequencing involves using a mutant APOBEC3A enzyme as the conversion agent, which is described in more detail in the Reference Examples 1 to 4 below. This process is described in U.S. Provisional Application No. 63/328,444, which is incorporated herein by reference.

### TAPS

TET-assisted pyridine borane sequencing (TAPS) involves using a TET1 enzyme as the further agent and pyridine borane as the conversion agent. This process is described in Liu et al. (Nature Biotechnology, 2019, 37, pp. 424-429), which is incorporated herein by reference. The TET1 enzyme causes oxidation of 5-methylcytosine, 5-hydroxymethylcytosine and 5-formylcytosine in the target polynucleotide to 5-carboxylcytosine. Subsequent treatment with pyridine borane converts 5-carboxylcytosine (including residues that used to be 5-methylcytosine, 5-hydroxymethylcytosine and 5-formylcytosine) to dihydrouracil, but does not convert unmodified cytosine. Accordingly, TAPS allows identification of the modified cytosines 5-mC, 5-hmC, 5-fC and 5-caC by reading them as T/U; whereas unmodified cytosine is read as C.

### TAPSβ

TET-assisted pyridine borane sequencing with β-glucosyltransferase blocking (TAPSβ) involves using a T4 β-glucosyltransferase and a TET1 enzyme as the further agents, and pyridine borane as the conversion agent. This process is described in Liu et al. (Nature Communications, 2021, 12, 618), which is incorporated herein by reference. The T4 β-glucosyltransferase converts 5-hydroxymethylcytosine in the target polynucleotide to β-glucosyl-5-hydroxymethylcytosine, which prevents oxidation. The TET1 enzyme causes oxidation of 5-methylcytosine and 5-formylcytosine in the target polynucleotide to 5-carboxylcytosine. Subsequent treatment with pyridine borane converts 5-carboxylcytosine (including residues that used to be 5-methylcytosine and 5-formylcytosine) to dihydrouracil, but does not convert unmodified cytosine or β-glucosyl-5-hydroxymethylcytosine. Accordingly, TAPSβ allows identification of the modified cytosines 5-mC, 5-fC and 5-caC by reading them as T/U; whereas unmodified cytosine and 5-hmC are read as C.

### CAPS

Chemical-assisted pyridine borane sequencing (CAPS) involves using a potassium ruthenate (K₂RuO₄) as the further agent and 2-picoline borane as the conversion agent. This process is described in Liu et al. (Nature Communications, 2021, 12, 618), which is incorporated herein by reference. Potassium ruthenate causes oxidation of 5-hydroxymethylcytosine in the target polynucleotide to 5-formylcytosine. Subsequent treatment with 2-picoline borane converts 5-formylcytosine (including residues that used to be 5-hydroxymethylcytosine) and 5-carboxylcytosine to dihydrouracil, but does not convert unmodified cytosine or 5-methylcytosine. Accordingly, CAPS allows identification of the modified cytosines 5-hmC, 5-fC and 5-caC by reading them as T/U; whereas unmodified cytosine and 5-mC are read as C.

### PS

Pyridine borane sequencing (PS) involves using pyridine borane as the conversion agent. This process is described in Liu et al. (Nature Communications, 2021, 12, 618), which is incorporated herein by reference. Treatment with pyridine borane converts 5-formylcytosine and 5-carboxylcytosine to dihydrouracil, but does not convert unmodified cytosine, 5-methylcytosine or 5-hydroxymethylcytosine. Accordingly, PS allows identification of the modified cytosines 5-fC and 5-caC by reading them as T/U; whereas unmodified cytosine, 5-mC and 5-hmC are read as C.

### PS-c

Pyridine borane sequencing for 5-caC (PS-c) involves using O-ethylhydroxylamine as the further agent and pyridine borane as the conversion agent. This process is described in Liu et al. (Nature Communications, 2021, 12, 618), which is incorporated herein by reference. The O-ethylhydroxylamine converts 5-formylcytosine to an oxime derivative, which prevents 5-formylcytosine from converting to dihydrouracil. Subsequent treatment with pyridine borane converts 5-carboxylcytosine to dihydrouracil, but does not convert unmodified cytosine, 5-methylcytosine, 5-hydroxycytosine or the oxime derivative of 5-formylcytosine. Accordingly, PS-c allows identification of the modified cytosine 5-caC by reading it as T/U; whereas unmodified cytosine, 5-mC, 5-hmC and 5-fC are read as C.

### Methods of Sequencing

Also described herein is a method of sequencing polynucleotide sequences to detect modified cytosines, comprising:
preparing polynucleotide sequences for detection of modified cytosines using a method as described herein;
concurrently sequencing nucleobases in the first portion and the second portion; and
identifying modified cytosines by detecting differences when comparing a sequence output from the first portion with a sequence output from the second portion.

In one embodiment, sequencing is performed by sequencing-by-synthesis or sequencing-by-ligation.

In one embodiment, the step of preparing the polynucleotide sequences comprises using a selective processing method as described herein; and wherein the step of concurrent sequencing nucleobases in the first portion and the second portion is based on the intensity of the first signal and the intensity of the second signal.

In one embodiment, the method may further comprise a step of conducting paired-end reads.

In some embodiments, where the method comprises a step of selectively processing the at least one polynucleotide sequence comprising the first portion and the second portion, such that a proportion of first portions are capable of generating a first signal and a proportion of second portions are capable of generating a second signal, wherein the selective processing causes an intensity of the first signal to be greater than an intensity of the second signal, the data may be analysed using 16 QAM as mentioned herein. Accordingly, the step of concurrently sequencing nucleobases may comprise:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification represents a possible combination of respective first and second nucleobases; and
(d) based on the selected classification, base calling the respective first and second nucleobases.

In one embodiment, selecting the classification based on the first and second intensity data may comprise selecting the classification based on the combined intensity of the first and second signal components and the combined intensity of the third and fourth signal components.

In one embodiment, the plurality of classifications may comprise sixteen classifications, each classification representing one of sixteen unique combinations of first and second nucleobases.

In one embodiment, the first signal component, second signal component, third signal component and fourth signal component may be generated based on light emissions associated with the respective nucleobase.

In one embodiment, the light emissions may be detected by a sensor, wherein the sensor is configured to provide a single output based upon the first and second signals.

In one example, the sensor may comprise a single sensing element.

In one embodiment, the method may further comprise repeating steps (a) to (d) for each of a plurality of base calling cycles.

In some embodiments, where a proportion of first portions is capable of generating a first signal and a proportion of second portions is capable of generating a second signal, wherein an intensity of the first signal is substantially the same as an intensity of the second signal, the data may be analysed using 9 QAM as mentioned herein.

Accordingly, the step of concurrently sequencing nucleobases may comprise:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification of the plurality of classifications represents one or more possible combinations of respective first and second nucleobases, and wherein at least one classification of the plurality of classifications represents more than one possible combination of respective first and second nucleobases; and
(d) based on the selected classification, determining sequence information from the first portion and the second portion.

In one embodiment, selecting the classification based on the first and second intensity data may comprise selecting the classification based on the combined intensity of the first and second signal components and the combined intensity of the third and fourth signal components.

In one embodiment, when based on a nucleobase of the same identity, an intensity of the first signal component may be substantially the same as an intensity of the second signal component and an intensity of the third signal component is substantially the same as an intensity of the fourth signal component.

In one embodiment, the plurality of classifications may consist of a predetermined number of classifications.

In one embodiment, the plurality of classifications may comprise:
one or more classifications representing matching first and second nucleobases; and
one or more classifications representing mismatching first and second nucleobases, and
wherein determining sequence information of the first portion and second portion comprises:
   in response to selecting a classification representing matching first and second nucleobases, determining a match between the first and second nucleobases; or
   in response to selecting a classification representing mismatching first and second nucleobases, determining a mismatch between the first and second nucleobases.

In one embodiment, determining sequence information of the first portion and the second portion may comprise, in response to selecting a classification representing a match between the first and second nucleobases, base calling the first and second nucleobases.

In one embodiment, determining sequence information of the first portion and the second portion may comprise, based on the selected classification, determining that the second portion is modified relative to the first portion at a location associated with the first and second nucleobases.

In one embodiment, the first signal component, second signal component, third signal component and fourth signal component may be generated based on light emissions associated with the respective nucleobase.

In one embodiment, the light emissions may be detected by a sensor, wherein the sensor is configured to provide a single output based upon the first and second signals.

In one embodiment, the sensor may comprise a single sensing element.

In one embodiment, the method may further comprise repeating steps (a) to (d) for each of a plurality of base calling cycles.

### Kits

Methods as described herein may be performed by a user physically. In other words, a user may themselves conduct the methods of preparing polynucleotide sequences for detection of modified cytosines as described herein, and as such the methods as described herein may not need to be computer-implemented.

In another aspect of the invention, there is provided a kit comprising instructions for preparing polynucleotide sequences for detection of modified cytosines as described herein, and/or for sequencing polynucleotide sequences to detect modified cytosines as described herein.

### Computer Programs and Products

In other embodiments, methods as described herein may be performed by a computer. In other words, a computer may contain instructions to conduct the methods of preparing polynucleotide sequences for detection of modified cytosines as described herein, and as such the methods as described herein may be computer-implemented.

Accordingly, in another aspect of the invention, there is provided a data processing device comprising means for carrying out the methods as described herein.

The data processing device may be a polynucleotide sequencer.

The data processing device may comprise reagents used for synthesis methods as described herein.

The data processing device may comprise a solid support, such as a flow cell.

In another aspect of the invention, there is provided a computer program product comprising instructions which, when the program is executed by a processor, cause the processor to carry out the methods as described herein.

In another aspect of the invention, there is provided a computer-readable storage medium comprising instructions which, when executed by a processor, cause the processor to carry out the methods as described herein.

In another aspect of the invention, there is provided a computer-readable data carrier having stored thereon the computer program product as described herein.

In another aspect of the invention, there is provided a data carrier signal carrying the computer program product as described herein.

The various illustrative imaging or data processing techniques described in connection with the embodiments disclosed herein can be implemented as electronic hardware, computer software, or combinations of both. To illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative detection systems described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

Computer-executable instructions may be stored in a (transitory or non-transitory) computer readable storage medium (e.g., memory, storage system, etc.) storing code, or computer readable instructions.

### Additional Notes

The embodiments described herein are exemplary. Modifications, rearrangements, substitute processes, etc. may be made to these embodiments and still be encompassed within the teachings set forth herein. One or more of the steps, processes, or methods described herein may be carried out by one or more processing and/or digital devices, suitably programmed.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The term “comprising” may be considered to encompass “consisting”.

Disjunctive language such as the phrase “at least one of X, Y or Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value. The term “partially” is used to indicate that an effect is only in part or to a limited extent.

Unless otherwise explicitly stated, articles such as “a” or “an” should generally be interpreted to include one or more described items. Accordingly, phrases such as “a device configured to” or “a device to” are intended to include one or more recited devices. Such one or more recited devices can also be collectively configured to carry out the stated recitations. For example, “a processor to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

The present invention will now be described by way of the following non-limiting examples.

### EXAMPLES

### Reference Examples 1 to 4-Purification and Deaminase Activity of Mutant APOBEC3A Proteins

### Swal Assay Method

This assay was adapted and modified from Schutsky et al., Nucleic Acid Research, 45, 7655-7665, 2017. doi: 10.1093/nar/gkx345. Modifications to Schutsky et al. included the following. Instead of performing DNA precipitation and redissolving the DNA substrate into Swal compatible buffer, 1 μL of the altered cytidine deaminase APOBEC3A(Y130A) deamination reaction mixture was aliquoted into 9 μL Swal compatible buffer for restriction enzyme digestion for our gel assay. Appropriate controls were performed to determine the Swal restriction enzyme digestion efficiency was not compromised by the APOBEC reaction buffer. Instead of introducing 1.5-fold excess complementary strand prior to overnight Swal restriction enzyme digestion, 3-fold excess complementary strand was introduced. Instead of running the pre-heated 20% acrylamide/Tris-Borate-EDTA (TBE)/urea gel reported by Schutsky et al., the gel run was performed at room temperature with a 15% acrylamide/Tris-Borate-EDTA (TBE)/urea gel and observed good resolution between cut (deaminated) and uncut (unreacted) oligo substrates.

### Reference Example 1-Purification of APOBEC3A(Y130X) Mutant Proteins

The impact of all possible amino acid substitutions at position 130 of APOBEC3A on the deaminase activity of this enzyme was systematically assessed. To this end, 19 different His-tagged APOBEC3A constructs were cloned, each encoding a different amino acid at position 130 relative to the wild type tyrosine. The corresponding proteins were expressed in BL21(DE3) cells, purified using Ni-NTA agarose beads, and desalted/concentrated using spin columns to storage buffer (50 mM Tris pH 7.5, 200 mM NaCl, 5% (v/v) glycerol, 0.01% (v/v) Tween-20, 0.5 mM DTT). This yielded APOBEC3A(Y130X) mutant protein preparations with 80-85% purity, as judged by SDS-PAGE analysis.

### Reference Example 2-DNA Deaminase Activity of APOBEC3A(Y130X) Mutant Proteins

The deaminase activity of all purified APOBEC3A(Y130X) proteins was then analysed using the Swal assay, with a 37° C./2 hour reaction time and NEB APOBEC3A as positive control. 10-20 UM final concentration of Y130X recombinant enzymes were incubated with oLB1609 (C oligo, top panel) and oLB1612 (5mC oligo, bottom panel) at 37° C. for 2 hours. NEB APOBEC3A enzyme was purchased from NEBNext® Enzymatic Methyl-seq Kit (Catalog #E7120). Wild type APOBEC3A deaminated 5mC and C substrates to completion, consistent with previous literature. Different mutants exhibited a wide range of reactivities towards 5mC and C substrates, with some showing preference towards either substrate. Remarkably, APOBEC3A(Y130A) (first box) deaminated 5mC substrates almost completely (94.2%), while it deaminated the corresponding C substrate to a minor extent (29.4%). Other mutants, such as APOBEC3A(Y130P) and APOBEC3A(Y130T), also exhibited more complete deamination of the 5mC than C substrate, albeit to a lesser extent than APOBEC3A(Y130A). In contrast, APOBEC3A(Y130L) (second box) deaminated approximately half of the C substrate (56%), but almost none of the 5mC substrate (6.8%). The deaminase activity of all APOBEC3A(Y130X) mutants is quantified and summarised in the table below:

| | % C | % 5 mC | | % C | % 5 mC |
|---|---|---|---|---|---|
| Protein | deamination | deamination | Protein | deamination | deamination |
| NEB APOBEC | 92 | 94.9 | NEB APOBEC | 99.3 | 95.3 |
| Y130A | 29.4 | 94.2 | Y130V | 11.6 | 15 |
| Y130G | 3.7 | 19.1 | Y130D | 0.8 | 2.3 |
| Y130L | 58 | 6.8 | Y130E | 0 | 2.9 |
| Y130F | 84.6 | 94.3 | Y130S | 53.5 | 95.4 |
| Y130I | 8.1 | 8.1 | Y130C | 88.2 | 96.2 |
| Y130H | 83.4 | 95.8 | Y130W | 97.6 | 71.6 |
| Y130O | 1.6 | 14.6 | Y130P | 0.2 | 22.3 |
| Y130M | 37 | 55.4 | Y130R | 0.6 | 0.8 |
| Y130N | 3.6 | 9.1 | Y130T | 8 | 28.1 |
| Y130K | 0.3 | 0.9 | | | |

Because these Swal assays were performed as a single endpoint measurement (2 hour), it could be possible that the respective deamination reactions had already saturated. A time course analysis of APOBEC3A(Y130A) deaminase activity was therefore performed. The extent of C and 5mC deamination was monitored at 0, 5, 10, 30, 60 and 120 minutes by incubation of ˜10-20 UM of APOBEC(Y130A) with 500 nM C and 5mC oligonucleotide substrate. A greater difference in the extent of 5mC versus C deamination was observed at t≤30 min.

The kinetics of deamination by wild type APOBEC3A and mutant APOBEC3A(Y130A) were quantitatively compared. The initial deamination reaction velocity was measured at a range of DNA substrate concentrations and used to construct Michaelis-Menten curves for 5mC and C substrates, respectively. The resulting Km and Kcat values were then derived from these data. The catalytic efficiency of APOBEC3A(Y130A) was ˜100-fold higher on 5mC than C substrates corroborating the endpoint Swal assays shown above.

### Reference Example 3-Purification of APOBEC3A(Y130A-Y132H) Double Mutant Protein

APOBEC3A(Y130A-Y132H) protein was expressed in BL21(DE3) cells, purified using Ni-NTA agarose beads, and desalted/concentrated using spin columns to storage buffer (50 mM Tris pH 7.5, 200 mM NaCl, 5% (v/v) glycerol, 0.01% (v/v) Tween-20, 0.5 mM DTT). This yielded APOBEC3A(Y130A-Y132H) mutant protein preparations with 90-95% purity, as judged by SDS-PAGE analysis.

### Reference Example 4-DNA Deaminase Activity of APOBEC3A(Y130A-Y132H) Double Mutant Protein

The deaminase activity of purified APOBEC3A(Y130A-Y132H) double mutant protein was then analyzed using the Swal assay, with a 37° C./2 hour reaction time and NEB APOBEC3A as positive control. The conditions used were the same as described in Reference Example 2 with the exception that the Swal assay used reaction conditions of 40 mM sodium acetate pH 5.2, 37° C. for 1 hour to 16 hours. The DNA substrates are shown below:

5′ GAGGTGTATGGTTGTACTAAT/5mC/ACT/5mC/CTGGA/5mC/GAATCTTAA/5mC/ACAA/5mC/
GTGCAG/5mC/CAAA/5mC/GCTT/5mC/GC/5mC/ACGG/5mC/AACGTG/5mC/GGACT/5mC/GTCG/
5mC/CTTA/5mC/AATCG/5mC/GCAGGT/5mC/ACGTTGAAGATGAGGATG-3′
GAGGTGTATGGTTGTAG/5mC/GCAAATCGTAAAA/5mC/GCAAAGCGAAAAC/5mC/GCAAACCGTAAA
C/5mC/GAAAAGCGCTTGAAGATGAGGATG
GAGGTGTATGGTTGTAG/5mC/GGAAAACGGAAAT/5mC/GGAAAACGTAAAG/5mC/GTAAATCGGAAA
G/5mC/GAAAAGCGGTTGAAGATGAGGATG
GAGGTGTATGGTTGTAA/5mC/GTAAACCGCAAAC/5mC/GGAAAACGAAAAT/5mC/GCAAACCGAAAA
C/5mC/GTAAAACGCTTGAAGATGAGGATG
GAGGTGTATGGTTGTAA/5mC/GAAAACCGGAAAT/5mC/GAAAAGCGTAAAT/5mC/GTAAATCGCAAA
A/5mC/GGAAATCGATTGAAGATGAGGATG

After the deaminase reaction the deaminated oligo substrates were PCR-amplified, sequenced, and the number of C and 5mC deamination events per read were counted. APOBEC3A(Y130A-Y132H) exhibited higher levels of deamination at all methylated sites compared to unmethylated sites. This was consistent across both CpG and non-CpG contexts, and was robust to variation in reaction time. The difference in deamination level between methylated and unmethylated sites was markedly higher for APOBEC3A(Y130A-Y132H) than APOBEC3A(Y130A), indicating that APOBEC3A(Y130A-Y132H) achieves better discrimination of methylated sites than APOBEC3A(Y130A). In addition, APOBEC3A(Y130A-Y132H) deaminated methylated sites more efficiently than unmethylated sites across all xCpGx motifs.

### Example 1-Methylation Analysis on Methylated pUC19 Sample Using 9 QaM

### Oligo Sequences:

Asterisk (*) indicates a phosphorothioate linkage.

Underline indicates 5-methylcytosine instead of cytosine (in “P5_BbvCI_P7-methylated” and “BspQI_iSce_Loop-methylated”, all cytosines are replaced with 5-methylcytosines to prevent unwanted conversion of cytosine to uracil in the adaptor sequence during bisulfite conversion).

Bold indicates nicking restriction site (or its complement) of Nt.BspQI, which recognises the following sequence (nicking site is indicated by arrow):

5′...GCTCTTCN^{▾}...3′
3′...CGAGAAGN...5′

### [Biotin-T] Indicates the Following Structure:

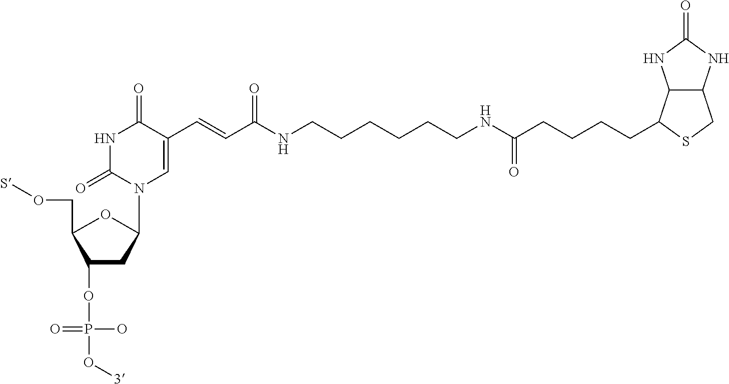

P5_BbvCI_P7 (SEQ ID NO. 67):
GCTGAGGATCTCGTATGCCGTCTTCTGCTTGUAATGATACGGCGACCACCGAGATCTACACTCC
TCAGC*T
BspQI_iSce_Loop (SEQ ID NO. 68):
GAAGAGCACACGTCTGAACTCCAGTCACTAGGGA[Biotin-T]AACAGGGTAATCTTTCCCTA
CACGACGCTCTTC*T
P5_BbvCI_P7-methylated (SEQ ID NO. 69):
GCTGAGGATCTCGTATGCCGTCTTCTGCTTGUAATGATACGGCGACCACCGAGATCTACACTCC
TCAGC*T
BspQI_iSce_Loop-methylated (SEQ ID NO. 70):
GAAGAGCACACGTCTGAACTCCAGTCACTAGGGA[Biotin-T]AACAGGGTAATCTTTCCCTA
CACGACGCTCTTC*T

### Adaptor Annealing:

1. A mixture of 4 μl of 100 μM P5_BbvCI_P7-methylated oligo, 11 μl water, 2 μl 10× TEN buffer (Illumina) and 3 μl IDTE buffer was heated to 98C for 30s, then a slow cool to room temperature (eg. 0.1C/s ramp down to RT). This gives a 20 μM stock of annealed P5_BbvCI_P7-methylated adaptor.
2. Separately, a mixture of 4 μl of 100 μM BspQI_iSce_Loop-methylated oligo, 11 μl water, 2 μl 10× TEN buffer (Illumina) and 3 μl IDTE buffer was heated to 98C for 30s, then a slow cool to room temperature (eg. 0.1C/s ramp down to RT). This gives a 20 μM stock of annealed BspQI_iSce_Loop-methylated adaptor.
3. Equal volumes of the 20 μM stock of annealed P5_BbvCI_P7-methylated adaptor from step 1 and 20 μM stock of annealed BspQI_iSce_Loop-methylated adaptor from step 2 are mixed together, giving a stock solution with 10 μM each of annealed P5_BbvCI_P7-methylated adaptor and annealed BspQI_iSce_Loop-methylated adaptor.

### Preparation of Library:

1. NEB Ultra II FS reagents were thawed at room temperature and kept on ice until use.
2. The Ultra II FS Enzyme mix was vortexed for 5-8 seconds prior to use and placed on ice.
3. In a 0.2 ml PCR tube on ice, 26 μl DNA (100 ng of input DNA (methylated pUC19 sample) diluted to 26 μl with Milli-Q grade water), 7 μl of NEBNext Ultra II FS Reaction Buffer and 2 μl of NEBNext Ultra II FS Enzyme Mix were added, briefly vortexed and spun in a microcentrifuge to mix.
4. In a Thermocycler with the heated lid set to 75C, the tubes were incubated for 5 mins at 37C, then 30 mins at 65C then held at 4C.
5. The following were added to the FS reaction mixture from step 4: 30 μl of NEBNext Ultra II Ligation Master Mix, 1 μl of NEBNext Ligation Enhancer and 2.5 μl of the loop adaptors P5_BbvCI_P7-methylated and BspQI_iSce_Loop-methylated (10 μM each) prepared from step 3 of “Adaptor annealing”.
6. The entire volume was pipetted up and down 10× to mix, followed by a brief spin in a microcentrifuge.
7. The mixture was incubated at 20C for 15 mins in a thermocycler with the heated lid off.
8. 3 μl of USER Enzyme (NEB) was added to the ligation mix.
9. The mixture was mixed well and incubated at 37 C for 15 mins with the heated lid set to >47C.
10. Adaptor ligated DNA was then size selected via a 0.8× SPRI (iTune beads) selection: 57 μl iTune beads (ILMN) were added to 68.5 μl of ligation reaction, mixed and incubated at RT for 5 mins.
11. The mixture was placed on a magnet for 5 mins, and the supernatant was discarded.
12. The beads were washed twice with 200 μl of 80% ethanol-200 μl 80% ethanol was added with beads on the magnet, followed by a 30s wait, and ethanol was removed, then the wash was repeated once more.
13. The last remnants of ethanol were removed with a P10 pipette and tip.
14. Beads were then air dried for 5 mins.
15. DNA was eluted from beads with 40 μl of 0.1× TE buffer. At this stage, 20 μl was saved as a “non-converted” control, the remaining 20 μl was treated to bisulfite conversion, following the Zymo Research EZ-96 DNA Methylation Gold MagPrep kit (steps 16-25 are taken from the instructions for this kit).
16 In a 0.2 ml PCR tube, 20 μl of 0.8× SPRI selected ligation and 130 μl of CT Conversion Reagent (comprises sodium metabisulfite) were added.
17. The mixture was incubated on a thermocycler at 98C for 10 mins, then 64C for 2.5 hours, followed by holding at 4C for up to 20 hours.
18 The sample was transferred to 1.7 ml tubes for subsequent steps. 600 μl of M-Binding Buffer and 10 μl of MagBinding Beads were added. The mixture was vortexed for 30s.
19 Incubate at RT for 5 mins, then place on a magnet for 5 mins.
20 The supernatant was removed and discarded. 400 μl of M-Wash buffer was added to the beads, and then vortexed for 30s. The mixture was placed back on magnet until the beads pelleted.
21. The supernatant was removed and discarded.
22. 200 μl of M-Desulphonation Buffer was added to the beads, and then vortexed for 30s. The mixture was incubated at RT for 15-20 mins. The mixture was then placed back on magnet until beads pelleted.
23. The supernatant was removed and discarded. 400 μl of M-Wash buffer was added to the beads, then vortexed for 30s. The mixture was placed back on magnet until beads pelleted. This wash step was repeated once.
24. The supernatant after 2nd wash was removed, and the tubes were transferred to a hot block at 55C to air dry the beads for 20-30 mins and remove residual M-Wash buffer.
25. 25 μl of M-Elution Buffer was added to the dried beads and vortexed for 30s. The elution mixture was heated at 55C for 4 mins then the tubes were placed back on the magnet for 1 min (or until the beads pelleted). The eluate was removed and transferred to a new 1.7 ml tube.
26. 175 μl of HT1 buffer (ILMN Hybridisation buffer) and 10 μl of HT1 washed MyOne Streptavidin T1 beads (Thermofisher) were added. The tubes were incubated on a rocker at RT for 30 mins. (This step selects for material which has the biotinylated loop adaptor, and removes the material which has the P5/P7 adaptors on both ends).
27. The tubes were placed on a magnet until the beads pelleted.
28 The beads were washed twice with 200 μl of Tagmentation Wash Buffer (TWB, Illumina).
29 The beads were then washed once with 200 μl of Resuspension Buffer (RSB, Illumina).
30 The beads were resuspended in 20 μl of Milli-Q grade water and transferred to 0.2 ml tubes for the final PCR.
31. 20 μl of beads+DNA were combined with 25 μl of Q5U Mastermix (NEB) and 5 μl of PPC (PCR Primer Cocktail, Illumina).
32. The mixture was amplified by PCR: cycling procedure—98C for 3 min followed by 12 cycles of (98C 45 s, 60C 2 min, 68C 2 min), then 68C for 5 mins and then hold at 4C.
33 PCR products were analysed by TapeStation D1000 (Agilent), and then subjected to a further SPRI clean-up before quantification using a Qubit Broad Range dsDNA assay kit (Thermofisher).

### Sequencing:

### Sequencing was Conducted on the MiniSeq.

1. 400 μl BspQI mix was made up-360 μl Milli-Q grade water, 40 μl of rNEB3.1 buffer (NEB) and 8 μl of Nt.BspQI (NEB were combined). The mixture was vortexed to mix and briefly spun down. The mixture was pipetted into the “EXT” position of the MiniSeq cartridge (position to the left of the Custom Primer positions).
2. The library was denatured (0.1N NaOH) and diluted to 0.5 pM final concentration in HT1 buffer according to Illumina protocol. 500 μl was loaded into the “Library” position of the MiniSeq cartridge.
3. Setup was run using MiniSeq Control Software, using a standard MiniSeq run.
4. For a CA dye swap, standard IMX was removed from the IMX position of the MiniSeq cartridge, then the position was washed 5 times with Milli-Q grade water, and replaced with 20 mls of custom IMX, where the standard two-dye system for A (A represented by red and green) and one-dye system for C (C represented by red) is replaced with a two-dye system for C (C represented by red and green) and one-dye system for A (A represented by red).

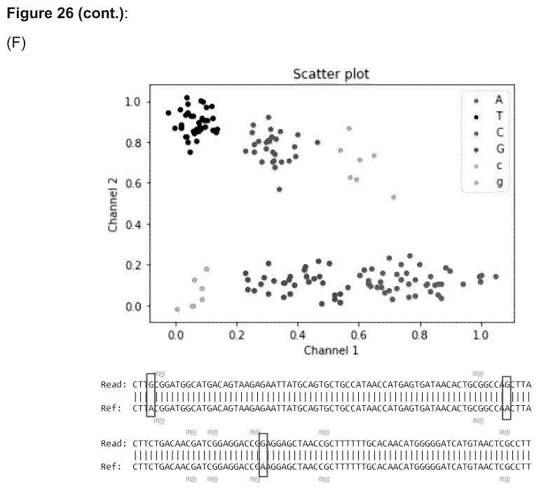

The 9 QaM results are shown in FIGS. 26A to 26F for six different library fragments, where modified cytosines can be identified by characteristic clouds in the top right corner and the bottom left corner in the plot. If the original strands in the library contained a (5mC)-G base pair (the first base corresponding to the forward strand of the library polynucleotide, and the second base corresponding to the reverse strand of the library polynucleotide), this corresponds to a C-G base pair after bisulfite conversion. As such, the forward strand of the template provides a C read (as the forward strand of the template has a G at the corresponding position), and the reverse complement strand of the template provides a C read too (as the reverse complement strand of the template has a G at the corresponding position too), which therefore appears in the top right corner of the plots in FIGS. 26A to 26F (a (C,C) read).

In addition, if the original strands in the library contained a G-(5mC) base pair (the first base corresponding to the forward strand of the library polynucleotide, and the second base corresponding to the reverse strand of the library polynucleotide), this corresponds to a G-C base pair after bisulfite conversion. As such, the forward strand of the template provides a G read (as the forward strand of the template has a C at the corresponding position), and the reverse complement strand of the template provides a G read too (as the reverse complement strand of the template has a C at the corresponding position too), which therefore appears in the bottom left corner of the plots in FIGS. 26A to 26F (a (G,G) read).

By contrast, if the original strands in the library contained a C-G base pair (the first base corresponding to the forward strand of the library polynucleotide, and the second base corresponding to the reverse strand of the library polynucleotide), this corresponds to a T-G mismatched base pair after bisulfite conversion (where C is converted to U, and U is read as T). As such, the forward strand of the template provides a T read (as the forward strand of the template has an A at the corresponding position), and the reverse complement strand of the template provides a C read (as the reverse complement strand of the template has a G at the corresponding position), which therefore appears in the top middle portion of the plots in FIGS. 26A to 26F (a (T,C) read).

If the original strands in the library contained a G-C base pair (the first base corresponding to the forward strand of the library polynucleotide, and the second base corresponding to the reverse strand of the library polynucleotide), this corresponds to a G-T mismatched base pair after bisulfite conversion (where C is converted to U, and U is read as T). As such, the forward strand of the template provides a G read (as the forward strand of the template has a C at the corresponding position), and the reverse complement strand of the template provides an A read (as the reverse complement strand of the template has a T at the corresponding position), which therefore appears in the bottom middle portion of the plots in FIGS. 26A to 26F (a (G,A) read).

If the original strands in the library contained a T-A base pair (the first base corresponding to the forward strand of the library polynucleotide, and the second base corresponding to the reverse strand of the library polynucleotide), this remains as a T-A base pair after bisulfite conversion. As such, the forward strand of the template provides a T read (as the forward strand of the template has an A at the corresponding position), and the reverse complement strand of the template provides a T read too (as the reverse complement strand of the template has an A at the corresponding position too), which therefore appears in the top left corner of the plots in FIGS. 26A to 26F (a (T,T) read).

Finally, if the original strands in the library contained an A-T base pair (the first base corresponding to the forward strand of the library polynucleotide, and the second base corresponding to the reverse strand of the library polynucleotide), this remains as an A-T base pair after bisulfite conversion. As such, the forward strand of the template provides an A read (as the forward strand of the template has a T at the corresponding position), and the reverse complement strand of the template provides an A read too (as the reverse complement strand of the template has a T at the corresponding position too), which therefore appears in the bottom right corner of the plots in FIGS. 26A to 26F (an (A,A) read).

| Library | Accuracy | Sensitivity | Specificity |
|---|---|---|---|
| Library fragment 1 | 85/85 | 10/10 | 75/75 |
| (FIG. 26A) | (100%) | (100%) | (100%) |
| Library fragment 2 | 72/72 | 10/10 | 62/62 |
| (FIG. 26B) | (100%) | (100%) | (100%) |
| Library fragment 3 | 73/73 | 10/10 | 63/63 |
| (FIG. 26C) | (100%) | (100%) | (100%) |
| Library fragment 4 | 148/150 | 17/18 | 133/133 |
| (FIG. 26D) | (98.67%) | (94.44%) | (100%) |
| Library fragment 5 | 148/150 | 14/14 | 136/136 |
| (FIG. 26E) | (98.67%) | (100%) | (100%) |
| Library fragment 6 | 147/150 | 14/14 | 136/136 |
| (FIG. 26F) | (98%) | (100%) | (100%) |

(Accuracy = number of correct base calls (GCAT, irrespective of methylation status)/total number of bases; Sensitivity = number of true positive methylated base calls/total number of methylated bases; Specificity = number of true negative methylated base calls/(number of true negative methylated base calls + number of false positive methylated base calls))

Overall, these results show that methylation analysis can be conducted on polynucleotide sequences to identify modified cytosines. In particular, by enabling concurrent sequencing of the forward and reverse complement strands of the template (or reverse and forward complement strands of the template), modified cytosines can be identified quickly and accurately.

SEQUENCE LISTING

SEQ ID NO. 1: P5 sequence
AATGATACGGCGACCACCGAGATCTACAC
SEQ ID NO. 2: P7 sequence
CAAGCAGAAGACGGCATACGAGAT
SEQ ID NO. 3: P5′ sequence (complementary to P5)
GTGTAGATCTCGGTGGTCGCCGTATCATT
SEQ ID NO. 4: P7′ sequence (complementary to P7)
ATCTCGTATGCCGTCTTCTGCTTG
SEQ ID NO. 5: Alternative P5 sequence
AATGATACGGCGACCGA
SEQ ID NO. 6: Alternative P5′ sequence (complementary to alternative P5 sequence)
TCGGTCGCCGTATCATT
SEQ ID NO: 7: UniProt Q9GZX7
MDSLLMNRRKFLYQFKNVRWAKGRRETYLCYVVKRRDSATSFSLDFGYLRNKNGCHVELLFLRY
ISDWDLDPGRCYRVTWFTSWSPCYDCARHVADFLRGNPNLSLRIFTARLYFCEDRKAEPEGLRR
LHRAGVQIAIMTFKDYFYCWNTEVENHERTFKAWEGLHENSVRLSRQLRRILLPLYEVDDLRDA
FRTLGL
SEQ ID NO: 8: UniProt G3QLD2
MDSLLMNRRKFLYQFKNVRWAKGRRETYLCYVVKRRDSATSFSLDFGYLRNKNGCHVELLFLRY
ISDWDLDPGRCYRVTWFTSWSPCYDCARHVADFLRGNPNLSLRIFTARLYFCEDRKAEPEGLRR
LHRAGVQIAIMTEKENHERTFKAWEGLHENSVRLSRQLRRILLPLYEVDDLRDAFRTLGL
SEQ ID NO: 9: Uniprot Q9WVE0
MDSLLMKQKKFLYHFKNVRWAKGRHETYLCYVVKRRDSATSCSLDFGHLRNKSGCHVELLFLRY
ISDWDLDPGRCYRVTWFTSWSPCYDCARHVAEFLRWNPNLSLRIFTARLYFCEDRKAEPEGLRR
LHRAGVQIGIMTFKDYFYCWNTEVENRERTFKAWEGLHENSVRLTRQLRRILLPLYEVDDLRDA
FRMLGF
SEQ ID NO: 10: UniProt P41238
MTSEKGPSTGDPTLRRRIEPWEFDVFYDPRELRKEACLLYEIKWGMSRKIWRSSGKNTTNHVEV
NFIKKFTSERDFHPSMSCSITWFLSWSPCWECSQAIREFLSRHPGVTLVIYVARLFWHMDQQNR
QGLRDLVNSGVTIQIMRASEYYHCWRNFVNYPPGDEAHWPQYPPLWMMLYALELHCIILSLPPC
LKISRRWQNHLTFFRLHLQNCHYQTIPPHILLATGLIHPSVAWR
SEQ ID NO: 11: NCBI XP_030856728.1
MSRKIWRSSGKNTTNHVEVNFIKKFTSERHFHPSISCSITWFLSWSPCWECSQAIREFLSQHPG
VTLVIYVARLFWHMDQQNRQGLRDLVNSGVTIQIMRASEYYHCWRNFVNYPPGDEAHWPQYPPL
WMMLYALELHCIILSLPPCLKISRRWQNHLTFFRLHLQNCHYQTIPPHILLATGLIHPSVAWR
SEQ ID NO: 12: Uniprot P51908
MSSETGPVAVDPTLRRRIEPHEFEVFFDPRELRKETCLLYEINWGGRHSVWRHTSQNTSNHVEV
NFLEKFTTERYFRPNTRCSITWFLSWSPCGECSRAITEFLSRHPYVTLFIYIARLYHHTDQRNR
QGLRDLISSGVTIQIMTEQEYCYCWRNFVNYPPSNEAYWPRYPHLWVKLYVLELYCIILGLPPC
LKILRRKQPQLTFFTITLQTCHYQRIPPHLLWATGLK
SEQ ID NO: 13: UniProt Q9Y235
MAQKEEAAVATEAASQNGEDLENLDDPEKLKELIELPPFEIVTGERLPANFFKFQFRNVEYSSG
RNKTFLCYVVEAQGKGGQVQASRGYLEDEHAAAHAEEAFENTILPAFDPALRYNVTWYVSSSPC
AACADRIIKTLSKTKNLRLLILVGRLFMWEEPEIQAALKKLKEAGCKLRIMKPQDFEYVWQNFV
EQEEGESKAFQPWEDIQENFLYYEEKLADILK
SEQ ID NO: 14: Uniprot G3SGN8
MAQKEEAAAATEAAAATEAASQNGEDLENLDDPEKLKELIELPPFEIVTGERLPANFFKFQFRN
VEYSSGRNKTFLCYVVEAQGKGGQVQASRGYLEDEHAAAHAEEAFFNTILPAFDPALRYNVTWY
VSSSPCAACADRIIKTLSKTKNLRLLILVGRLFMWEEPEIQAALKKLKEAGCKLRIMKPQDFEY
VWQNFVEQEEGESKAFQPWEDIQENFLYYEEKLADILK
SEQ ID NO: 15: Uniprot Q9WV35
MAQKEEAAEAAAPASQNGDDLENLEDPEKLKELIDLPPFEIVTGVRLPVNFFKFQFRNVEYSSG
RNKTFLCYVVEVQSKGGQAQATQGYLEDEHAGAHAEEAFFNTILPAFDPALKYNVTWYVSSSPC
AACADRILKTLSKTKNLRLLILVSRLFMWEEPEVQAALKKLKEAGCKLRIMKPQDFEYIWQNFV
EQEEGESKAFEPWEDIQENFLYYEEKLADILK
SEQ ID NO: 16: UniProt P31941
MEASPASGPRHLMDPHIFTSNFNNGIGRHKTYLCYEVERLDNGTSVKMDQHRGFLHNQAKNLLC
GFYGRHAELRFLDLVPSLQLDPAQIYRVTWFISWSPCFSWGCAGEVRAFLQENTHVRLRIFAAR
IYDYDPLYKEALQMLRDAGAQVSIMTYDEFKHCWDTFVDHQGCPFQPWDGLDEHSQALSGRLRA
ILQNQGN
SEQ ID NO: 17: GenBank XP_045219544.1
MDGSPASRPRHLMDPNTFTFNFNNDLSVRGRHQTYLCYEVERLDNGTWVPMDERRGFLHNKAKN
VPCGDYGCHVELRFLCEVPSWQLDPAQTYRVTWFISWSPCFRKGCAGQVRAFLQENKHVRLRIF
AARIYDYDPRYQEALRTLRDAGAQVSIMTYEEFKHCWDTFVDRQGRPFQPWDGLDEHSQALSGR
LRAILQNQGN
SEQ ID NO: 18: GenBank AER45717.1
MEASPASGPRHLMDPCVFTSNFNNGIRWHKTYLCYEVERLDNGTWVKMDQHRGFLHNQARNPLY
GLDGRHAELRFLGLLPYWQLDPAQIYRVTWFISWSPCFSWGCARQVRAFLQENTHVRLRIFAAR
IYDYDPLYKEALQMLRDAGAQVSIMTYDEFEYCWNTFVDHQGCPFQPWDGLEEHSQALSGKLQA
ILLNQGN
SEQ ID NO: 19: GenBank XP_003264816.1
MEASPASRPGHLMDPQVFTSNENNGIRWHKTYLCYEVERLDNGTWVKMDQHRGFLHNQAKNLFC
GFYGRHAELCFLDLVPSLQLDPAQTYRVTWFISWSPCFSWGCAEQVRAFLQENTHVRLRLFAAR
IYDYDPLYKEALQMLRGAGAQVSIMTYHEFKHCWDTFVDHQGRPFQPWDGLEEHSQALSGRLQA
ILQNQGN
SEQ ID NO: 20: GenBank PNI48846.1
TEASPASGPRHLMDPHIFTSNENNGIGRRKTYLCYEVERLDNGTSVKMDQHRGFLHNQAKNLLC
GFYGRHAELCFLDLVPSLQLDPAQIYRVTWFISWSPCFSWGCAGQVRAFLQENTHVRLRIFAAR
IYDYDPLYKEALQMLRDAGAQVSIMTYDEFKHCWDTFVDHQGCPFQPWDGLEEHSQALSGRLRA
ILQNQGN
SEQ ID NO: 21: GenBank ADO85886.1
VEASPASGPRHLMDPHIFTSNFNNVIGRHKTYLCYEVERLDNGTWVKMDQHRGFLHNQAKNLLC
GFYGRHAELRFLDLVPSLQLDPAQIYRVTWFISWSPCFSWGCAGQVRAFLQENTHVRLHIFAAR
IYDYDPLYKEALQMLRDAGAQVSIMTYDEFKHCWDTFVDHQGCPFQPWDGLEEHSQALSGRLRA
ILQNQGN
SEQ ID NO: 22: UniProt Q9UH17
MNPQIRNPMERMYRDTFYDNFENEPILYGRSYTWLCYEVKIKRGRSNLLWDTGVFRGQVYFKPQ
YHAEMCFLSWFCGNQLPAYKCFQITWFVSWTPCPDCVAKLAEFLSEHPNVTLTISAARLYYYWE
RDYRRALCRLSQAGARVTIMDYEEFAYCWENFVYNEGQQFMPWYKFDENYAFLHRTLKEILRYL
MDPDTFTFNFNNDPLVLRRRQTYLCYEVERLDNGTWVLMDQHMGFLCNEAKNLLCGFYGRHAEL
RFLDLVPSLQLDPAQIYRVTWFISWSPCFSWGCAGEVRAFLQENTHVRLRIFAARIYDYDPLYK
EALQMLRDAGAQVSIMTYDEFEYCWDTFVYRQGCPFQPWDGLEEHSQALSGRLRAILQNQGN
SEQ ID NO: 23: Uniprot G3QV16
MNPQIRNPMERMYRGTFYNNFENEPILYGRSYNWLCYEVKIKRGRSNLLWNTGVFRGQMYSQPE
HHAEMCFLSWFCGNQLPAYKCFQITWFVSWTPCPDCVAKLAEFLAEYPNVTLTISTARLYYYWE
RDYRRALCRLSQAGARMKIMDYEECAYCWENFVYKEGQQFMPWYKFDENYAFLHHTLKEILRHL
MDPDTFTFNFNNDPLVLRRHQTYLCYEVERLDNGTWVLMDRHMGFLCNEAKNLLCGFYGRHAEL
RFLDLVPSLQLDPAQIYRVTWFISWSPCFSWGCAGQVCEFLQENTHMRLRIFAARIYDYDPLYK
KALQMLRDAGAQVSIMTYDEFKHCWDTFVYRQGCPFQPWDGLEEHSQALSGRLQAILQNQGN
SEQ ID NO: 24: Uniprot F6M3K5
MNPQIRNPMERMYRRTFNYNFENEPILYGRSYTWLCYEVKIRKDPSKLPWDTGVFRGQMYSKPE
HHAEMCFLSWFCGNQLPAHKRFQITWFVSWTPCPDCVAKVAEFLAEYPNVTLTISAARLYYYWE
TDYRRALCRLRQAGARVKIMDYEEFAYCWENFVYNEDQSFMPWYKFDDNYAFLHHKLKEILRHL
MDPDTFTSNFNNDLSVLGRHQTYLCYEVERLDNGTWVPMDQHWGFLCNQAKNVPRGDYGCHAEL
CFLDQVSSWQLDPAQTYRVTWFISWSPCFSWGCADQVYAFLQENTHVRLRIFAARIYDYNPLYQ
EALRTLRDAGAQVSIMTYDEFEYCWDTFVDRQGRPFQPWDGLDEHSQALSGRLRAILQNQGN
SEQ ID NO: 25: UniProt Q9NRW3
MNPQIRNPMKAMYPGTFYFQFKNLWEANDRNETWLCFTVEGIKRRSVVSWKTGVFRNQVDSETH
CHAERCFLSWFCDDILSPNTKYQVTWYTSWSPCPDCAGEVAEFLARHSNVNLTIFTARLYYFQY
PCYQEGLRSLSQEGVAVEIMDYEDFKYCWENFVYNDNEPFKPWKGLKTNFRLLKRRLRESLQ
SEQ ID NO: 26: Uniprot Q694B5
MNPQIRNPMKAMYPGTFYFQFKNLWEANDRNETWLCFTVEGIKRRSVVSWKTGVFRNQVDSETH
CHAERCFLSWFCDDILSPNTNYQVTWYTSWSPCPECAGEVAEFLARHSNVNLTIFTARLYYFQD
TDYQEGLRSLSQEGVAVKIMDYKDFKYCWENFVYNDDEPFKPWKGLKYNFRFLKRRLQEILE
SEQ ID NO: 27: Uniprot B0LW74
MNPQIRNPMKAMDPGTFYFQFKNLWEANNRNETWLCFTVEVIKQHSTVSWETGVFRNQVDLETH
CHAERCFLSWFCEDILSPNTDYQVTWYTSWSPCLDCAGEVAKFLARHNNVMLTIYTARLYYSQY
PNYQQGLRSLSEKGVSVKIMDYEDFKYCWEKFVYDDGEPFKPWKGLKTSFRFLKRRLREILQ
SEQ ID NO: 28: UniProt Q96AK3
MNPQIRNPMERMYRDTFYDNFENEPILYGRSYTWLCYEVKIKRGRSNLLWDTGVFRGPVLPKRQ
SNHRQEVYFRFENHAEMCFLSWFCGNRLPANRRFQITWFVSWNPCLPCVVKVTKFLAEHPNVTL
TISAARLYYYRDRDWRWVLLRLHKAGARVKIMDYEDFAYCWENFVCNEGQPFMPWYKEDDNYAS
LHRTLKEILRNPMEAMYPHIFYFHFKNLLKACGRNESWLCFTMEVTKHHSAVERKRGVERNQVD
PETHCHAERCFLSWFCDDILSPNTNYEVTWYTSWSPCPECAGEVAEFLARHSNVNLTIFTARLC
YFWDTDYQEGLCSLSQEGASVKIMGYKDFVSCWKNFVYSDDEPFKPWKGLQTNFRLLKRRLREI
LQ
SEQ ID NO: 29: NCBI NP_001332895.1
MNPQIRNPMERMYRRTFYNHFENEPILYGRSYTWLCYEVKIKRGCSNLIWDTGVFRGPVLPKLQ
SNHRQEVYFQFENHAEMCFFSWFCGNRLPANRRFQITWFVSWNPCLPCVVKVTKFLAEHPNVTL
TISAARLYYYQDREWRRVLRRLHKAGARVKIMDYKDFAHCWENFVYNEGQQFMPWYKEDDNYAS
LHRTLKEILRNPMEAMYPHVFYFHFKNLLKACGRNESWLCFTVDVTEHHPPVSWKRGVFRNPVD
PETHCHAERCFLSWFCDDILSPNTNYQVTWYTSWSPCPECAREVAEFLARHSNVKLTIFTARLY
HFWNTDYQEGLCSLSQEGASVKIMSYKDFVSCWKNFVYSDDEPFKPWKGLKTNFRLLKTMLREI
LQ
SEQ ID NO: 30: NCBI NP_001332931.1
MNPQIRNPMERMYRRTFNYNFENEPILYGRSYTWLCYEVKIRKDPSKLPWDTGVFRGQVYFQPQ
YHAEMCFLSWFCGNQLPAYKRFQITWFVSWNPCPDCVAKVTEFLAEHPNVTLTISVARLYYYRG
KDWRRALCRLHQAGARVKIMDYEEFAYCWENFVYNEGQSFMPWDKFDDNYAFLHHKLKEILRNP
MKAMYPHTFYFHFENLQKAYGRNETWLCFAVEIIKQHSTVPWKTGVFRNQVDPETHCHAERCEL
SWFCDNTLSPKKNYQVTWYISWSPCPECAGEVAEFLATHSNVKLTIYTARLYYFWDTDYQEGLR
SLSEEGASMEIMGYEDFKYCWENFVYNDGEPFKPWKGINTNFRFLERRLWKILQ
SEQ ID NO: 31: UniProt Q8IUX4
MKPHFRNTVERMYRDTFSYNFYNRPILSRRNTVWLCYEVKTKGPSRPRLDAKIFRGQVYSQPEH
HAEMCFLSWFCGNQLPAYKCFQITWFVSWTPCPDCVAKLAEFLAEHPNVTLTISAARLYYYWER
DYRRALCRLSQAGARVKIMDDEEFAYCWENFVYSEGQPFMPWYKFDDNYAFLHRTLKEILRNPM
EAMYPHIFYFHFKNLRKAYGRNESWLCFTMEVVKHHSPVSWKRGVFRNQVDPETHCHAERCELS
WFCDDILSPNTNYEVTWYTSWSPCPECAGEVAEFLARHSNVNLTIFTARLYYFWDTDYQEGLRS
LSQEGASVEIMGYKDFKYCWENFVYNDDEPFKPWKGLKYNFLFLDSKLQEILE
SEQ ID NO: 32: Uniprot G3RD21
MKPQFRNTVERMYRGTFSYNENNRPILSRRNTVWLCYEVKTKGPSRPPLDAKIFRGQVYFQPQY
HAEMCFLSWFCGNQLPAYKCFQITCFVSWTPCPDCVAKLAEFLAEHPNVTLTISAARLYYYWER
DYRRALRRLRQAGARVKIMDDEEFAYCWENFVYSEGQPFMPWHKFDDNYAFLHRTLKEILRNPM
EAMYPHIFYFHFKNLLKAYGRNESWLCFTMEVIKHHSPVSWKRGVFRNQVDSETHCHAERCELS
WFCDDILSPNTNYQVTWYTSWSPCPECAGEVAEFLARHSNVNLTIFTARLYYFWDTDYQEGLRS
LNQEGASVKIMGYKDFKYCWENFVYNDDEPFKPWKGLKYNFLFLDSKLQEILE
SEQ ID NO: 33: Uniprot Q1G0Z6
MQPQYRNTVERMYRGTFFYNFNNRPILSRRNTVWLCYEVKTRGPSMPTWDTKIFRGQVYSKPEH
HAEMCFLSRFCGNQLPAYKRFQITWFVSWTPCPDCVAKVAEFLAEHPNVTLTISAARLYYYWET
DYRRALCRLRQAGARVKIMDYEEFAYCWENFVYNEGQSFMPWDKEDDNYAFLHHKLKEILRNPM
EATYPHIFYFHFKNLRKAYGRNETWLCFTMEIIKQHSTVSWETGVFRNQVDPESRCHAERCELS
WFCEDILSPNTDYQVTWYTSWSPCLDCAGEVAEFLARHSNVKLAIFAARLYYFWDTHYQQGLRS
LSEKGASVEIMGYKDFKYCWENFVYNGDEPFKPWKGLKYNFLFLDSKLQEILE
SEQ ID NO: 34: UniProt Q9HC16
MKPHFRNTVERMYRDTFSYNFYNRPILSRRNTVWLCYEVKTKGPSRPPLDAKIFRGQVYSELKY
HPEMRFFHWFSKWRKLHRDQEYEVTWYISWSPCTKCTRDMATFLAEDPKVTLTIFVARLYYFWD
PDYQEALRSLCQKRDGPRATMKIMNYDEFQHCWSKFVYSQRELFEPWNNLPKYYILLHIMLGEI
LRHSMDPPTFTFNENNEPWVRGRHETYLCYEVERMHNDTWVLLNQRRGFLCNQAPHKHGFLEGR
HAELCFLDVIPFWKLDLDQDYRVTCFTSWSPCFSCAQEMAKFISKNKHVSLCIFTARIYDDQGR
CQEGLRTLAEAGAKISIMTYSEFKHCWDTFVDHQGCPFQPWDGLDEHSQDLSGRLRAILQNQEN
SEQ ID NO: 35: Uniprot Q694C1
MTPQFRNTVERMYRDTFSYNFNNRPILSRRNTVWLCYEVKTKDPSRPPLDAKIFRGQVYSELKY
HPEMRFFHWFSKWRKLHRDQEYEVTWYISWSPCTKCTRNVATFLAEDPKVTLTIFVARLYYFWD
QDYQEALRSLCQKRDGPRATMKIMNYDEFQHCWSKFVYSQRELFEPWNNLPKYYMLLHIMLGEI
LRHSMDPPTFTSNFNNEHWVRGRHETYLCYEVERLHNDTWVLLNQRRGFLCNQAPHKHGFLEGR
HAELCFLDVIPFWKLDLHQDYRVTCFTSWSPCFSCAQEMAKFISNKKHVSLCIFAARIYDDQGR
CQEGLRTLAEAGAKISIMTYSEFKHCWDTFVYHQGCPFQPWDGLEEHSQALSGRLQAILQNQGN
SEQ ID NO: 36: Uniprot U5NDB3
MNPQIRNMVEPMDPRTFVSNFNNRPILSGLNTVWLCCEVKTKDPSGPPLDAKIFQGKVLRSKAK
YHPEMRFLQWFREWRQLHHDQEYKVTWYVSWSPCTRCANSVATFLAKDPKVTLTIFVARLYYFW
KPNYQQALRILCQKRDGPHATMKIMNYNEFQDCWNKFVDGRGKPFKPWNNLPKHYTLLQATLGE
LLRHLMDPGTFTSNFNNKPWVSGQHETYLCYKVERLHNDTWVPLNQHRGFLRNQAPNIHGFPKG
RHAELCFLDLIPFWKLDGQQYRVTCFTSWSPCFSCAQEMAKFISNNEHVSLCIFAARIYDDQGR
YQEGLRTLHRDGAKIAMMNYSEFEYCWDTFVDCQGCPFQPWDGLDEHSQALSERLRAILQNQGN
SEQ ID NO: 37: UniProt Q6NTF7
MALLTAETFRLQFNNKRRLRRPYYPRKALLCYQLTPQNGSTPTRGYFENKKKCHAEICFINEIK
SMGLDETQCYQVTCYLTWSPCSSCAWELVDFIKAHDHLNLGIFASRLYYHWCKPQQKGLRLLCG
SQVPVEVMGFPEFADCWENFVDHEKPLSFNPYKMLEELDKNSRAIKRRLERIKIPGVRAQGRYM
DILCDAEV
SEQ ID NO: 38: Uniprot B7T0U7
MALLTAETFRLQFNNKLRLRRPYYRRKTLLCYQLTPQNGSMPTRGYFKNKKKCHAEICFINEIK
SMGLDETQCYQVTCYLTWSPCSSCAWKLVDFIKAHDHLNLRIFASRLYYHWCKRQQEGLRLLCG
SQVPVEVMGFPEFADCWENFVDHEKPLSFDPSKMLEELDKNSQAIKRRLERIKSRSVDVLENGL
RSLQLGPVTPSSSRSNSR
SEQ ID NO: 39: Uniprot Q19Q52
MALLTAKTFSLQFNNKRRVNKPYYPRKALLCYQLTPQNGSTPTRGHLKNKKKDHAEIRFINKIK
SMGLDETQCYQVTCYLTWSPCPSCAGELVDFIKAHRHLNLRIFASRLYYHWRPNYQEGLLLLCG
SQVPVEVMGLPEFTDCWENFVDHKEPPSFNPSEKLEELDKNSQAIKRRLERIKSRSVDVLENGL
RSLQLGPVTPSSSIRNSR
SEQ ID NO: 40: UniProt Q8WW27
MEPIYEEYLANHGTIVKPYYWLSFSLDCSNCPYHIRTGEEARVSLTEFCQIFGFPYGTTFPQTK
HLTFYELKTSSGSLVQKGHASSCTGNYIHPESMLFEMNGYLDSAIYNNDSIRHIILYSNNSPCN
EANHCCISKMYNFLITYPGITLSIYFSQLYHTEMDFPASAWNREALRSLASLWPRVVLSPISGG
IWHSVLHSFISGVSGSHVFQPILTGRALADRHNAYEINAITGVKPYFTDVLLQTKRNPNTKAQE
ALESYPLNNAFPGQFFQMPSGQLQPNLPPDLRAPVVFVLVPLRDLPPMHMGQNPNKPRNIVRHL
NMPQMSFQETKDLGRLPTGRSVEIVEITEQFASSKEADEKKKKKGKK
SEQ ID NO: 41: NCBI XP_004028087.1
MEPIYEEYLANHGTIVKPYYWLSFSLDCSNCPYHIRTGEEARVSLTEFCQIFGFPYGTTFPQTK
HLTFYELKTSSGSLVQKGHASSCTGNYIHPESMLFEMNGYLDSAIYNNDSIRHIILYSNNSPCN
EANHCCISKMYNFLITYPGITLSIYFSQLYHTEMDFPASAWNREALRSLASLWPRVVLSPISGG
IWHSVLHSFISGVSGSHVFQPILTGRALADRHNAYEINAITGVKPYFTDVLLQTKRNPNTKAQE
ALESYPLNNAFPGQSFQMPSGQLQPNLPPDVRAPVVFVLVPLRDLPPMHMGQNPNKPRNIVRHL
NMPQMSFQETEDLGRLPTGRSVEIVEITERFASSKEADEKKKKKKGKK
SEQ ID NO: 42: Uniprot Q497M3
MEPLYEEILTQGGTIVKPYYWLSLSLGCTNCPYHIRTGEEARVPYTEFHQTFGFPWSTYPQTKH
LTFYELRSSSKNLIQKGLASNCTGSHNHPEAMLFEKNGYLDAVIFHNSNIRHIILYSNNSPCNE
AKHCCISKMYNFLMNYPEVTLSVFFSQLYHTEKQFPTSAWNRKALQSLASLWPQVTLSPICGGL
WHAILEKFVSNISGSTVPQPFIAGRILADRYNTYEINSIIAAKPYFTDGLLSRQKENQNREAWA
AFEKHPLGSAAPAQRQPTRGQDPRTPAVLMLVSNRDLPPIHVGSTPQKPRTVVRHLNMLQLSSF
KVKDVKKPPSGRPVEEVEVMKESARSQKANKKNRSQWKKQTLVIKPRICRLLER
SEQ ID NO: 43: UniProt Q8NFU7
MSRSRHARPSRLVRKEDVNKKKKNSQLRKTTKGANKNVASVKTLSPGKLKQLIQERDVKKKTEP
KPPVPVRSLLTRAGAARMNLDRTEVLFQNPESLTCNGFTMALRSTSLSRRLSQPPLVVAKSKKV
PLSKGLEKQHDCDYKILPALGVKHSENDSVPMQDTQVLPDIETLIGVQNPSLLKGKSQETTQFW
SQRVEDSKINIPTHSGPAAEILPGPLEGTRCGEGLFSEETLNDTSGSPKMFAQDTVCAPFPQRA
TPKVTSQGNPSIQLEELGSRVESLKLSDSYLDPIKSEHDCYPTSSLNKVIPDLNLRNCLALGGS
TSPTSVIKFLLAGSKQATLGAKPDHQEAFEATANQQEVSDTTSFLGQAFGAIPHQWELPGADPV
HGEALGETPDLPEIPGAIPVQGEVFGTILDQQETLGMSGSVVPDLPVFLPVPPNPIATFNAPSK
WPEPQSTVSYGLAVQGAIQILPLGSGHTPQSSSNSEKNSLPPVMAISNVENEKQVHISFLPANT
QGFPLAPERGLFHASLGIAQLSQAGPSKSDRGSSQVSVTSTVHVVNTTVVTMPVPMVSTSSSSY
TTLLPTLEKKKRKRCGVCEPCQQKTNCGECTYCKNRKNSHQICKKRKCEELKKKPSVVVPLEVI
KENKRPQREKKPKVLKADFDNKPVNGPKSESMDYSRCGHGEEQKLELNPHTVENVTKNEDSMTG
IEVEKWTQNKKSQLTDHVKGDFSANVPEAEKSKNSEVDKKRTKSPKLFVQTVRNGIKHVHCLPA
ETNVSFKKFNIEEFGKTLENNSYKFLKDTANHKNAMSSVATDMSCDHLKGRSNVLVFQQPGENC
SSIPHSSHSIINHHASIHNEGDQPKTPENIPSKEPKDGSPVQPSLLSLMKDRRLTLEQVVAIEA
LTQLSEAPSENSSPSKSEKDEESEQRTASLLNSCKAILYTVRKDLQDPNLQGEPPKINHCPSLE
KQSSCNTVVFNGQTTTLSNSHINSATNQASTKSHEYSKVINSLSLFIPKSNSSKIDTNKSIAQG
IITLDNCSNDLHQLPPRNNEVEYCNQLLDSSKKLDSDDLSCQDATHTQIEEDVATQLTQLASII
KINYIKPEDKKVESTPTSLVTCNVQQKYNQEKGTIQQKPPSSVHNNHGSSLTKQKNPTQKKTKS
TPSRDRRKKKPTVVSYQENDRQKWEKLSYMYGTICDIWIASKFQNFGQFCPHDFPTVFGKISSS
TKIWKPLAQTRSIMQPKTVFPPLTQIKLQRYPESAEEKVKVEPLDSLSLFHLKTESNGKAFTDK
AYNSQVQLTVNANQKAHPLTQPSSPPNQCANVMAGDDQIRFQQVVKEQLMHQRLPTLPGISHET
PLPESALTLRNVNVVCSGGITVVSTKSEEEVCSSSFGTSEFSTVDSAQKNFNDYAMNFFTNPTK
NLVSITKDSELPTCSCLDRVIQKDKGPYYTHLGAGPSVAAVREIMENRYGQKGNAIRIEIVVYT
GKEGKSSHGCPIAKWVLRRSSDEEKVLCLVRQRTGHHCPTAVMVVLIMVWDGIPLPMADRLYTE
LTENLKSYNGHPTDRRCTLNENRTCTCQGIDPETCGASFSFGCSWSMYFNGCKFGRSPSPRRFR
IDPSSPLHEKNLEDNLQSLATRLAPIYKQYAPVAYQNQVEYENVARECRLGSKEGRPFSGVTAC
LDFCAHPHRDIHNMNNGSTVVCTLTREDNRSLGVIPQDEQLHVLPLYKLSDTDEFGSKEGMEAK
IKSGAIEVLAPRRKKRTCFTQPVPRSGKKRAAMMTEVLAHKIRAVEKKPIPRIKRKNNSTTTNN
SKPSSLPTLGSNTETVQPEVKSETEPHFILKSSDNTKTYSLMPSAPHPVKEASPGFSWSPKTAS
ATPAPLKNDATASCGFSERSSTPHCTMPSGRLSGANAAAADGPGISQLGEVAPLPTLSAPVMEP
LINSEPSTGVTEPLTPHQPNHQPSFLTSPQDLASSPMEEDEQHSEADEPPSDEPLSDDPLSPAE
EKLPHIDEYWSDSEHIFLDANIGGVAIAPAHGSVLIECARRELHATTPVEHPNRNHPTRLSLVE
YQHKNLNKPQHGFELNKIKFEAKEAKNKKMKASEQKDQAANEGPEQSSEVNELNQIPSHKALTL
THDNVVTVSPYALTHVAGPYNHWV
SEQ ID NO: 44: UniProt Q3URK3
MSRSRPAKPSKSVKTKLQKKKDIQMKTKTSKQAVRHGASAKAVNPGKPKQLIKRRDGKKETEDK
TPTPAPSFLTRAGAARMNRDRNQVLFQNPDSLTCNGFTMALRRTSLSWRLSQRPVVTPKPKKVP
PSKKQCTHNIQDEPGVKHSENDSVPSQHATVSPGTENGEQNRCLVEGESQEITQSCPVFEERIE
DTQSCISASGNLEAEISWPLEGTHCEELLSHQTSDNECTSPQECAPLPQRSTSEVTSQKNTSNQ
LADLSSQVESIKLSDPSPNPTGSDHNGFPDSSFRIVPELDLKTCMPLDESVYPTALIRFILAGS
QPDVFDTKPQEKTLITTPEQVGSHPNQVLDATSVLGQAFSTLPLQWGFSGANLVQVEALGKGSD
SPEDLGAITMLNQQETVAMDMDRNATPDLPIFLPKPPNTVATYSSPLLGPEPHSSTSCGLEVQG
ATPILTLDSGHTPQLPPNPESSSVPLVIAANGTRAEKQFGTSLFPAVPQGFTVAAENEVQHAPL
DLTQGSQAAPSKLEGEISRVSITGSADVKATAMSMPVTQASTSSPPCNSTPPMVERRKRKACGV
CEPCQQKANCGECTYCKNRKNSHQICKKRKCEVLKKKPEATSQAQVTKENKRPQREKKPKVLKT
DFNNKPVNGPKSESMDCSRRGHGEEEQRLDLITHPLENVRKNAGGMTGIEVEKWAPNKKSHLAE
GQVKGSCDANLTGVENPQPSEDDKQQTNPSPTFAQTIRNGMKNVHCLPTDTHLPLNKLNHEEFS
KALGNNSSKLLTDPSNCKDAMSVTTSGGECDHLKGPRNTLLFQKPGLNCRSGAEPTIENNHPNT
HSAGSRPHPPEKVPNKEPKDGSPVQPSLLSLMKDRRLTLEQVVAIEALTQLSEAPSESSSPSKP
EKDEEAHQKTASLLNSCKAILHSVRKDLQDPNVQGKGLHHDTVVENGQNRTFKSPDSFATNQAL
IKSQGYPSSPTAEKKGAAGGRAPFDGFENSHPLPIESHNLENCSQVLSCDQNLSSHDPSCQDAP
YSQIEEDVAAQLTQLASTINHINAEVRNAESTPESLVAKNTKQKHSQEKRMVHQKPPSSTQTKP
SVPSAKPKKAQKKARATPHANKRKKKPPARSSQENDQKKQEQLAIEYSKMHDIWMSSKFQRFGQ
SSPRSFPVLLRNIPVFNQILKPVTQSKTPSQHNELFPPINQIKFTRNPELAKEKVKVEPSDSLP
TCQFKTESGGQTFAEPADNSQGQPMVSVNQEAHPLPQSPPSNQCANIMAGAAQTQFHLGAQENL
VHQIPPPTLPGTSPDTLLPDPASILRKGKVLHFDGITVVTEKREAQTSSNGPLGPTTDSAQSEF
KESIMDLLSKPAKNLIAGLKEQEAAPCDCDGGTQKEKGPYYTHLGAGPSVAAVRELMETRFGQK
GKAIRIEKIVETGKEGKSSQGCPVAKWVIRRSGPEEKLICLVRERVDHHCSTAVIVVLILLWEG
IPRLMADRLYKELTENLRSYSGHPTDRRCTLNKKRTCTCQGIDPKTCGASFSFGCSWSMYENGC
KFGRSENPRKFRLAPNYPLHEKQLEKNLQELATVLAPLYKQMAPVAYQNQVEYEEVAGDCRLGN
EEGRPFSGVTCCMDFCAHSHKDIHNMHNGSTVVCTLIRADGRDTNCPEDEQLHVLPLYRLADTD
EFGSVEGMKAKIKSGAIQVNGPTRKRRLRFTEPVPRCGKRAKMKQNHNKSGSHNTKSESSASST
SHLVKDESTDFCPLQASSAETSTCTYSKTASGGFAETSSILHCTMPSGAHSGANAAAGECTGTV
QPAEVAAHPHQSLPTADSPVHAEPLTSPSEQLTSNQSNQQLPLLSNSQKLASCQVEDERHPEAD
EPQHPEDDNLPQLDEFWSDSEEIYADPSFGGVAIAPIHGSVLIECARKELHATTSLRSPKRGVP
FRVSLVFYQHKSLNKPNHGFDINKIKCKCKKVTKKKPADRECPDVSPEANLSHQIPSRVASTLT
RDNVVTVSPYSLTHVAGPYNRWV
SEQ ID NO: 45: UniProt Q6N021
MEQDRTNHVEGNRLSPFLIPSPPICQTEPLATKLQNGSPLPERAHPEVNGDTKWHSFKSYYGIP
CMKGSQNSRVSPDFTQESRGYSKCLQNGGIKRTVSEPSLSGLLQIKKLKQDQKANGERRNFGVS
QERNPGESSQPNVSDLSDKKESVSSVAQENAVKDFTSFSTHNCSGPENPELQILNEQEGKSANY
HDKNIVLLKNKAVLMPNGATVSASSVEHTHGELLEKTLSQYYPDCVSIAVQKTTSHINAINSQA
TNELSCEITHPSHTSGQINSAQTSNSELPPKPAAVVSEACDADDADNASKLAAMLNTCSFQKPE
QLQQQKSVFEICPSPAENNIQGTTKLASGEEFCSGSSSNLQAPGGSSERYLKQNEMNGAYFKQS
SVFTKDSFSATTTPPPPSQLLLSPPPPLPQVPQLPSEGKSTLNGGVLEEHHHYPNQSNTTLLRE
VKIEGKPEAPPSQSPNPSTHVCSPSPMLSERPQNNCVNRNDIQTAGTMTVPLCSEKTRPMSEHL
KHNPPIFGSSGELQDNCQQLMRNKEQEILKGRDKEQTRDLVPPTQHYLKPGWIELKAPRFHQAE
SHLKRNEASLPSILQYQPNLSNQMTSKQYTGNSNMPGGLPRQAYTQKTTQLEHKSQMYQVEMNQ
GQSQGTVDQHLQFQKPSHQVHFSKTDHLPKAHVQSLCGTRFHFQQRADSQTEKLMSPVLKQHLN
QQASETEPFSNSHLLQHKPHKQAAQTQPSQSSHLPQNQQQQQKLQIKNKEEILQTFPHPQSNND
QQREGSFFGQTKVEECFHGENQYSKSSEFETHNVQMGLEEVQNINRRNSPYSQTMKSSACKIQV
SCSNNTHLVSENKEQTTHPELFAGNKTQNLHHMQYFPNNVIPKQDLLHRCFQEQEQKSQQASVL
QGYKNRNQDMSGQQAAQLAQQRYLIHNHANVFPVPDQGGSHTQTPPQKDTQKHAALRWHLLQKQ
EQQQTQQPQTESCHSQMHRPIKVEPGCKPHACMHTAPPENKTWKKVTKQENPPASCDNVQQKSI
IETMEQHLKQFHAKSLFDHKALTLKSQKQVKVEMSGPVTVLTRQTTAAELDSHTPALEQQTTSS
EKTPTKRTAASVLNNFIESPSKLLDTPIKNLLDTPVKTQYDFPSCRCVEQIIEKDEGPFYTHLG
AGPNVAAIREIMEERFGQKGKAIRIERVIYTGKEGKSSQGCPIAKWVVRRSSSEEKLLCLVRER
AGHTCEAAVIVILILVWEGIPLSLADKLYSELTETLRKYGTLTNRRCALNEERTCACQGLDPET
CGASFSFGCSWSMYYNGCKFARSKIPRKFKLLGDDPKEEEKLESHLQNLSTLMAPTYKKLAPDA
YNNQIEYEHRAPECRLGLKEGRPFSGVTACLDFCAHAHRDLHNMQNGSTLVCTLTREDNREFGG
KPEDEQLHVLPLYKVSDVDEFGSVEAQEEKKRSGAIQVLSSFRRKVRMLAEPVKTCRQRKLEAK
KAAAEKLSSLENSSNKNEKEKSAPSRTKQTENASQAKQLAELLRLSGPVMQQSQQPQPLQKQPP
QPQQQQRPQQQQPHHPQTESVNSYSASGSTNPYMRRPNPVSPYPNSSHTSDIYGSTSPMNFYST
SSQAAGSYLNSSNPMNPYPGLLNQNTQYPSYQCNGNLSVDNCSPYLGSYSPQSQPMDLYRYPSQ
DPLSKLSLPPIHTLYQPRFGNSQSFTSKYLGYGNQNMQGDGFSSCTIRPNVHHVGKLPPYPTHE
MDGHFMGATSRLPPNLSNPNMDYKNGEHHSPSHIIHNYSAAPGMFNSSLHALHLQNKENDMLSH
TANGLSKMLPALNHDRTACVQGGLHKLSDANGQEKQPLALVQGVASGAEDNDEVWSDSEQSFLD
PDIGGVAVAPTHGSILIECAKRELHATTPLKNPNRNHPTRISLVFYQHKSMNEPKHGLALWEAK
MAEKAREKEEECEKYGPDYVPQKSHGKKVKREPAEPHETSEPTYLRFIKSLAERTMSVTTDSTV
TTSPYAFTRVTGPYNRYI
SEQ ID NO: 46: UniProt Q4JK59
MEQDRTTHAEGTRLSPFLIAPPSPISHTEPLAVKLQNGSPLAERPHPEVNGDTKWQSSQSCYGI
SHMKGSQSSHESPHEDRGYSRCLQNGGIKRTVSEPSLSGLHPNKILKLDQKAKGESNIFEESQE
RNHGKSSRQPNVSGLSDNGEPVTSTTQESSGADAFPTRNYNGVEIQVLNEQEGEKGRSVTLLKN
KIVLMPNGATVSAHSEENTRGELLEKTQCYPDCVSIAVQSTASHVNTPSSQAAIELSHEIPQPS
LTSAQINFSQTSSLQLPPEPAAMVTKACDADNASKPAIVPGTCPFQKAEHQQKSALDIGPSRAE
NKTIQGSMELFAEEYYPSSDRNLQASHGSSEQYSKQKETNGAYFRQSSKFPKDSISPTTVTPPS
QSLLAPRLVLQPPLEGKGALNDVALEEHHDYPNRSNRTLLREGKIDHQPKTSSSQSLNPSVHTP
NPPLMLPEQHQNDCGSPSPEKSRKMSEYLMYYLPNHGHSGGLQEHSQYLMGHREQEIPKDANGK
QTQGSVQAAPGWIELKAPNLHEALHQTKRKDISLHSVLHSQTGPVNQMSSKQSTGNVNMPGGFQ
RLPYLQKTAQPEQKAQMYQVQVNQGPSPGMGDQHLQFQKALYQECIPRTDPSSEAHPQAPSVPQ
YHFQQRVNPSSDKHLSQQATETQRLSGFLQHTPQTQASQTPASQNSNFPQICQQQQQQQLQRKN
KEQMPQTFSHLQGSNDKQREGSCFGQIKVEESFCVGNQYSKSSNFQTHNNTQGGLEQVQNINKN
FPYSKILTPNSSNLQILPSNDTHPACEREQALHPVGSKTSNLQNMQYFPNNVTPNQDVHRCFQE
QAQKPQQASSLQGLKDRSQGESPAPPAEAAQQRYLVHNEAKALPVPEQGGSQTQTPPQKDTQKH
AALRWLLLQKQEQQQTQQSQPGHNQMLRPIKTEPVSKPSSYRYPLSPPQENMSSRIKQEISSPS
RDNGQPKSIIETMEQHLKQFQLKSLCDYKALTLKSQKHVKVPTDIQAAESENHARAAEPQATKS
TDCSVLDDVSESDTPGEQSQNGKCEGCNPDKDEAPYYTHLGAGPDVAAIRTLMEERYGEKGKAI
RIEKVIYTGKEGKSSQGCPIAKWVYRRSSEEEKLLCLVRVRPNHTCETAVMVIAIMLWDGIPKL
LASELYSELTDILGKCGICTNRRCSQNETRNCCCQGENPETCGASFSFGCSWSMYYNGCKFARS
KKPRKFRLHGAEPKEEERLGSHLQNLATVIAPIYKKLAPDAYNNQVEFEHQAPDCCLGLKEGRP
FSGVTACLDFSAHSHRDQQNMPNGSTVVVTLNREDNREVGAKPEDEQFHVLPMYIIAPEDEFGS
TEGQEKKIRMGSIEVLQSFRRRRVIRIGELPKSCKKKAEPKKAKTKKAARKRSSLENCSSRTEK
GKSSSHTKLMENASHMKQMTAQPQLSGPVIRQPPTLQRHLQQGQRPQQPQPPQPQPQTTPQPQP
QPQHIMPGNSQSVGSHCSGSTSVYTRQPTPHSPYPSSAHTSDIYGDTNHVNFYPTSSHASGSYL
NPSNYMNPYLGLLNQNNQYAPFPYNGSVPVDNGSPFLGSYSPQAQSRDLHRYPNQDHLTNQNLP
PIHTLHQQTFGDSPSKYLSYGNQNMQRDAFTTNSTLKPNVHHLATFSPYPTPKMDSHFMGAASR
SPYSHPHTDYKTSEHHLPSHTIYSYTAAASGSSSSHAFHNKENDNIANGLSRVLPGENHDRTAS
AQELLYSLTGSSQEKQPEVSGQDAAAVQEIEYWSDSEHNFQDPCIGGVAIAPTHGSILIECAKC
EVHATTKVNDPDRNHPTRISLVLYRHKNLFLPKHCLALWEAKMAEKARKEEECGKNGSDHVSQK
NHGKQEKREPTGPQEPSYLRFIQSLAENTGSVTTDSTVTTSPYAFTQVTGPYNTFV
SEQ ID NO: 47: UniProt O43151
MSQFQVPLAVQPDLPGLYDFPQRQVMVGSFPGSGLSMAGSESQLRGGGDGRKKRKRCGTCEPCR
RLENCGACTSCTNRRTHQICKLRKCEVLKKKVGLLKEVEIKAGEGAGPWGQGAAVKTGSELSPV
DGPVPGQMDSGPVYHGDSRQLSASGVPVNGAREPAGPSLLGTGGPWRVDQKPDWEAAPGPAHTA
RLEDAHDLVAFSAVAEAVSSYGALSTRLYETFNREMSREAGNNSRGPRPGPEGCSAGSEDLDTL
QTALALARHGMKPPNCNCDGPECPDYLEWLEGKIKSVVMEGGEERPRLPGPLPPGEAGLPAPST
RPLLSSEVPQISPQEGLPLSQSALSIAKEKNISLQTAIAIEALTQLSSALPQPSHSTPQASCPL
PEALSPPAPERSPQSYLRAPSWPVVPPEEHSSFAPDSSAFPPATPRTEFPEAWGTDTPPATPRS
SWPMPRPSPDPMAELEQLLGSASDYIQSVFKRPEALPTKPKVKVEAPSSSPAPAPSPVLQREAP
TPSSEPDTHQKAQTALQQHLHHKRSLFLEQVHDTSFPAPSEPSAPGWWPPPSSPVPRLPDRPPK
EKKKKLPTPAGGPVGTEKAAPGIKPSVRKPIQIKKSRPREAQPLFPPVRQIVLEGLRSPASQEV
QAHPPAPLPASQGSAVPLPPEPSLALFAPSPSRDSLLPPTQEMRSPSPMTALQPGSTGPLPPAD
DKLEELIRQFEAEFGDSFGLPGPPSVPIQDPENQQTCLPAPESPFATRSPKQIKIESSGAVTVL
STTCFHSEEGGQEATPTKAENPLTPTLSGFLESPLKYLDTPTKSLLDTPAKRAQAEFPTCDCVE
QIVEKDEGPYYTHLGSGPTVASIRELMEERYGEKGKAIRIEKVIYTGKEGKSSRGCPIAKWVIR
RHTLEEKLLCLVRHRAGHHCQNAVIVILILAWEGIPRSLGDTLYQELTDTLRKYGNPTSRRCGL
NDDRTCACQGKDPNTCGASFSFGCSWSMYFNGCKYARSKTPRKFRLAGDNPKEEEVLRKSFQDL
ATEVAPLYKRLAPQAYQNQVTNEEIAIDCRLGLKEGRPFAGVTACMDFCAHAHKDQHNLYNGCT
VVCTLTKEDNRCVGKIPEDEQLHVLPLYKMANTDEFGSEENQNAKVGSGAIQVLTAFPREVRRL
PEPAKSCRQRQLEARKAAAEKKKIQKEKLSTPEKIKQEALELAGITSDPGLSLKGGLSQQGLKP
SLKVEPQNHFSSFKYSGNAVVESYSVLGNCRPSDPYSMNSVYSYHSYYAQPSLTSVNGFHSKYA
LPSFSYYGFPSSNPVFPSQFLGPGAWGHSGSSGSFEKKPDLHALHNSLSPAYGGAEFAELPSQA
VPTDAHHPTPHHQQPAYPGPKEYLLPKAPLLHSVSRDPSPFAQSSNCYNRSIKQEPVDPLTQAE
PVPRDAGKMGKTPLSEVSQNGGPSHLWGQYSGGPSMSPKRTNGVGGSWGVFSSGESPAIVPDKL
SSFGASCLAPSHFTDGQWGLFPGEGQQAASHSGGRLRGKPWSPCKFGNSTSALAGPSLTEKPWA
LGAGDFNSALKGSPGFQDKLWNPMKGEEGRIPAAGASQLDRAWQSFGLPLGSSEKLFGALKSEE
KLWDPFSLEEGPAEEPPSKGAVKEEKGGGGAEEEEEELWSDSEHNFLDENIGGVAVAPAHGSIL
IECARRELHATTPLKKPNRCHPTRISLVFYQHKNLNQPNHGLALWEAKMKQLAERARARQEEAA
RLGLGQQEAKLYGKKRKWGGTVVAEPQQKEKKGVVPTRQALAVPTDSAVTVSSYAYTKVTGPYS
RWI
SEQ ID NO: 48: UniProt Q8BG87
MSQFQVPLAVQPDLSGLYDFPQGQVMVGGFQGPGLPMAGSETQLRGGGDGRKKRKRCGTCDPCR
RLENCGSCTSCTNRRTHQICKLRKCEVLKKKAGLLKEVEINAREGTGPWAQGATVKTGSELSPV
DGPVPGQMDSGPVYHGDSRQLSTSGAPVNGAREPAGPGLLGAAGPWRVDQKPDWEAASGPTHAA
RLEDAHDLVAFSAVAEAVSSYGALSTRLYETENREMSREAGSNGRGPRPESCSEGSEDLDTLQT
ALALARHGMKPPNCTCDGPECPDFLEWLEGKIKSMAMEGGQGRPRLPGALPPSEAGLPAPSTRP
PLLSSEVPQVPPLEGLPLSQSALSIAKEKNISLQTAIAIEALTQLSSALPQPSHSTSQASCPLP
EALSPSAPFRSPQSYLRAPSWPVVPPEEHPSFAPDSPAFPPATPRPEFSEAWGTDTPPATPRNS
WPVPRPSPDPMAELEQLLGSASDYIQSVFKRPEALPTKPKVKVEAPSSSPAPVPSPISQREAPL
LSSEPDTHQKAQTALQQHLHHKRNLFLEQAQDASFPTSTEPQAPGWWAPPGSPAPRPPDKPPKE
KKKKPPTPAGGPVGAEKTTPGIKTSVRKPIQIKKSRSRDMQPLFLPVRQIVLEGLKPQASEGQA
PLPAQLSVPPPASQGAASQSCATPLTPEPSLALFAPSPSGDSLLPPTQEMRSPSPMVALQSGST
GGPLPPADDKLEELIRQFEAEFGDSFGLPGPPSVPIQEPENQSTCLPAPESPFATRSPKKIKIE
SSGAVTVLSTTCFHSEEGGQEATPTKAENPLTPTLSGFLESPLKYLDTPTKSLLDTPAKKAQSE
FPTCDCVEQIVEKDEGPYYTHLGSGPTVASIRELMEDRYGEKGKAIRIEKVIYTGKEGKSSRGC
PIAKWVIRRHTLEEKLLCLVRHRAGHHCQNAVIVILILAWEGIPRSLGDTLYQELTDTLRKYGN
PTSRRCGLNDDRTCACQGKDPNTCGASFSFGCSWSMYFNGCKYARSKTPRKFRLTGDNPKEEEV
LRNSFQDLATEVAPLYKRLAPQAYQNQVTNEDVAIDCRLGLKEGRPFSGVTACMDFCAHAHKDQ
HNLYNGCTVVCTLTKEDNRCVGQIPEDEQLHVLPLYKMASTDEFGSEENQNAKVSSGAIQVLTA
FPREVRRLPEPAKSCRQRQLEARKAAAEKKKLQKEKLSTPEKIKQEALELAGVTTDPGLSLKGG
LSQQSLKPSLKVEPQNHFSSFKYSGNAVVESYSVLGSCRPSDPYSMSSVYSYHSRYAQPGLASV
NGFHSKYTLPSFGYYGFPSSNPVFPSQFLGPSAWGHGGSGGSFEKKPDLHALHNSLNPAYGGAE
FAELPGQAVATDNHHPIPHHQQPAYPGPKEYLLPKVPQLHPASRDPSPFAQSSSCYNRSIKQEP
IDPLTQAESIPRDSAKMSRTPLPEASQNGGPSHLWGQYSGGPSMSPKRTNSVGGNWGVFPPGES
PTIVPDKLNSFGASCLTPSHFPESQWGLFTGEGQQSAPHAGARLRGKPWSPCKFGNGTSALTGP
SLTEKPWGMGTGDFNPALKGGPGFQDKLWNPVKVEEGRIPTPGANPLDKAWQAFGMPLSSNEKL
FGALKSEEKLWDPFSLEEGTAEEPPSKGVVKEEKSGPTVEEDEEELWSDSEHNFLDENIGGVAV
APAHCSILIECARRELHATTPLKKPNRCHPTRISLVFYQHKNLNQPNHGLALWEAKMKQLAERA
RQRQEEAARLGLGQQEAKLYGKKRKWGGAMVAEPQHKEKKGAIPTRQALAMPTDSAVTVSSYAY
TKVTGPYSRWI
SEQ ID NO: 49: UniProt P04519
MRICIFMARGLEGCGVTKFSLEQRDWFIKNGHEVTLVYAKDKSFTRTSSHDHKSFSIPVILAKE
YDKALKLVNDCDILIINSVPATSVQEATINNYKKLLDNIKPSIRVVVYQHDHSVLSLRRNLGLE
ETVRRADVIFSHSDNGDFNKVLMKEWYPETVSLFDDIEEAPTVYNFQPPMDIVKVRSTYWKDVS
EINMNINRWIGRTTTWKGFYQMFDFHEKFLKPAGKSTVMEGLERSPAFIAIKEKGIPYEYYGNR
EIDKMNLAPNQPAQILDCYINSEMLERMSKSGFGYQLSKLNQKYLQRSLEYTHLELGACGTIPV
FWKSTGENLKFRVDNTPLTSHDSGIIWFDENDMESTFERIKELSSDRALYDREREKAYEFLYQH
QDSSFCFKEQFDIITK
SEQ ID NO: 50: UniProt P04547
MKIAIINMGNNVINFKTVPSSETIYLFKVISEMGLNVDIISLKNGVYTKSFDEVDVNDYDRLIV
VNSSINFFGGKPNLAILSAQKFMAKYKSKIYYLFTDIRLPFSQSWPNVKNRPWAYLYTEEELLI
KSPIKVISQGINLDIAKAAHKKVDNVIEFEYFPIEQYKIHMNDFQLSKPTKKTLDVIYGGSFRS
GQRESKMVEFLFDTGLNIEFFGNAREKQFKNPKYPWTKAPVFTGKIPMNMVSEKNSQAIAALII
GDKNYNDNFITLRVWETMASDAVMLIDEEFDTKHRIINDARFYVNNRAELIDRVNELKHSDVLR
KEMLSIQHDILNKTRAKKAEWQDAFKKAIDL
SEQ ID NO: 51: ZDD motif
H-[P/A/V]-E-X_{[23-28]}-P-C-X_{[2-4]}-C
SEQ ID NO: 52: ZDD motif
HXEX₂₄SW(S/T)PCX_{[2-4]}CX₆FX₈LX₅R(L/I)YX_{[8-11]}LX₂LX_{[10]}M
SEQ ID NO. 53: Removable P5 sequence
TTTTTTTTTTAATGATACGGCGACCACCGAUCTACAC
(where U = 2-deoxyuridine)
SEQ ID NO. 54: Removable P7 sequence
TTTTTTTTTTCAAGCAGAAGACGGCATACGA[G^{OXO}]AT
(where [G^{OXO}] = 8-oxoguanine)
SEQ ID NO: 55: Extended primer sequence with A as 5′ additional nucleotide and P5′
sequence (complementary to P5)
AGTGTAGATCTCGGTGGTCGCCGTATCATT
SEQ ID NO: 56: Extended primer sequence with T as 5′ additional nucleotide and P5′
sequence (complementary to P5)
TGTGTAGATCTCGGTGGTCGCCGTATCATT
SEQ ID NO: 57: Extended primer sequence with C as 5′ additional nucleotide and P5′
sequence (complementary to P5)
CGTGTAGATCTCGGTGGTCGCCGTATCATT
SEQ ID NO: 58: Extended primer sequence with G as 5′ additional nucleotide and P5′
sequence (complementary to P5)
GGTGTAGATCTCGGTGGTCGCCGTATCATT
SEQ ID NO: 59: Extended primer sequence with A as 5′ additional nucleotide and P7′
sequence (complementary to P7)
AATCTCGTATGCCGTCTTCTGCTTG
SEQ ID NO: 60: Extended primer sequence with T as 5′ additional nucleotide and P7′
sequence (complementary to P7)
TATCTCGTATGCCGTCTTCTGCTTG
SEQ ID NO: 61: Extended primer sequence with C as 5′ additional nucleotide and P7′
sequence (complementary to P7)
CATCTCGTATGCCGTCTTCTGCTTG
SEQ ID NO: 62: Extended primer sequence with G as 5′ additional nucleotide and P7′
sequence (complementary to P7)
GATCTCGTATGCCGTCTTCTGCTTG
SEQ ID NO: 63: Extended primer sequence with A as 5′ additional nucleotide and
alternative P5′ sequence (complementary to alternative P5)
ATCGGTCGCCGTATCATT
SEQ ID NO: 64: Extended primer sequence with T as 5′ additional nucleotide and
alternative P5′ sequence (complementary to alternative P5)
TTCGGTCGCCGTATCATT
SEQ ID NO: 65: Extended primer sequence with C as 5′ additional nucleotide and
alternative P5′ sequence (complementary to alternative P5)
CTCGGTCGCCGTATCATT
SEQ ID NO: 66: Extended primer sequence with G as 5′ additional nucleotide and
alternative P5′ sequence (complementary to alternative P5)
GTCGGTCGCCGTATCATT
SEQ ID NO: 67: P5_BbvCI_P7
GCTGAGGATCTCGTATGCCGTCTTCTGCTTGUAATGATACGGCGACCACCGAGATCTACACTCC
TCAGC*T
(where asterisk (*) indicates phosphorothioate linkage)
SEQ ID NO: 68: BspQI_iSce_Loop
GAAGAGCACACGTCTGAACTCCAGTCACTAGGGA[Biotin-T]AACAGGGTAATCTTTCCCTA
CACGACGCTCTTC*T
(where asterisk (*) indicates phosphorothioate linkage, [Biotin-T] is a modified thymine
residue comprising biotin)
SEQ ID NO: 69: P5_BbvCI_P7-methylated
GCTGAGGATCTCGTATGCCGTCTTCTGCTTGUAATGATACGGCGACCACCGAGATCTACACTCC
TCAGC*T
(where asterisk (*) indicates phosphorothioate linkage, underline indicates 5-
methylcytosine (i.e. all cytosines are methylated))
SEQ ID NO: 70: BspQI_iSce_Loop-methylated
GAAGAGCACACGTCTGAACTCCAGTCACTAGGGA[Biotin-T]AACAGGGTAATCTTTCCCTA
CACGACGCTCTTC*T
(where asterisk (*) indicates phosphorothioate linkage, [Biotin-T] is a modified thymine
residue comprising biotin, underline indicates 5-methylcytosine (i.e. all cytosines are
methylated))

1. A method of preparing polynucleotide sequences for detection of modified cytosines, comprising:
synthesising at least one first polynucleotide sequence comprising a first portion and at least one second polynucleotide sequence comprising a second portion,
wherein the at least one first polynucleotide sequence comprising a first portion and the at least one second polynucleotide sequence comprising a second portion each comprise portions of a double-stranded nucleic acid template, and the first portion comprises a forward strand of the template, and the second portion comprises a reverse complement strand of the template; or wherein the first portion comprises a reverse strand of the template, and the second portion comprises a forward complement strand of the template,
wherein the template is generated from a target polynucleotide to be sequenced via complementary base pairing, and wherein the target polynucleotide has been pre-treated using a conversion reagent,
wherein the conversion reagent is configured to convert a modified cytosine to thymine or a nucleobase which is read as thymine/uracil, and/or wherein the conversion reagent is configured to convert an unmodified cytosine to uracil or a nucleobase which is read as thymine/uracil.

2. The method according to claim 1, wherein the target polynucleotide has been pre-treated using a conversion reagent configured to convert a modified cytosine to thymine or a nucleobase which is read as thymine/uracil.

3. The method according to claim 1, wherein the target polynucleotide has been pre-treated using a conversion reagent configured to convert an unmodified cytosine to uracil or a nucleobase which is read as thymine/uracil.

4. The method according to claim 1, wherein the conversion agent comprises a chemical agent and/or an enzyme.

5.-18. (canceled)

19. The method according to claim 1, wherein the target polynucleotide is treated with a further agent prior to treatment with the conversion reagent.

20.-28. (canceled)

29. The method according to claim 1, wherein the modified cytosine is selected from the group consisting of: 5-methylcytosine, 5-hydroxymethylcytosine, 5-formylcytosine and 5-carboxylcytosine.

30. The method according to claim 1, wherein the forward strand of the template is not identical to the reverse complement strand of the template.

31. (canceled)

32. The method according to claim 1, wherein the method further comprises a step of preparing the first portion and the second portion for concurrent sequencing.

33. (canceled)

34. (canceled)

35. The method according to claim 1, wherein a proportion of first portions is capable of generating a first signal and a proportion of second portions is capable of generating a second signal, wherein an intensity of the first signal is substantially the same as an intensity of the second signal.

36. The method according to claim 1, wherein the method further comprises a step of selectively processing the at least one first polynucleotide sequence comprising a first portion and the at least one second polynucleotide sequence comprising a second portion, such that a proportion of first portions are capable of generating a first signal and a proportion of second portions are capable of generating a second signal, wherein the selective processing causes an intensity of the first signal to be greater than an intensity of the second signal.

37.-51. (canceled)

52. The method according to claim 1, wherein the first signal and the second signal are spatially unresolved.

53. The method according to claim 1, wherein the at least one first polynucleotide sequence comprising the first portion and the at least one second polynucleotide sequence comprising the second portion are attached to a solid support, preferably wherein the solid support is a flow cell.

54.-60. (canceled)

61. The method according to claim 1, wherein the step of synthesising at least one first polynucleotide sequence comprising a first portion and at least one second polynucleotide sequence comprising a second portion comprises:
synthesising a loop-ligated precursor polynucleotide by connecting a 3′-end of the forward strand of the target polynucleotide and a 5′-end of the reverse strand of the target polynucleotide with a loop, or connecting a 5′-end of the forward strand of the target polynucleotide and a 3′-end of the reverse strand of the target polynucleotide with a loop,
synthesising the at least one first polynucleotide sequence comprising the first portion by forming a complement of the loop-ligated precursor polynucleotide,
synthesising the at least one second polynucleotide sequence comprising the at least one second polynucleotide sequence by forming a complement of the at least one first polynucleotide sequence.

62. The method according to claim 1, wherein the method further comprises concurrently sequencing nucleobases in the first portion and the second portion.

63. A method of sequencing polynucleotide sequences to detect modified cytosines, comprising:
preparing polynucleotide sequences for detection of modified cytosines using a method according to claim 1;
concurrently sequencing nucleobases in the first portion and the second portion; and
identifying modified cytosines by detecting differences when comparing a sequence output from the first portion with a sequence output from the second portion.

64. The method according to claim 63, wherein the step of concurrently sequencing nucleobases comprises performing sequencing-by-synthesis or sequencing-by-ligation.

65. The method according to claim 63, wherein the step of preparing the polynucleotide sequences comprises using a method according to any one of claims 35 to 52; and wherein the step of concurrent sequencing nucleobases in the first portion and the second portion is based on the intensity of the first signal and the intensity of the second signal.

66. The method according to claim 63, wherein the method further comprises a step of conducting paired-end reads.

67. A kit comprising instructions for preparing polynucleotide sequences for detection of modified cytosines according to claim 1, and/or for sequencing polynucleotide sequences to detect modified cytosines according to claim 63.

68. (canceled)

69. (canceled)

70. A computer program product comprising instructions which, when the program is executed by a processor, cause the processor to carry out a method according to claim 1.

71. (canceled)

72. (canceled)

73. (canceled)


SEQUENCE LISTING (70 sequences)

SEQ ID NO: 1 - DNA, 29 bp, unidentified
  source 1..29: mol_type = other DNA
aatgatacggcgaccaccgagatctacac

SEQ ID NO: 2 - DNA, 24 bp, unidentified
  source 1..24: mol_type = unassigned DNA
caagcagaagacggcatacgagat

SEQ ID NO: 3 - DNA, 29 bp, unidentified
  source 1..29: mol_type = unassigned DNA
gtgtagatctcggtggtcgccgtatcatt

SEQ ID NO: 4 - DNA, 24 bp, unidentified
  source 1..24: mol_type = unassigned DNA
atctcgtatgccgtcttctgcttg

SEQ ID NO: 5 - DNA, 17 bp, unidentified
  source 1..17: mol_type = unassigned DNA
aatgatacggcgaccga

SEQ ID NO: 6 - DNA, 17 bp, synthetic construct
  source 1..17: mol_type = other DNA
tcggtcgccgtatcatt

SEQ ID NO: 7 - protein, 198 aa, unidentified [sequence omitted]

SEQ ID NO: 8 - protein, 188 aa, unidentified
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKENHERTFKAWEGLHENSVRLSRQLRRILLPLYEVDDLRDAFRTLGL

SEQ ID NO: 9 - protein, 198 aa, unidentified
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 10 - protein, 236 aa, unidentified
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 11 - protein, 191 aa, unidentified
MSRKIWRSSGKNTTNHVEVNFIKKFTSERHFHPSISCSITWFLSWSPCWECSQAIREFLSQHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 12 - protein, 229 aa, unidentified
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 13 - protein, 224 aa, unidentified
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 14 - protein, 230 aa, unidentified
MAQKEEAAAATEAAAATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 15 - protein, 224 aa, unidentified
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 16 - protein, 199 aa, unidentified
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 17 - protein, 202 aa, unidentified
MDGSPASRPRHLMDPNTFTFNFNNDLSVRGRHQTYLCYEVERLDNGTWVPMDERRGFLHNKAKNVPCGDYGCHVELRFLCEVPSWQLDPAQTYRVTWFISWSPCFRKGCAGQVRAFLQENKHVRLRIFAARIYDYDPRYQEALRTLRDAGAQVSIMTYEEFKHCWDTFVDRQGRPFQPWDGLDEHSQALSGRLRAILQNQGN

SEQ ID NO: 18 - protein, 199 aa, unidentified
MEASPASGPRHLMDPCVFTSNFNNGIRWHKTYLCYEVERLDNGTWVKMDQHRGFLHNQARNPLYGLDGRHAELRFLGLLPYWQLDPAQIYRVTWFISWSPCFSWGCARQVRAFLQENTHVRLRIFAARIYDYDPLYKEALQMLRDAGAQVSIMTYDEFEYCWNTFVDHQGCPFQPWDGLEEHSQALSGKLQAILLNQGN

SEQ ID NO: 19 - protein, 199 aa, unidentified
MEASPASRPGHLMDPQVFTSNFNNGIRWHKTYLCYEVERLDNGTWVKMDQHRGFLHNQAKNLFCGFYGRHAELCFLDLVPSLQLDPAQTYRVTWFISWSPCFSWGCAEQVRAFLQENTHVRLRLFAARIYDYDPLYKEALQMLRGAGAQVSIMTYHEFKHCWDTFVDHQGRPFQPWDGLEEHSQALSGRLQAILQNQGN

SEQ ID NO: 20 - protein, 199 aa, unidentified
TEASPASGPRHLMDPHIFTSNFNNGIGRRKTYLCYEVERLDNGTSVKMDQHRGFLHNQAKNLLCGFYGRHAELCFLDLVPSLQLDPAQIYRVTWFISWSPCFSWGCAGQVRAFLQENTHVRLRIFAARIYDYDPLYKEALQMLRDAGAQVSIMTYDEFKHCWDTFVDHQGCPFQPWDGLEEHSQALSGRLRAILQNQGN

SEQ ID NO: 21 - protein, 199 aa, unidentified
VEASPASGPRHLMDPHIFTSNFNNVIGRHKTYLCYEVERLDNGTWVKMDQHRGFLHNQAKNLLCGFYGRHAELRFLDLVPSLQLDPAQIYRVTWFISWSPCFSWGCAGQVRAFLQENTHVRLHIFAARIYDYDPLYKEALQMLRDAGAQVSIMTYDEFKHCWDTFVDHQGCPFQPWDGLEEHSQALSGRLRAILQNQGN

SEQ ID NO: 22 - protein, 382 aa, unidentified
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 23 - protein, 382 aa, unidentified
MNPQIRNPMERMYRGTFYNNFENEPILYGRSYNWLCYEVKIKRGRSNLLWNTGVFRGQMYSQPEHHAEMCFLSWFCGNQLPAYKCFQITWFVSWTPCPDCVAKLAEFLAEYPNVTLTISTARLYYYWERDYRRALCRLSQAGARMKIMDYEECAYCWENFVYKEGQQFMPWYKFDENYAFLHHTLKEILRHLMDPDTFTFNFNNDPLVLRRHQTYLCYEVERLDNGTWVLMDRHMGFLCNEAKNLLCGFYGRHAELRFLDLVPSLQLDPAQIYRVTWFISWSPCFSWGCAGQVCEFLQENTHMRLRIFAARIYDYDPLYKKALQMLRDAGAQVSIMTYDEFKHCWDTFVYRQGCPFQPWDGLEEHSQALSGRLQAILQNQGN

SEQ ID NO: 24 - protein, 382 aa, unidentified
MNPQIRNPMERMYRRTFNYNFENEPILYGRSYTWLCYEVKIRKDPSKLPWDTGVFRGQMYSKPEHHAEMCFLSWFCGNQLPAHKRFQITWFVSWTPCPDCVAKVAEFLAEYPNVTLTISAARLYYYWETDYRRALCRLRQAGARVKIMDYEEFAYCWENFVYNEDQSFMPWYKFDDNYAFLHHKLKEILRHLMDPDTFTSNFNNDLSVLGRHQTYLCYEVERLDNGTWVPMDQHWGFLCNQAKNVPRGDYGCHAELCFLDQVSSWQLDPAQTYRVTWFISWSPCFSWGCADQVYAFLQENTHVRLRIFAARIYDYNPLYQEALRTLRDAGAQVSIMTYDEFEYCWDTFVDRQGRPFQPWDGLDEHSQALSGRLRAILQNQGN

SEQ ID NO: 25 - protein, 190 aa, unidentified
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 26 - protein, 190 aa, unidentified
MNPQIRNPMKAMYPGTFYFQFKNLWEANDRNETWLCFTVEGIKRRSVVSWKTGVFRNQVDSETHCHAERCFLSWFCDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 27 - protein, 190 aa, unidentified
MNPQIRNPMKAMDPGTFYFQFKNLWEANNRNETWLCFTVEVIKQHSTVSWETGVFRNQVDLETHCHAERCFLSWFCEDILSPNTDYQVTWYTSWSPCLDCAGEVAKFLARHNNVMLTIYTARLYYSQYPNYQQGLRSLSEKGVSVKIMDYEDFKYCWEKFVYDDGEPFKPWKGLKTSFRFLKRRLREILQ

SEQ ID NO: 28 - protein, 386 aa, unidentified
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 29 - protein, 386 aa, unidentified
MNPQIRNPMERMYRRTFYNHFENEPILYGRSYTWLCYEVKIKRGCSNLIWDTGVFRGPVLPKLQSNHRQEVYFQFENHAEMCFFSWFCGNRLPANRRFQITWFVSWNPCLPCVVKVTKFLAEHPNVTLTISAARLYYYQDREWRRVLRRLHKAGARVKIMDYKDFAHCWENFVYNEGQQFMPWYKFDDNYASLHRTLKEILRNPMEAMYPHVFYFHFKNLLKACGRNESWLCFTVDVTEHHPPVSWKRGVFRNPVDPETHCHAERCFLSWFCDDILSPNTNYQVTWYTSWSPCPECAREVAEFLARHSNVKLTIFTARLYHFWNTDYQEGLCSLSQEGASVKIMSYKDFVSCWKNFVYSDDEPFKPWKGLKTNFRLLKTMLREILQ

SEQ ID NO: 30 - protein, 374 aa, unidentified
MNPQIRNPMERMYRRTFNYNFENEPILYGRSYTWLCYEVKIRKDPSKLPWDTGVFRGQVYFQPQYHAEMCFLSWFCGNQLPAYKRFQITWFVSWNPCPDCVAKVTEFLAEHPNVTLTISVARLYYYRGKDWRRALCRLHQAGARVKIMDYEEFAYCWENFVYNEGQSFMPWDKFDDNYAFLHHKLKEILRNPMKAMYPHTFYFHFENLQKAYGRNETWLCFAVEIIKQHSTVPWKTGVFRNQVDPETHCHAERCFLSWFCDNTLSPKKNYQVTWYISWSPCPECAGEVAEFLATHSNVKLTIYTARLYYFWDTDYQEGLRSLSEEGASMEIMGYEDFKYCWENFVYNDGEPFKPWKGINTNFRFLERRLWKILQ

SEQ ID NO: 31 - protein, 373 aa, unidentified
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 32 - protein, 373 aa, unidentified
MKPQFRNTVERMYRGTFSYNFNNRPILSRRNTVWLCYEVKTKGPSRPPLDAKIFRGQVYFQPQYHAEMCFLSWFCGNQLPAYKCFQITCFVSWTPCPDCVAKLAEFLAEHPNVTLTISAARLYYYWERDYRRALRRLRQAGARVKIMDDEEFAYCWENFVYSEGQPFMPWHKFDDNYAFLHRTLKEILRNPMEAMYPHIFYFHFKNLLKAYGRNESWLCFTMEVIKHHSPVSWKRGVFRNQVDSETHCHAERCFLSWFCDDILSPNTNYQVTWYTSWSPCPECAGEVAEFLARHSNVNLTIFTARLYYFWDTDYQEGLRSLNQEGASVKIMGYKDFKYCWENFVYNDDEPFKPWKGLKYNFLFLDSKLQEILE

SEQ ID NO: 33 - protein, 373 aa, unidentified
MQPQYRNTVERMYRGTFFYNFNNRPILSRRNTVWLCYEVKTRGPSMPTWDTKIFRGQVYSKPEHHAEMCFLSRFCGNQLPAYKRFQITWFVSWTPCPDCVAKVAEFLAEHPNVTLTISAARLYYYWETDYRRALCRLRQAGARVKIMDYEEFAYCWENFVYNEGQSFMPWDKFDDNYAFLHHKLKEILRNPMEATYPHIFYFHFKNLRKAYGRNETWLCFTMEIIKQHSTVSWETGVFRNQVDPESRCHAERCFLSWFCEDILSPNTDYQVTWYTSWSPCLDCAGEVAEFLARHSNVKLAIFAARLYYFWDTHYQQGLRSLSEKGASVEIMGYKDFKYCWENFVYNGDEPFKPWKGLKYNFLFLDSKLQEILE

SEQ ID NO: 34 - protein, 384 aa, unidentified
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 35 - protein, 384 aa, unidentified
MTPQFRNTVERMYRDTFSYNFNNRPILSRRNTVWLCYEVKTKDPSRPPLDAKIFRGQVYSELKYHPEMRFFHWFSKWRKLHRDQEYEVTWYISWSPCTKCTRNVATFLAEDPKVTLTIFVARLYYFWDQDYQEALRSLCQKRDGPRATMKIMNYDEFQHCWSKFVYSQRELFEPWNNLPKYYMLLHIMLGEILRHSMDPPTFTSNFNNEHWVRGRHETYLCYEVERLHNDTWVLLNQRRGFLCNQAPHKHGFLEGRHAELCFLDVIPFWKLDLHQDYRVTCFTSWSPCFSCAQEMAKFISNKKHVSLCIFAARIYDDQGRCQEGLRTLAEAGAKISIMTYSEFKHCWDTFVYHQGCPFQPWDGLEEHSQALSGRLQAILQNQGN

SEQ ID NO: 36 - protein, 384 aa, unidentified
MNPQIRNMVEPMDPRTFVSNFNNRPILSGLNTVWLCCEVKTKDPSGPPLDAKIFQGKVLRSKAKYHPEMRFLQWFREWRQLHHDQEYKVTWYVSWSPCTRCANSVATFLAKDPKVTLTIFVARLYYFWKPNYQQALRILCQKRDGPHATMKIMNYNEFQDCWNKFVDGRGKPFKPWNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRTLHRDGAKIAMMNYSEFEYCWDTFVDCQGCPFQPWDGLDEHSQALSERLRAILQNQGN

SEQ ID NO: 37 - protein, 200 aa, unidentified
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEKPLSFNPYKMLEELDKNSRAIKRRLERIKIPGVRAQGRYMDILCDAEV

SEQ ID NO: 38 - protein, 210 aa, unidentified
MALLTAETFRLQFNNKLRLRRPYYRRKTLLCYQLTPQNGSMPTRGYFKNKKKCHAEICFINEIKSMGLDETQCYQVTCYLTWSPCSSCAWKLVDFIKAHDHLNLRIFASRLYYHWCKRQQEGLRLLCGSQVPVEVMGFPEFADCWENFVDHEKPLSFDPSKMLEELDKNSQAIKRRLERIKSRSVDVLENGLRSLQLGPVTPSSSRSNSR

SEQ ID NO: 39 - protein, 210 aa, unidentified
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 40 - protein, 367 aa, unidentified
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 41 - protein, 368 aa, unidentified
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQSFQMPSGQLQPNLPPDVRAPVVFVLVPLRDLPPMHMGQNPNKPRNIVRHLNMPQMSFQETEDLGRLPTGRSVEIVEITERFASSKEADEKKKKKKGKK

SEQ ID NO: 42 - protein, 374 aa, unidentified
MEPLYEEILTQGGTIVKPYYWLSLSLGCTNCPYHIRTGEEARVPYTEFHQTFGFPWSTYPQTKHLTFYELRSSSKNLIQKGLASNCTGSHNHPEAMLFEKNGYLDAVIFHNSNIRHIILYSNNSPCNEAKHCCISKMYNFLMNYPEVTLSVFFSQLYHTEKQFPTSAWNRKALQSLASLWPQVTLSPICGGLWHAILEKFVSNISGSTVPQPFIAGRILADRYNTYEINSIIAAKPYFTDGLLSRQKENQNREAWAAFEKHPLGSAAPAQRQPTRGQDPRTPAVLMLVSNRDLPPIHVGSTPQKPRTVVRHLNMLQLSSFKVKDVKKPPSGRPVEEVEVMKESARSQKANKKNRSQWKKQTLVIKPRICRLLER

SEQ ID NO: 43 - protein, 2136 aa, unidentified
MSRSRHARPSRLVRKEDVNKKKKNSQLRKTTKGANKNVASVKTLSPGKLKQLIQERDVKKKTEPKPPVPVRSLLTRAGAARMNLDRTEVLFQNPESLTCNGFTMALRSTSLSRRLSQPPLVVAKSKKVPLSKGLEKQHDCDYKILPALGVKHSENDSVPMQDTQVLPDIETLIGVQNPSLLKGKSQETTQFWSQRVEDSKINIPTHSGPAAEILPGPLEGTRCGEGLFSEETLNDTSGSPKMFAQDTVCAPFPQRATPKVTSQGNPSIQLEELGSRVESLKLSDSYLDPIKSEHDCYPTSSLNKVIPDLNLRNCLALGGSTSPTSVIKFLLAGSKQATLGAKPDHQEAFEATANQQEVSDTTSFLGQAFGAIPHQWELPGADPVHGEALGETPDLPEIPGAIPVQGEVFGTILDQQETLGMSGSVVPDLPVFLPVPPNPIATFNAPSKWPEPQSTVSYGLAVQGAIQILPLGSGHTPQSSSNSEKNSLPPVMAISNVENEKQVHISFLPANTQGFPLAPERGLFHASLGIAQLSQAGPSKSDRGSSQVSVTSTVHVVNTTVVTMPVPMVSTSSSSYTTLLPTLEKKKRKRCGVCEPCQQKTNCGECTYCKNRKNSHQICKKRKCEELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTIQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 44 - protein, 2007 aa, unidentified
MSRSRPAKPSKSVKTKLQKKKDIQMKTKTSKQAVRHGASAKAVNPGKPKQLIKRRDGKKETEDKTPTPAPSFLTRAGAARMNRDRNQVLFQNPDSLTCNGFTMALRRTSLSWRLSQRPVVTPKPKKVPPSKKQCTHNIQDEPGVKHSENDSVPSQHATVSPGTENGEQNRCLVEGESQEITQSCPVFEERIEDTQSCISASGNLEAEISWPLEGTHCEELLSHQTSDNECTSPQECAPLPQRSTSEVTSQKNTSNQLADLSSQVESIKLSDPSPNPTGSDHNGFPDSSFRIVPELDLKTCMPLDESVYPTALIRFILAGSQPDVFDTKPQEKTLITTPEQVGSHPNQVLDATSVLGQAFSTLPLQWGFSGANLVQVEALGKGSDSPEDLGAITMLNQQETVAMDMDRNATPDLPIFLPKPPNTVATYSSPLLGPEPHSSTSCGLEVQGATPILTLDSGHTPQLPPNPESSSVPLVIAANGTRAEKQFGTSLFPAVPQGFTVAAENEVQHAPLDLTQGSQAAPSKLEGEISRVSITGSADVKATAMSMPVTQASTSSPPCNSTPPMVERRKRKACGVCEPCQQKANCGECTYCKNRKNSHQICKKRKCEVLKKKPEATSQAQVTKENKRPQREKKPKVLKTDFNNKPVNGPKSESMDCSRRGHGEEEQRLDLITHPLENVRKNAGGMTGIEVEKWAPNKKSHLAEGQVKGSCDANLTGVENPQPSEDDKQQTNPSPTFAQTIRNGMKNVHCLPTDTHLPLNKLNHEEFSKALGNNSSKLLTDPSNCKDAMSVTTSGGECDHLKGPRNTLLFQKPGLNCRSGAEPTIFNNHPNTHSAGSRPHPPEKVPNKEPKDGSPVQPSLLSLMKDRRLTLEQVVAIEALTQLSEAPSESSSPSKPEKDEEAHQKTASLLNSCKAILHSVRKDLQDPNVQGKGLHHDTVVFNGQNRTFKSPDSFATNQALIKSQGYPSSPTAEKKGAAGGRAPFDGFENSHPLPIESHNLENCSQVLSCDQNLSSHDPSCQDAPYSQIEEDVAAQLTQLASTINHINAEVRNAESTPESLVAKNTKQKHSQEKRMVHQKPPSSTQTKPSVPSAKPKKAQKKARATPHANKRKKKPPARSSQENDQKKQEQLAIEYSKMHDIWMSSKFQRFGQSSPRSFPVLLRNIPVFNQILKPVTQSKTPSQHNELFPPINQIKFTRNPELAKEKVKVEPSDSLPTCQFKTESGGQTFAEPADNSQGQPMVSVNQEAHPLPQSPPSNQCANIMAGAAQTQFHLGAQENLVHQIPPPTLPGTSPDTLLPDPASILRKGKVLHFDGITVVTEKREAQTSSNGPLGPTTDSAQSEFKESIMDLLSKPAKNLIAGLKEQEAAPCDCDGGTQKEKGPYYTHLGAGPSVAAVRELMETRFGQKGKAIRIEKIVFTGKEGKSSQGCPVAKWVIRRSGPEEKLICLVRERVDHHCSTAVIVVLILLWEGIPRLMADRLYKELTENLRSYSGHPTDRRCTLNKKRTCTCQGIDPKTCGASFSFGCSWSMYFNGCKFGRSENPRKFRLAPNYPLHEKQLEKNLQELATVLAPLYKQMAPVAYQNQVEYEEVAGDCRLGNEEGRPFSGVTCCMDFCAHSHKDIHNMHNGSTVVCTLIRADGRDTNCPEDEQLHVLPLYRLADTDEFGSVEGMKAKIKSGAIQVNGPTRKRRLRFTEPVPRCGKRAKMKQNHNKSGSHNTKSFSSASSTSHLVKDESTDFCPLQASSAETSTCTYSKTASGGFAETSSILHCTMPSGAHSGANAAAGECTGTVQPAEVAAHPHQSLPTADSPVHAEPLTSPSEQLTSNQSNQQLPLLSNSQKLASCQVEDERHPEADEPQHPEDDNLPQLDEFWSDSEEIYADPSFGGVAIAPIHGSVLIECARKELHATTSLRSPKRGVPFRVSLVFYQHKSLNKPNHGFDINKIKCKCKKVTKKKPADRECPDVSPEANLSHQIPSRVASTLTRDNVVTVSPYSLTHVAGPYNRWV

SEQ ID NO: 45 - protein, 2002 aa, unidentified
MEQDRTNHVEGNRLSPFLIPSPPICQTEPLATKLQNGSPLPERAHPEVNGDTKWHSFKSYYGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGLEEVQNINRRNSPYSQTMKSSACKIQVSCSNNTHLVSENKEQTTHPELFAGNKTQNLHHMQYFPNNVIPKQDLLHRCFQEQEQKSQQASVLQGYKNRNQDMSGQQAAQLAQQRYLIHNHANVFPVPDQGGSHTQTPPQKDTQKHAALRWHLLQKQEQQQTQQPQTESCHSQMHRPIKVEPGCKPHACMHTAPPENKTWKKVTKQENPPASCDNVQQKSIIETMEQHLKQFHAKSLFDHKALTLKSQKQVKVEMSGPVTVLTRQTTAAELDSHTPALEQQTTSSEKTPTKRTAASVLNNFIESPSKLLDTPIKNLLDTPVKTQYDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 46 - protein, 1912 aa, unidentified
MEQDRTTHAEGTRLSPFLIAPPSPISHTEPLAVKLQNGSPLAERPHPEVNGDTKWQSSQSCYGISHMKGSQSSHESPHEDRGYSRCLQNGGIKRTVSEPSLSGLHPNKILKLDQKAKGESNIFEESQERNHGKSSRQPNVSGLSDNGEPVTSTTQESSGADAFPTRNYNGVEIQVLNEQEGEKGRSVTLLKNKIVLMPNGATVSAHSEENTRGELLEKTQCYPDCVSIAVQSTASHVNTPSSQAAIELSHEIPQPSLTSAQINFSQTSSLQLPPEPAAMVTKACDADNASKPAIVPGTCPFQKAEHQQKSALDIGPSRAENKTIQGSMELFAEEYYPSSDRNLQASHGSSEQYSKQKETNGAYFRQSSKFPKDSISPTTVTPPSQSLLAPRLVLQPPLEGKGALNDVALEEHHDYPNRSNRTLLREGKIDHQPKTSSSQSLNPSVHTPNPPLMLPEQHQNDCGSPSPEKSRKMSEYLMYYLPNHGHSGGLQEHSQYLMGHREQEIPKDANGKQTQGSVQAAPGWIELKAPNLHEALHQTKRKDISLHSVLHSQTGPVNQMSSKQSTGNVNMPGGFQRLPYLQKTAQPEQKAQMYQVQVNQGPSPGMGDQHLQFQKALYQECIPRTDPSSEAHPQAPSVPQYHFQQRVNPSSDKHLSQQATETQRLSGFLQHTPQTQASQTPASQNSNFPQICQQQQQQQLQRKNKEQMPQTFSHLQGSNDKQREGSCFGQIKVEESFCVGNQYSKSSNFQTHNNTQGGLEQVQNINKNFPYSKILTPNSSNLQILPSNDTHPACEREQALHPVGSKTSNLQNMQYFPNNVTPNQDVHRCFQEQAQKPQQASSLQGLKDRSQGESPAPPAEAAQQRYLVHNEAKALPVPEQGGSQTQTPPQKDTQKHAALRWLLLQKQEQQQTQQSQPGHNQMLRPIKTEPVSKPSSYRYPLSPPQENMSSRIKQEISSPSRDNGQPKSIIETMEQHLKQFQLKSLCDYKALTLKSQKHVKVPTDIQAAESENHARAAEPQATKSTDCSVLDDVSESDTPGEQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCKKKAEPKKAKTKKAARKRSSLENCSSRTEKGKSSSHTKLMENASHMKQMTAQPQLSGPVIRQPPTLQRHLQQGQRPQQPQPPQPQPQTTPQPQPQPQHIMPGNSQSVGSHCSGSTSVYTRQPTPHSPYPSSAHTSDIYGDTNHVNFYPTSSHASGSYLNPSNYMNPYLGLLNQNNQYAPFPYNGSVPVDNGSPFLGSYSPQAQSRDLHRYPNQDHLTNQNLPPIHTLHQQTFGDSPSKYLSYGNQNMQRDAFTTNSTLKPNVHHLATFSPYPTPKMDSHFMGAASRSPYSHPHTDYKTSEHHLPSHTIYSYTAAASGSSSSHAFHNKENDNIANGLSRVLPGFNHDRTASAQELLYSLTGSSQEKQPEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 47 - protein, 1795 aa, unidentified
MSQFQVPLAVQPDLPGLYDFPQRQVMVGSFPGSGLSMAGSESQLRGGGDGRKKRKRCGTCEPCRRLENCGACTSCTNRRTHQICKLRKCEVLKKKVGLLKEVEIKAGEGAGPWGQGAAVKTGSELSPVDGPVPGQMDSGPVYHGDSRQLSASGVPVNGAREPAGPSLLGTGGPWRVDQKPDWEAAPGPAHTARLEDAHDLVAFSAVAEAVSSYGALSTRLYETFNREMSREAGNNSRGPRPGPEGCSAGSEDLDTLQTALALARHGMKPPNCNCDGPECPDYLEWLEGKIKSVVMEGGEERPRLPGPLPPGEAGLPAPSTRPLLSSEVPQISPQEGLPLSQSALSIAKEKNISLQTAIAIEALTQLSSALPQPSHSTPQASCPLPEALSPPAPFRSPQSYLRAPSWPVVPPEEHSSFAPDSSAFPPATPRTEFPEAWGTDTPPATPRSSWPMPRPSPDPMAELEQLLGSASDYIQSVFKRPEALPTKPKVKVEAPSSSPAPAPSPVLQREAPTPSSEPDTHQKAQTALQQHLHHKRSLFLEQVHDTSFPAPSEPSAPGWWPPPSSPVPRLPDRPPKEKKKKLPTPAGGPVGTEKAAPGIKPSVRKPIQIKKSRPREAQPLFPPVRQIVLEGLRSPASQEVQAHPPAPLPASQGSAVPLPPEPSLALFAPSPSRDSLLPPTQEMRSPSPMTALQPGSTGPLPPADDKLEELIRQFEAEFGDSFGLPGPPSVPIQDPENQQTCLPAPESPFATRSPKQIKIESSGAVTVLSTTCFHSEEGGQEATPTKAENPLTPTLSGFLESPLKYLDTPTKSLLDTPAKRAQAEFPTCDCVEQIVEKDEGPYYTHLGSGPTVASIRELMEERYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLAGDNPKEEEVLRKSFQDLATEVAPLYKRLAPQAYQNQVTNEEIAIDCRLGLKEGRPFAGVTACMDFCAHAHKDQHNLYNGCTVVCTLTKEDNRCVGKIPEDEQLHVLPLYKMANTDEFGSEENQNAKVGSGAIQVLTAFPREVRRLPEPAKSCRQRQLEARKAAAEKKKIQKEKLSTPEKIKQEALELAGITSDPGLSLKGGLSQQGLKPSLKVEPQNHFSSFKYSGNAVVESYSVLGNCRPSDPYSMNSVYSYHSYYAQPSLTSVNGFHSKYALPSFSYYGFPSSNPVFPSQFLGPGAWGHSGSSGSFEKKPDLHALHNSLSPAYGGAEFAELPSQAVPTDAHHPTPHHQQPAYPGPKEYLLPKAPLLHSVSRDPSPFAQSSNCYNRSIKQEPVDPLTQAEPVPRDAGKMGKTPLSEVSQNGGPSHLWGQYSGGPSMSPKRTNGVGGSWGVFSSGESPAIVPDKLSSFGASCLAPSHFTDGQWGLFPGEGQQAASHSGGRLRGKPWSPCKFGNSTSALAGPSLTEKPWALGAGDFNSALKGSPGFQDKLWNPMKGEEGRIPAAGASQLDRAWQSFGLPLGSSEKLFGALKSEEKLWDPFSLEEGPAEEPPSKGAVKEEKGGGGAEEEEEELWSDSEHNFLDENIGGVAVAPAHGSILIECARRELHATTPLKKPNRCHPTRISLVFYQHKNLNQPNHGLALWEAKMKQLAERARARQEEAARLGLGQQEAKLYGKKRKWGGTVVAEPQQKEKKGVVPTRQALAVPTDSAVTVSSYAYTKVTGPYSRWI

SEQ ID NO: 48 - protein, 1803 aa, unidentified
MSQFQVPLAVQPDLSGLYDFPQGQVMVGGFQGPGLPMAGSETQLRGGGDGRKKRKRCGTCDPCRRLENCGSCTSCTNRRTHQICKLRKCEVLKKKAGLLKEVEINAREGTGPWAQGATVKTGSELSPVDGPVPGQMDSGPVYHGDSRQLSTSGAPVNGAREPAGPGLLGAAGPWRVDQKPDWEAASGPTHAARLEDAHDLVAFSAVAEAVSSYGALSTRLYETFNREMSREAGSNGRGPRPESCSEGSEDLDTLQTALALARHGMKPPNCTCDGPECPDFLEWLEGKIKSMAMEGGQGRPRLPGALPPSEAGLPAPSTRPPLLSSEVPQVPPLEGLPLSQSALSIAKEKNISLQTAIAIEALTQLSSALPQPSHSTSQASCPLPEALSPSAPFRSPQSYLRAPSWPVVPPEEHPSFAPDSPAFPPATPRPEFSEAWGTDTPPATPRNSWPVPRPSPDPMAELEQLLGSASDYIQSVFKRPEALPTKPKVKVEAPSSSPAPVPSPISQREAPLLSSEPDTHQKAQTALQQHLHHKRNLFLEQAQDASFPTSTEPQAPGWWAPPGSPAPRPPDKPPKEKKKKPPTPAGGPVGAEKTTPGIKTSVRKPIQIKKSRSRDMQPLFLPVRQIVLEGLKPQASEGQAPLPAQLSVPPPASQGAASQSCATPLTPEPSLALFAPSPSGDSLLPPTQEMRSPSPMVALQSGSTGGPLPPADDKLEELIRQFEAEFGDSFGLPGPPSVPIQEPENQSTCLPAPESPFATRSPKKIKIESSGAVTVLSTTCFHSEEGGQEATPTKAENPLTPTLSGFLESPLKYLDTPTKSLLDTPAKKAQSEFPTCDCVEQIVEKDEGPYYTHLGSGPTVASIRELMEDRYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLTGDNPKEEEVLRNSFQDLATEVAPLYKRLAPQAYQNQVTNEDVAIDCRLGLKEGRPFSGVTACMDFCAHAHKDQHNLYNGCTVVCTLTKEDNRCVGQIPEDEQLHVLPLYKMASTDEFGSEENQNAKVSSGAIQVLTAFPREVRRLPEPAKSCRQRQLEARKAAAEKKKLQKEKLSTPEKIKQEALELAGVTTDPGLSLKGGLSQQSLKPSLKVEPQNHFSSFKYSGNAVVESYSVLGSCRPSDPYSMSSVYSYHSRYAQPGLASVNGFHSKYTLPSFGYYGFPSSNPVFPSQFLGPSAWGHGGSGGSFEKKPDLHALHNSLNPAYGGAEFAELPGQAVATDNHHPIPHHQQPAYPGPKEYLLPKVPQLHPASRDPSPFAQSSSCYNRSIKQEPIDPLTQAESIPRDSAKMSRTPLPEASQNGGPSHLWGQYSGGPSMSPKRTNSVGGNWGVFPPGESPTIVPDKLNSFGASCLTPSHFPESQWGLFTGEGQQSAPHAGARLRGKPWSPCKFGNGTSALTGPSLTEKPWGMGTGDFNPALKGGPGFQDKLWNPVKVEEGRIPTPGANPLDKAWQAFGMPLSSNEKLFGALKSEEKLWDPFSLEEGTAEEPPSKGVVKEEKSGPTVEEDEEELWSDSEHNFLDENIGGVAVAPAHCSILIECARRELHATTPLKKPNRCHPTRISLVFYQHKNLNQPNHGLALWEAKMKQLAERARQRQEEAARLGLGQQEAKLYGKKRKWGGAMVAEPQHKEKKGAIPTRQALAMPTDSAVTVSSYAYTKVTGPYSRWI

SEQ ID NO: 49 - protein, 400 aa, unidentified
MRICIFMARGLEGCGVTKFSLEQRDWFIKNGHEVTLVYAKDKSFTRTSSHDHKSFSIPVILAKEYDKALKLVNDCDILIINSVPATSVQEATINNYKKLLDNIKPSIRVVVYQHDHSVLSLRRNLGLEETVRRADVIFSHSDNGDFNKVLMKEWYPETVSLFDDIEEAPTVYNFQPPMDIVKVRSTYWKDVSEINMNINRWIGRTTTWKGFYQMFDFHEKFLKPAGKSTVMEGLERSPAFIAIKEKGIPYEYYGNREIDKMNLAPNQPAQILDCYINSEMLERMSKSGFGYQLSKLNQKYLQRSLEYTHLELGACGTIPVFWKSTGENLKFRVDNTPLTSHDSGIIWFDENDMESTFERIKELSSDRALYDREREKAYEFLYQHQDSSFCFKEQFDIITK

SEQ ID NO: 50 - protein, 351 aa, unidentified
MKIAIINMGNNVINFKTVPSSETIYLFKVISEMGLNVDIISLKNGVYTKSFDEVDVNDYDRLIVVNSSINFFGGKPNLAILSAQKFMAKYKSKIYYLFTDIRLPFSQSWPNVKNRPWAYLYTEEELLIKSPIKVISQGINLDIAKAAHKKVDNVIEFEYFPIEQYKIHMNDFQLSKPTKKTLDVIYGGSFRSGQRESKMVEFLFDTGLNIEFFGNAREKQFKNPKYPWTKAPVFTGKIPMNMVSEKNSQAIAALIIGDKNYNDNFITLRVWETMASDAVMLIDEEFDTKHRIINDARFYVNNRAELIDRVNELKHSDVLRKEMLSIQHDILNKTRAKKAEWQDAFKKAIDL

SEQ ID NO: 51 - protein, 10 aa, unidentified
HPAVEXPCXC

SEQ ID NO: 52 - protein, 27 aa, unidentified
HXEXSWSTPCXCXFXLXRLIYXLXLXM

SEQ ID NO: 53-54 - skipped (2 entries)

SEQ ID NO: 55 - DNA, 30 bp, unidentified
  source 1..30: mol_type = unassigned DNA
agtgtagatctcggtggtcgccgtatcatt

SEQ ID NO: 56 - DNA, 30 bp, unidentified
  source 1..30: mol_type = unassigned DNA
tgtgtagatctcggtggtcgccgtatcatt

SEQ ID NO: 57 - DNA, 30 bp, unidentified
  source 1..30: mol_type = unassigned DNA
cgtgtagatctcggtggtcgccgtatcatt

SEQ ID NO: 58 - DNA, 30 bp, unidentified
  source 1..30: mol_type = unassigned DNA
ggtgtagatctcggtggtcgccgtatcatt

SEQ ID NO: 59 - DNA, 25 bp, unidentified
  source 1..25: mol_type = unassigned DNA
aatctcgtatgccgtcttctgcttg

SEQ ID NO: 60 - DNA, 25 bp, unidentified
  source 1..25: mol_type = unassigned DNA
tatctcgtatgccgtcttctgcttg

SEQ ID NO: 61 - DNA, 25 bp, unidentified
  source 1..25: mol_type = unassigned DNA
catctcgtatgccgtcttctgcttg

SEQ ID NO: 62 - DNA, 25 bp, unidentified
  source 1..25: mol_type = unassigned DNA
gatctcgtatgccgtcttctgcttg

SEQ ID NO: 63 - DNA, 18 bp, unidentified
  source 1..18: mol_type = unassigned DNA
atcggtcgccgtatcatt

SEQ ID NO: 64 - DNA, 18 bp, unidentified
  source 1..18: mol_type = unassigned DNA
ttcggtcgccgtatcatt

SEQ ID NO: 65 - DNA, 18 bp, unidentified
  source 1..18: mol_type = unassigned DNA
ctcggtcgccgtatcatt

SEQ ID NO: 66 - DNA, 18 bp, unidentified
  source 1..18: mol_type = unassigned DNA
gtcggtcgccgtatcatt

SEQ ID NO: 67-70 - skipped (4 entries)